(19) United States
(12) Patent Application Publication
(10) Pub. No.: US 2012/0128646 A1
(43) Pub. Date: May 24, 2012

(54) METHODS AND COMPOSITIONS FOR THE TREATMENT OF AUTOIMMUNE DISEASE

(76) Inventors: Kathryn Haskins, Denver, CO (US); Thomas Delong, Denver, CO (US); John W. Kappler, Denver, CO (US); Brian Stadinski, N. Easton, MA (US); Nichole Reisdorph, Centennial, CO (US); Rick Reisdorph, Centennial, CO (US)

(21) Appl. No.: 13/147,921

(22) PCT Filed: Feb. 16, 2010
(86) PCT No.: PCT/US10/24300

§ 371 Date: Jan. 23, 2012

Related U.S. Application Data
(60) Provisional application No. 61/153,107, filed on Feb. 17, 2009.

(51) Int. Cl. A61K 38/02; C07K 7/08; A61P 3/10; G01N 33/566; A61K 31/715; A61K 35/14; C07K 2/00; C12Q 1/02

(52) U.S. Cl. 424/93.71; 530/300; 530/327; 435/29; 436/501; 435/7.24; 435/7.1; 514/7.3; 514/54; 435/7.92

(57) ABSTRACT

The present invention is related to the development and treatment of autoimmune disease. Autoimmune diseases can result from tissue damage caused by the activation of autoreactive T cells by autoantigens. For example, peptide fragments of naturally occurring proteins (i.e., for example, chromogranin A) may activate autoreactive T cells that result in the destruction of pancreatic β islet cells, possibly by the release of inflammatory cytokines (i.e., for example, interferon-γ). One naturally occurring biologically active chromogranin A peptide fragment, WE14, may comprise a diabetogenic autoantigen. Truncation and extension analysis of WE14 indicates that the stimulating binding register of WE14 occupies only half of the mouse IA^{g7} peptide binding groove, leaving positions p1 to p4 empty. Inhibition of autoantigen-autoreactive T cell binding may provide therapeutic as well a prophylactic treatments for autoimmune diseases

### STATEMENT OF GOVERNMENTAL SUPPORT

This invention was made with government support awarded by the National Institutes of Health (grant numbers DK50561, and T32 AI007405, BioResources Core of Diabetes & Endocrinology Research Center (grant numbers P30 DK057516, 5 U19-AI050864, AI17134, AI18785), the National Center for Research Resources (grant number S10RR023703). The government has certain rights in the invention.

### FIELD OF INVENTION

The present invention is related to the development and treatment of autoimmune disease. Autoimmune diseases can result from tissue damage caused by the activation of autoreactive T cells by autoantigens. For example, peptide fragments of naturally occurring proteins (i.e., for example, chromogranin A) may activate autoreactive T cells that result in the destruction of pancreatic β islet cells. Inhibition of autoantigen-autoreactive T cell binding may provide therapeutic as well as prophylactic treatments for autoimmune diseases.

### BACKGROUND

Human autoimmune diseases have a striking genetic association with particular alleles of major histocompatability complex (“MHC”) class I or class II genes. The field was established by the seminal discovery of HLA-B27 linked susceptibility to ankylosing spondylitis, a chronic inflammatory joint disease (Brewerton (et al., 1973; Schlosstein et al., 1973). MHC associated susceptibility has now been documented for a variety of human autoimmune diseases, including type 1 diabetes mellitus (T1D), rheumatoid arthritis (RA), pemphigus vulgaris (PV), multiple sclerosis (MS) and myasthenia gravis (MG), just to name a few (Todd et al., 1987; Ahmed et al., 1990; Ahmed et al. 1991; Lanchbury & Panayi, 1991; Spielman & Nathenson, 1982; Protti et al., 1993).

While associations between MHC alleles and disease states have implicated autoimmunity in the etiology of these diseases, a large body of clinical and epidemiological evidence suggests that infections may be important in the induction of autoimmunity. For example, particular viral infections frequently precede autoimmune myocarditis and type I diabetes (IDDM) (Rose et al., 1986; Ray et al., 1980). Environmental agents also influence the risk of developing multiple sclerosis as demonstrated by migration studies. Individuals that migrate after age 15 carry the risk for developing MS associated with their geographic origin while individuals who migrate earlier in life acquire the risk of the geographical region to which they migrated (Kurtzke, 1985). These studies are consistent with the hypothesis that a group of pathogens that are relatively ubiquitous in a certain geographic region influence the risk of developing multiple sclerosis (MS). The mechanism(s) leading to clonal expansion of MBP reactive T cells remain to be identified but could involve recognition of viral peptides with sufficient structural similarity to the immunodominant MBP peptide. The initiation of autoimmunity by such a mechanism could then lead to sensitization to other CNS self antigens by determinant spreading (Lehmann et al., 1992; Kaufman et al. 1993; Tisch et al., 1993). Consonant with this hypothesis, it has been noted that inflammatory CNS disease can follow infection with a number of common viral pathogens, such as measles and rubella. On the other hand, the absence of virus in the CNS of these patients and reactivity to myelin basic protein in these patients suggest an autoimmune mechanism (Johnson et al., 1984).

Efforts to identify sequence homologies between self peptide epitopes that might be involved in autoimmunity and various bacterial and viral pathogens have therefore been made. These homology searches have focused on alignments with sequence identity. No success has been reported using such alignments in identifying epitopes from pathogens that could cross react with presumably pathogenic T cell lines from human patients with autoimmune disease (Oldstone, 1990). A sequence identity was recently found between an epitope in a Coxsackie virus protein and GAD65, suspected of being an autoantigen in diabetes. These peptides could reciprocally generate polyclonal T cell lines from mice that cross react with the other peptides (Tian, et al., 1994). No evidence, however, was provided that these peptides could stimulate clones from diabetic mice (or humans).

Recent developments in the field, in particular the identification of allele specific peptide binding motifs have transformed the field (Madden et al., 1991; Rotschke & Falk, 1991). Based on this knowledge the structural basis for MHC linked susceptibility to autoimmune diseases can be reassessed at a level of detail sufficient for solving longstanding questions in the field. Motifs for peptide binding to several MHC class I and class II molecules have been defined by sequence analysis of naturally processed peptides and by mutational analysis of known epitopes. MHC class I bound peptides were found to be short (generally 8-10 amino acids long) and to possess two dominant MHC anchor residues; MHC class II bound peptides were found to be longer and more heterogeneous in size (Madden et al., 1991; Rotschke & Falk, 1991; Jardetzky et al. 1991, Chicz et al. 1993). Due to the size heterogeneity, however, it has proven more difficult to define MHC class II binding motifs based on sequence alignments. More recently, a crystal structure for HLA-DR1 demonstrated that there is a dominant hydrophobic anchor residue close to the N-terminus of the peptide and that secondary anchor residues are found at several other peptide positions (Brown et al., 1993). Even this work, however, could not provide a detailed description of the binding pockets of HLA-DR proteins, the particular residues involved in the formation of these pockets of the structural requirements or antigens for MHC binding.

What is needed is a method to identify specific autoantigens responsible for the development of autoimmune disease in order to provide therapeutics as well as prophylactic regimens designed to reduce and/or prevent the progression of these diseases.

### SUMMARY OF THE INVENTION

The present invention is related to the development and treatment of autoimmune disease. Autoimmune diseases can result from tissue damage caused by the activation of autoreactive T cells by autoantigens. For example, fragments of naturally occurring proteins (i.e., for example, chromogranin A) may activate autoreactive T cells that result in the destruction of pancreatic β islet cells. Inhibition of autoantigen-autoreactive T cell binding may provide therapeutic as well a prophylactic treatments for autoimmune diseases.

In one embodiment, the present invention contemplates an isolated amino acid sequence, wherein the sequence comprises at least a portion of chromogranin A or a chromogranin A-like peptide. In one embodiment, the amino acid sequence comprises a portion of the chromogranin A protein. In one embodiment, the amino acid sequence comprises chromogranin A-like activity. In one embodiment, the chromogranin A-like activity comprises autoreactive T cell activation. In one embodiment, the amino acid sequence comprises a human amino acid sequence of WSKMDQLAKELTAE (SEQ ID NO: 1). In one embodiment, the amino acid sequence comprises a modified human amino acid sequence selected from the group consisting of REWEDKRWSKMDQLAKELTA (SEQ ID NO: 2), EDKRWSKMDQLAKELTAE (SEQ ID NO: 3), EDKRWSKMDQLA (SEQ ID NO: 4), WEDKRWSKMDQLAKELTAE (SEQ ID NO: 5), WEDKRWSKMDQLAKELT (SEQ ID NO: 6), WEDKRWSKMDQLAKEL (SEQ ID NO: 7), WEDKRWSKMDQLAKE (SEQ ID NO: 8), WEDKRWSKMDQLAK (SEQ ID NO: 9), or WEDKRWSKMDQLA (SEQ ID NO: 10). In one embodiment, the amino acid sequence comprises a mouse amino acid sequence of WSRMDQLAKELTAE (SEQ ID NO: 11). In one embodiment, the amino acid sequence comprises a modified mouse amino acid sequence selected from the group consisting of REWEDKRWS RMDQLAKELTA (SEQ ID NO: 12), EDKRWSRMDQLAKELTAE (SEQ ID NO: 13), EDKRWSRMDQLA (SEQ ID NO: 14), WEDKRWS RMDQLAKELTAE (SEQ ID NO: 15), WEDKRWSRMDQLAKELT (SEQ ID NO: 16), WEDKRWSRMDQLAKEL (SEQ ID NO: 17), WED KRWSRMDQLAKE (SEQ ID NO: 18), WEDKRWSRMDQLAK (SEQ ID NO: 19), or WEDKRWSRMDQLA (SEQ ID NO: 20). In one embodiment, the amino acid sequence comprises a synthetic peptide mimotope. In one embodiment, the mimotope is selected from the group comprising SRLGLWVRME (SEQ ID NO: 21), SRLVLWVRME (SEQ ID NO: 22), SRLTLWVRME (SEQ ID NO: 23), SRLSLWVRME (SEQ ID NO: 24), SRLALWVRME (SEQ ID NO: 25), SRLPLWVRME (SEQ ID NO: 26), SRLCLWVRME (SEQ ID NO: 27), SRLYLWVRME (SEQ ID NO: 28), SRLRLWVRME (SEQ ID NO: 29), SRLMLWVRME (SEQ ID NO: 30), SRLHLWVRME (SEQ ID NO: 31), or SRFGLWVRME (SEQ ID NO: 32). In one embodiment, the mimotope comprises an amino acid sequence selected from the group consisting of HRPIWARMD (SEQ ID NO: 33), HLAIWAKMD (SEQ ID NO: 34), HLAIWARMD (SEQ ID NO: 35), or HIPIWARMD (SEQ ID NO: 36). In one embodiment, the chromogranin A portion comprises a peptide mimotope selected from the group comprising RLGLWVRME (SEQ ID NO: 37), RVGQWARME (SEQ ID NO: 38), RLGGWARMM (SEQ ID NO: 39), ELMEWWKMM (SEQ ID NO: 40), or PRITWTRMG (SEQ ID NO: 41). In one embodiment, the peptide comprises at least one post-translational enzymatic modification. In one embodiment, the peptide comprises between approximately nine and forty nine amino acids. In one embodiment, the post-translational enzymatic modifications selected from the group comprising hydrolysis, acylation, phosphorylation, ubiquitination, sumoylation, deamidation, citrullination, disulfide bridges, proteolytic cleavage, and/or multimerization. In one embodiment, the post-translational modification is located at an amino acid residue selected from the group consisting of T, A, M, or Q. In one embodiment, the peptide is purified. In one embodiment, the chromogranin A is a human chromogranin A. In one embodiment, the peptide comprises a chimeric peptide.

In one embodiment, the present invention contemplates a method, comprising: a) providing; i) a biological sample derived from a human patient comprising at least one risk marker for type 1 diabetes, wherein the sample is suspected of comprising an amino acid sequence comprising at least a portion of a chromogranin A-like peptide; ii) a test composition comprising isolated T cells; b) contacting said T cells with said sample under conditions that activate the T-cells; and c) detecting the T-cell activation, thereby diagnosing said type 1 diabetes. In one embodiment, the risk marker comprises an autoantibody profile. In one embodiment, the risk marker comprises an major histocompatability complex molecule associated with type 1 diabetes. In one embodiment, the risk marker comprises detecting urinary glucose. In one embodiment, the risk marker comprises elevated blood glucose. In one embodiment, the isolated T cells comprise human T cells. In one embodiment, the activation is detected by measuring at least one other inflammatory cytokine. In one embodiment, the inflammatory cytokine comprises interferon-γ. In one embodiment, the activation is detected by measuring a change in at least one T cell surface molecule. In one embodiment, the surface marker comprises CD69. In one embodiment, the surface receptor comprises a susceptible MHC molecule. In one embodiment, the peptide is between fourteen and forty amino acids. In one embodiment, the amino acid sequence comprises a human amino acid sequence of WSKMDQLAKELTAE (SEQ ID NO: 1). In one embodiment, the amino acid sequence comprises a modified human amino acid sequence selected from the group consisting of REWEDKRWSKMDQLAKELTA (SEQ ID NO: 2), EDKRWSKMDQLAKELTAE (SEQ ID NO: 3), EDKRWSKMDQLA (SEQ ID NO: 4), WEDKRWSKMDQLAKELTAE (SEQ ID NO: 5), WEDKRWSKMDQLAKELT (SEQ ID NO: 6), WEDKRWSKMDQLAKEL (SEQ ID NO: 7), WEDKRWSKMDQLAKE (SEQ ID NO: 8), WEDKRWSKMDQLAK (SEQ ID NO: 9), or WEDKRWSKMDQLA (SEQ ID NO: 10). In one embodiment, the amino acid sequence comprises a synthetic peptide mimotope. In one embodiment, the mimotope is selected from the group comprising SRLGLWVRME (SEQ ID NO: 21), SRLVLWVRME (SEQ ID NO: 22), SRLTLWVRME (SEQ ID NO: 23), SRLSLWVRME (SEQ ID NO: 24), SRLALWVRME (SEQ ID NO: 25), SRLPLWVRME (SEQ ID NO: 26), SRLCLWVRME (SEQ ID NO: 27), SRLYLWVRME (SEQ ID NO: 28), SRLRLWVRME (SEQ ID NO: 29), SRLMLWVRME (SEQ ID NO: 30), SRLHLWVRME (SEQ ID NO: 31), or SRFGLWVRME (SEQ ID NO: 32). In one embodiment, the mimotope comprises an amino acid sequence selected from the group consisting of HRPIWARMD (SEQ ID NO: 33), HLAIWAKMD (SEQ ID NO: 34), HLAIWARMD (SEQ ID NO: 35), or HIPIWARMD (SEQ ID NO: 36). In one embodiment, the chromogranin A portion comprises a peptide mimotope selected from the group comprising RLGLWVRME (SEQ ID NO: 37), RVGQWARME (SEQ ID NO: 38), RLGGWARMM (SEQ ID NO: 39), ELMEWWKMM (SEQ ID NO: 40), or PRITWTRMG (SEQ ID NO: 41). In one embodiment, the peptide comprises at least one post-translational enzymatic modification. In one embodiment, the peptide comprises between approximately nine and forty nine amino acids. In one embodiment, the post-translational enzymatic modifications selected from the group comprising hydrolysis, acylation, phosphorylation, ubiquitination, sumoylation, deamidation, citrullination, disulfide bridges, proteolytic cleavage, and/or multimerization. In one embodiment, the post-translational modification is located at an amino acid residue selected from the group consisting of T, A, M, or Q. In one embodiment, the sample is a blood sample. In one embodiment, the blood sample is selected from the group comprising a whole blood sample, a plasma sample, or a serum sample. In one embodiment, the sample comprises a tissue sample. In one embodiment, the tissue sample comprises a pancreatic tissue sample. In one embodiment, the pancreatic tissue sample comprises islet cells. In one embodiment, diabetes is diagnosed when measuring an interferon production of at least 50 ng/ml. In one embodiment, diabetes is diagnosed when measuring an interferon production of at least 40 ng/ml. In one embodiment, diabetes is diagnosed when measuring an interferon production of at least 30 ng/ml. In one embodiment, diabetes is diagnosed wherein measuring an interferon production of at least 20 ng/ml. In one embodiment, diabetes is diagnosed when measuring an interferon production of at least 10 ng/ml. In one embodiment, diabetes is diagnosed when measuring an upregulation of at least one other inflammatory cytokine. In one embodiment, diabetes is diagnosed when measuring upregulation of at least one surface receptor.

In one embodiment, the present invention contemplates a method, comprising: a) providing; i) a biological sample derived from a mammal comprising at least one risk marker for type 1 diabetes, wherein the sample is suspected of comprising an amino acid comprising at least a portion of a chromogranin A-like peptide; ii) a test panel comprising at least two diabetogenic CD4+ Th1 T cell clones; b) mixing individually said sample with said first clone and the second clone under conditions that activate the T cell clone; and c) detecting the T cell clone activation, thereby diagnosing said type 1 diabetes. In one embodiment, the risk marker comprises an autoantibody panel. In one embodiment, the risk marker comprises an major histocompatability complex molecule associated with type 1 diabetes. In one embodiment, the risk marker comprises detecting urinary glucose. In one embodiment, the risk marker comprises elevated blood glucose. In one embodiment, the activation is detected by measuring interferon-γ. In one embodiment, the activation is detected by measuring at least one cytokine. In one embodiment, the activation is detected by measuring at least one T cell surface receptor. In one embodiment, the surface receptor comprises CD69. In one embodiment, the activation is detected by measuring T cell proliferation. In one embodiment, the T cell clone activation is measured by techniques including but not limited to, ELISA, ELISPOT, or flow cytometry. In one embodiment, the mammal comprises a non-human mammal selected from the group consisting of a mouse, a rat, or a rabbit. In one embodiment, the peptide is between fourteen and forty amino acids. In one embodiment, the amino acid sequence comprises a mouse amino acid sequence of WSRMDQLAKELTAE (SEQ ID NO: 11). In one embodiment, the amino acid sequence comprises a modified mouse amino acid sequence selected from the group consisting of REWEDKRWS RMDQLAKELTA (SEQ ID NO: 12), EDKRWSRMDQLAKELTAE (SEQ ID NO: 13), EDKRWSRMDQLA (SEQ ID NO: 14), WEDKRWS RMDQLAKELTAE (SEQ ID NO: 15), WEDKRWSRMDQLAKELT (SEQ ID NO: 16), WEDKRWSRMDQLAKEL (SEQ ID NO: 17), WED KRWSRMDQLAKE (SEQ ID NO: 18), WEDKRWSRMDQLAK (SEQ ID NO: 19), or WEDKRWSRMDQLA (SEQ ID NO: 20). In one embodiment, the amino acid sequence comprises a synthetic peptide mimotope. In one embodiment, the mimotope is selected from the group comprising SRLGLWVRME (SEQ ID NO: 21), SRLVLWVRME (SEQ ID NO: 22), SRLTLWVRME (SEQ ID NO: 23), SRLSLWVRME (SEQ ID NO: 24), SRLALWVRME (SEQ ID NO: 25), SRLPLWVRME (SEQ ID NO: 26), SRLCLWVRME (SEQ ID NO: 27), SRLYLWVRME (SEQ ID NO: 28), SRLRLWVRME (SEQ ID NO: 29), SRLMLWVRME (SEQ ID NO: 30), SRLHLWVRME (SEQ ID NO: 31), or SRFGLWVRME (SEQ ID NO: 32). In one embodiment, the mimotope comprises an amino acid sequence selected from the group consisting of HRPIWARMD (SEQ ID NO: 33), HLAIWAKMD (SEQ ID NO: 34), HLAIWARMD (SEQ ID NO: 35), or HIPIWARMD (SEQ ID NO: 36). In one embodiment, the chromogranin A portion comprises a peptide mimotope selected from the group comprising RLGLWVRME (SEQ ID NO: 37), RVGQWARME (SEQ ID NO: 38), RLGGWARMM (SEQ ID NO: 39), ELMEWWKMM (SEQ ID NO: 40), or PRITWTRMG (SEQ ID NO: 41). In one embodiment, the peptide comprises at least one post-translational enzymatic modification. In one embodiment, the peptide comprises between approximately nine and forty nine amino acids. In one embodiment, the post-translational enzymatic modifications selected from the group comprising hydrolysis, acylation, phosphorylation, ubiquitination, sumoylation, deamidation, citrullination, disulfide bridges, proteolytic cleavage, and/or multimerization. In one embodiment, the post-translational modification is located at an amino acid residue selected from the group consisting of T, A, M, or Q. In one embodiment, the diabetogenic T cell clones may be selected from the group comprising BDC-2.5, BDC-10.1, BDC-5.10.3, or PD-12.4.4. In one embodiment, the sample is a blood sample. In one embodiment, the blood sample is selected from the group comprising a whole blood sample, a plasma sample, or a serum sample. In one embodiment, the sample comprises a tissue sample. In one embodiment, the tissue sample comprises a pancreatic tissue sample. In one embodiment, the pancreatic tissue sample comprises islet cells. In one embodiment, diabetes is diagnosed when measuring an interferon production of at least 50 ng/ml. In one embodiment, diabetes is diagnosed when measuring an interferon production of at least 40 ng/ml. In one embodiment, diabetes is diagnosed when measuring an interferon production of at least 30 ng/ml. In one embodiment, diabetes is diagnosed wherein measuring an interferon production of at least 20 ng/ml. In one embodiment, diabetes is diagnosed when measuring an interferon production of at least 10 ng/ml. In one embodiment, diabetes is diagnosed when measuring an upregulation of at least one cytokine. In one embodiment, diabetes is diagnosed when measuring upregulation of at least one surface receptor.

In one embodiment, the present invention contemplates a method, comprising: a) providing; i) a biological sample derived from a patient exhibiting at least one risk marker of having type 1 diabetes, wherein said sample is suspected of comprising at least one diabetogenic biomarker; ii) a peptide comprising specific affinity for the biomarker; b) mixing said peptide with said sample under conditions such that said biomarker binds to said peptide, thereby forming a peptide-biomarker complex; and c) detecting said peptide-biomarker complex, thereby diagnosing said type 1 diabetes. In one embodiment, the risk marker comprises an autoantibody profile. In one embodiment, the risk marker comprises an major histocompatability complex associated with type 1 diabetes. In one embodiment, the risk marker comprises detecting urinary glucose. In one embodiment, the risk marker comprises elevated blood glucose. In one embodiment, the diabetogenic biomarker comprises an amino acid sequence. In one embodiment, the amino acid sequence comprises at least a portion of a chromogranin A-like peptide. In one embodiment, the amino acid sequence comprises a peptide derived from a beta pancreatic cell membrane. In one embodiment, the amino acid sequence comprises a peptide derived from a beta pancreatic cell cytosol. In one embodiment, the amino acid sequence comprises a peptide derived from a beta pancreatic cell nucleus. In one embodiment, the amino acid sequence comprises an autoantibody. In one embodiment, the diabetogenic biomarker comprises a nucleic acid sequence. In one embodiment, the nucleic acid sequence comprises a deoxyribonucleic acid sequence. In one embodiment, the nucleic acid sequence comprises a ribonucleic acid sequence. In one embodiment, the ribonucleic acid sequence comprises a messenger ribonucleic acid sequence. In one embodiment, the ribonucleic acid sequence comprises a mitochondrial ribonucleic acid sequence. In one embodiment, the nucleic acid encodes at least a portion of a chromogranin A-like peptide. In one embodiment, the nucleic acid sequence encodes a peptide derived from a beta pancreatic cell membrane. In one embodiment, the nucleic acid sequence encodes a peptide derived from a beta pancreatic cell cytosol. In one embodiment, the nucleic acid sequence encodes a peptide derived from a beta pancreatic cell nucleus. In one embodiment, the biomarker comprises a nucleic acid sequence encoding the autoantibody. In one embodiment, the biomarker comprises an autoreactive T cell. In one embodiment, the biomarker comprises an beta islet cell membrane. In one embodiment, the diabetogenic biomarker comprises a cell receptor. In one embodiment, the cell receptor comprises an IA^{g7} receptor. In one embodiment, the cell receptor comprises a CD69 receptor. In one embodiment, the biomarker comprises a polysaccharide. In one embodiment, the polysaccharide comprises a glucopolysaccaride. In one embodiment, the cell receptor comprises a lipid. In one embodiment, the lipid comprises a phospholipid. In one embodiment, the peptide is between fourteen and forty amino acids. In one embodiment, the patient comprises a human. In one embodiment, the patient comprises a non-human mammal selected from the group consisting of a mouse, a rat, or a rabbit. In one embodiment, the amino acid sequence comprises a human amino acid sequence of WSKMDQLAKELTAE (SEQ ID NO: 1). In one embodiment, the amino acid sequence comprises a modified human amino acid sequence selected from the group consisting of REWEDKRWSKMDQLAKELTA (SEQ ID NO: 2), EDKRWSKMDQLAKELTAE (SEQ ID NO: 3), EDKRWSKMDQLA (SEQ ID NO: 4), WEDKRWSKMDQLAKELTAE (SEQ ID NO: 5), WEDKRWSKMDQLAKELT (SEQ ID NO: 6), WEDKRWSKMDQLAKEL (SEQ ID NO: 7), WEDKRWSKMDQLAKE (SEQ ID NO: 8), WEDKRWSKMDQLAK (SEQ ID NO: 9), or WEDKRWSKMDQLA (SEQ ID NO: 10). In one embodiment, the amino acid sequence comprises a mouse amino acid sequence of WSRMDQLAKELTAE (SEQ ID NO: 11). In one embodiment, the amino acid sequence comprises a modified mouse amino acid sequence selected from the group consisting of REWEDKRWS RMDQLAKELTA (SEQ ID NO: 12), EDKRWSRMDQLAKELTAE (SEQ ID NO: 13), EDKRWSRMDQLA (SEQ ID NO: 14), WEDKRWS RMDQLAKELTAE (SEQ ID NO: 15), WEDKRWSRMDQLAKELT (SEQ ID NO: 16), WEDKRWSRMDQLAKEL (SEQ ID NO: 17), WED KRWSRMDQLAKE (SEQ ID NO: 18), WEDKRWSRMDQLAK (SEQ ID NO: 19), or WEDKRWSRMDQLA (SEQ ID NO: 20). In one embodiment, the amino acid sequence comprises a synthetic peptide mimotope. In one embodiment, the mimotope is selected from the group comprising SRLGLWVRME (SEQ ID NO: 21), SRLVLWVRME (SEQ ID NO: 22), SRLTLWVRME (SEQ ID NO: 23), SRLSLWVRME (SEQ ID NO: 24), SRLALWVRME (SEQ ID NO: 25), SRLPLWVRME (SEQ ID NO: 26), SRLCLWVRME (SEQ ID NO: 27), SRLYLWVRME (SEQ ID NO: 28), SRLRLWVRME (SEQ ID NO: 29), SRLMLWVRME (SEQ ID NO: 30), SRLHLWVRME (SEQ ID NO: 31), or SRFGLWVRME (SEQ ID NO: 32). In one embodiment, the mimotope comprises an amino acid sequence selected from the group consisting of HRPIWARMD (SEQ ID NO: 33), HLAIWAKMD (SEQ ID NO: 34), HLAIWARMD (SEQ ID NO: 35), or HIPIWARMD (SEQ ID NO: 36). In one embodiment, the chromogranin A portion comprises a peptide mimotope selected from the group comprising RLGLWVRME (SEQ ID NO: 37), RVGQWARME (SEQ ID NO: 38), RLGGWARMM (SEQ ID NO: 39), ELMEWWKMM (SEQ ID NO: 40), or PRITWTRMG (SEQ ID NO: 41). In one embodiment, the peptide comprises at least one post-translational enzymatic modification. In one embodiment, the peptide comprises between approximately nine and forty nine amino acids. In one embodiment, the post-translational enzymatic modifications selected from the group comprising hydrolysis, acylation, phosphorylation, ubiquitination, sumoylation, deamidation, citrullination, disulfide bridges, proteolytic cleavage, and/or multimerization. In one embodiment, the post-translational modification is located at an amino acid residue selected from the group consisting of T, A, M, or Q. In one embodiment, the peptide further comprises a label. In one embodiment, the label comprises a detectable label. In one embodiment, the label comprises an affinity label. In one embodiment, the label comprises a fluorescent label. In one embodiment, the label comprises a radioactive label. In one embodiment, the sample is a blood sample. In one embodiment, the blood sample is selected from the group comprising a whole blood sample, a plasma sample, or a serum sample. In one embodiment, the sample comprises a tissue sample. In one embodiment, the tissue sample comprises a pancreatic tissue sample. In one embodiment, the pancreatic tissue sample comprises islet cells.

In one embodiment, the present invention contemplates a method, comprising: a) providing; i) a biological sample derived from a patient exhibiting at least one risk marker of having type 1 diabetes, wherein said sample is suspected of comprising at least one diabetogenic biomarker; ii) a diagnostic antibody comprising specific affinity for the at least one biomarker; b) mixing said diagnostic antibody with said sample under conditions such that said biomarker binds to said diagnostic antibody, thereby forming a diagnostic antibody-biomarker complex; and c) detecting said diagnostic antibody-biomarker complex, thereby diagnosing said type 1 diabetes. In one embodiment, the risk marker comprises an autoantibody profile. In one embodiment, the risk marker comprises a major histocompatability complex associated with type 1 diabetes. In one embodiment, the risk marker comprises detecting urinary glucose. In one embodiment, the risk marker comprises elevated blood glucose. In one embodiment, the diabetogenic biomarker comprises an amino acid sequence. In one embodiment, the amino acid sequence comprises at least a portion of a chromogranin A-like peptide. In one embodiment, the amino acid sequence comprises a peptide derived from a beta pancreatic cell membrane. In one embodiment, the amino acid sequence comprises a peptide derived from a beta pancreatic cell cytosol. In one embodiment, the amino acid sequence comprises a peptide derived from a beta pancreatic cell nucleus. In one embodiment, the amino acid sequence comprises an autoantibody. In one embodiment, the diabetogenic biomarker comprises a nucleic acid sequence. In one embodiment, the nucleic acid sequence comprises a deoxyribonucleic acid sequence. In one embodiment, the nucleic acid sequence comprises a ribonucleic acid sequence. In one embodiment, the ribonucleic acid sequence comprises a messenger ribonucleic acid sequence. In one embodiment, the ribonucleic acid sequence comprises a mitochondrial ribonucleic acid sequence. In one embodiment, the nucleic acid encodes at least a portion of a chromogranin A-like peptide. In one embodiment, the nucleic acid sequence encodes a peptide derived from a beta pancreatic cell membrane. In one embodiment, the nucleic acid sequence encodes a peptide derived from a beta pancreatic cell cytosol. In one embodiment, the nucleic acid sequence encodes a peptide derived from a beta pancreatic cell nucleus. In one embodiment, the biomarker comprises a nucleic acid sequence encoding the autoantibody. In one embodiment, the biomarker comprises an autoreactive T cell. In one embodiment, the biomarker comprises a beta islet cell membrane. In one embodiment, the diabetogenic biomarker comprises a cell receptor. In one embodiment, the cell receptor comprises an IA^{g7} receptor. In one embodiment, the cell receptor comprises a CD69 receptor. In one embodiment, the biomarker comprises a polysaccharide. In one embodiment, the polysaccharide comprises a glucopolysaccaride. In one embodiment, the cell receptor comprises a lipid. In one embodiment, the lipid comprises a phospholipid. In one embodiment, the diagnostic antibody comprises a detectable label. In one embodiment, the label comprises an affinity label. In one embodiment, the label comprises a fluorescent label. In one embodiment, the label comprises a radioactive label. In one the detecting comprises an enzyme linked immunosorbant assay. In one embodiment, the detecting comprising an immunofluorescent sandwich assay. In one embodiment, the peptide is between fourteen and forty amino acids. In one embodiment, the patient comprises a human. In one embodiment, the patient comprises a non-human mammal selected from the group consisting of a mouse, a rat, or a rabbit. In one embodiment, the amino acid sequence comprises a human amino acid sequence of WSKMDQLAKELTAE (SEQ ID NO: 1). In one embodiment, the amino acid sequence comprises a modified human amino acid sequence selected from the group consisting of REWEDKRWSKMDQLAKELTA (SEQ ID NO: 2), EDKRWSKMDQLAKELTAE (SEQ ID NO: 3), EDKRWSKMDQLA (SEQ ID NO: 4), WEDKRWSKMDQLAKELTAE (SEQ ID NO: 5), WEDKRWSKMDQLAKELT (SEQ ID NO: 6), WEDKRWSKMDQLAKEL (SEQ ID NO: 7), WEDKRWSKMDQLAKE (SEQ ID NO: 8), WEDKRWSKMDQLAK (SEQ ID NO: 9), or WEDKRWSKMDQLA (SEQ ID NO: 10). In one embodiment, the amino acid sequence comprises a mouse amino acid sequence of WSRMDQLAKELTAE (SEQ ID NO: 11). In one embodiment, the amino acid sequence comprises a modified mouse amino acid sequence selected from the group consisting of REWEDKRWS RMDQLAKELTA (SEQ ID NO: 12), EDKRWSRMDQLAKELTAE (SEQ ID NO: 13), EDKRWSRMDQLA (SEQ ID NO: 14), WEDKRWS RMDQLAKELTAE (SEQ ID NO: 15), WEDKRWSRMDQLAKELT (SEQ ID NO: 16), WEDKRWSRMDQLAKEL (SEQ ID NO: 17), WED KRWSRMDQLAKE (SEQ ID NO: 18), WEDKRWSRMDQLAK (SEQ ID NO: 19), or WEDKRWSRMDQLA (SEQ ID NO: 20). In one embodiment, the amino acid sequence comprises a synthetic peptide mimotope. In one embodiment, the mimotope is selected from the group comprising SRLGLWVRME (SEQ ID NO: 21), SRLVLWVRME (SEQ ID NO: 22), SRLTLWVRME (SEQ ID NO: 23), SRLSLWVRME (SEQ ID NO: 24), SRLALWVRME (SEQ ID NO: 25), SRLPLWVRME (SEQ ID NO: 26), SRLCLWVRME (SEQ ID NO: 27), SRLYLWVRME (SEQ ID NO: 28), SRLRLWVRME (SEQ ID NO: 29), SRLMLWVRME (SEQ ID NO: 30), SRLHLWVRME (SEQ ID NO: 31), or SRFGLWVRME (SEQ ID NO: 32). In one embodiment, the mimotope comprises an amino acid sequence selected from the group consisting of HRPIWARMD (SEQ ID NO: 33), HLAIWAKMD (SEQ ID NO: 34), HLAIWARMD (SEQ ID NO: 35), or HIPIWARMD (SEQ ID NO: 36). In one embodiment, the chromogranin A portion comprises a peptide mimotope selected from the group comprising RLGLWVRME (SEQ ID NO: 37), RVGQWARME (SEQ ID NO: 38), RLGGWARMM (SEQ ID NO: 39), ELMEWWKMM (SEQ ID NO: 40), or PRITWTRMG (SEQ ID NO: 41). In one embodiment, the peptide comprises at least one post-translational enzymatic modification. In one embodiment, the peptide comprises between approximately nine and forty nine amino acids. In one embodiment, the post-translational enzymatic modifications selected from the group comprising hydrolysis, acylation, phosphorylation, ubiquitination, sumoylation, deamidation, citrullination, disulfide bridges, proteolytic cleavage, and/or multimerization. In one embodiment, the post-translational modification is located at an amino acid residue selected from the group consisting of T, A, M, or Q. In one embodiment, the label comprises a detectable label. In one embodiment, the label comprises an affinity label. In one embodiment, the sample is a blood sample. In one embodiment, the blood sample is selected from the group comprising a whole blood sample, a plasma sample, or a serum sample. In one embodiment, the sample comprises a tissue sample. In one embodiment, the tissue sample comprises a pancreatic tissue sample. In one embodiment, the pancreatic tissue sample comprises islet cells.

In one embodiment, the present invention contemplates a method, comprising: a) providing; i) a patient exhibiting at least one symptom of type 1 diabetes; ii) a pharmaceutical composition comprising a therapeutic agent capable of reducing the at least one symptom of type 1 diabetes; b) administering said composition to said patient under conditions such that said at least one symptom is reduced. In one embodiment, the method further comprises step (c) wherein the administering induces T cell tolerance. In one embodiment, the method further comprises step (c) wherein the administering inhibits an autoantibody associated with diabetes. In one embodiment, the method further comprises step (c) wherein the administering inhibits a pancreatic beta cell surface receptor, wherein the receptor has specific affinity for the autoantibody. In one embodiment, the therapeutic agent comprises an amino acid sequence. In one embodiment, the amino acid sequence comprises at least a portion of a chromogranin A-like peptide. In one embodiment, the amino acid sequence comprises a peptide derived from a beta pancreatic cell membrane. In one embodiment, the amino acid sequence comprises a peptide derived from a beta pancreatic cell cytosol. In one embodiment, the amino acid sequence comprises a peptide derived from a beta pancreatic cell nucleus. In one embodiment, the amino acid sequence comprises an antibody having specific affinity for at least a portion of a chromogranin A-like peptide. In one embodiment, the amino acid sequence comprises an antibody having specific affinity for an autoantibody associated with diabetes. In one embodiment, the antibody comprises a polyclonal antibody. In one embodiment, the antibody comprises a monoclonal antibody. In one embodiment, the therapeutic agent comprises a nucleic acid sequence. In one embodiment, the nucleic acid sequence comprises a deoxyribonucleic acid sequence. In one embodiment, the nucleic acid sequence comprises a ribonucleic acid sequence. In one embodiment, the ribonucleic acid sequence comprises a messenger ribonucleic acid sequence. In one embodiment, the ribonucleic acid sequence comprises a mitochondrial ribonucleic acid sequence. In one embodiment, the nucleic acid sequence comprises an antisense nucleic acid sequence. In one embodiment, the antisense nucleic acid sequence comprises a small interfering ribonucleic acid sequence. In one embodiment, the antisense nucleic acid sequence comprises a silencing ribonucleic acid sequence. In one embodiment, the nucleic acid encodes at least a portion of a chromogranin A-like peptide. In one embodiment, the nucleic acid sequence encodes a peptide derived from a beta pancreatic cell membrane. In one embodiment, the nucleic acid sequence encodes a peptide derived from a beta pancreatic cell cytosol. In one embodiment, the nucleic acid sequence encodes a peptide derived from a beta pancreatic cell nucleus. In one embodiment, the nucleic acid sequence encodes an antibody having specific affinity for the autoantibody associated with diabetes. In one embodiment, the therapeutic agent comprises a small organic molecule. In one embodiment, the small organic molecule has specific affinity for an autoantibody associated with diabetes. In one embodiment, the small organic molecule has specific affinity for an autoreactive T cell surface receptor. In one embodiment, the cell surface receptor comprises an IA^{g7} receptor. In one embodiment, the cell surface receptor comprises a CD69 receptor. In one embodiment, the small organic molecule has specific affinity for a pancreatic beta islet cell surface receptor. In one embodiment, the composition further comprises a molecular or cellular complex. In one embodiment, the patient comprises a human. In one embodiment, the patient comprises a non-human mammal selected from the group including, but not limited to, a mouse, a rat, or a rabbit. In one embodiment, the peptide is linked to a T cell. In one embodiment, the peptide is between fourteen and forty amino acids. In one embodiment, the amino acid sequence comprises a human amino acid sequence of WSKMDQLAKELTAE (SEQ ID NO: 1). In one embodiment, the amino acid sequence comprises a modified human amino acid sequence selected from the group consisting of REWEDKRWSKMDQLAKELTA (SEQ ID NO: 2), EDKRWSKMDQLAKELTAE (SEQ ID NO: 3), EDKRWSKMDQLA (SEQ ID NO: 4), WEDKRWSKMDQLAKELTAE (SEQ ID NO: 5), WEDKRWSKMDQLAKELT (SEQ ID NO: 6), WEDKRWSKMDQLAKEL (SEQ ID NO: 7), WEDKRWSKMDQLAKE (SEQ ID NO: 8), WEDKRWSKMDQLAK (SEQ ID NO: 9), or WEDKRWSKMDQLA (SEQ ID NO: 10). In one embodiment, the amino acid sequence comprises a mouse amino acid sequence of WSRMDQLAKELTAE (SEQ ID NO: 11). In one embodiment, the amino acid sequence comprises a modified mouse amino acid sequence selected from the group consisting of REWEDKRWS RMDQLAKELTA (SEQ ID NO: 12), EDKRWSRMDQLAKELTAE (SEQ ID NO: 13), EDKRWSRMDQLA (SEQ ID NO: 14), WEDKRWS RMDQLAKELTAE (SEQ ID NO: 15), WEDKRWSRMDQLAKELT (SEQ ID NO: 16), WEDKRWSRMDQLAKEL (SEQ ID NO: 17), WED KRWSRMDQLAKE (SEQ ID NO: 18), WEDKRWSRMDQLAK (SEQ ID NO: 19), or WEDKRWSRMDQLA (SEQ ID NO: 20). In one embodiment, the amino acid sequence comprises a synthetic peptide mimotope. In one embodiment, the mimotope is selected from the group comprising SRLGLWVRME (SEQ ID NO: 21), SRLVLWVRME (SEQ ID NO: 22), SRLTLWVRME (SEQ ID NO: 23), SRLSLWVRME (SEQ ID NO: 24), SRLALWVRME (SEQ ID NO: 25), SRLPLWVRME (SEQ ID NO: 26), SRLCLWVRME (SEQ ID NO: 27), SRLYLWVRME (SEQ ID NO: 28), SRLRLWVRME (SEQ ID NO: 29), SRLMLWVRME (SEQ ID NO: 30), SRLHLWVRME (SEQ ID NO: 31), or SRFGLWVRME (SEQ ID NO: 32). In one embodiment, the mimotope comprises an amino acid sequence selected from the group consisting of HRPIWARMD (SEQ ID NO: 33), HLAIWAKMD (SEQ ID NO: 34), HLAIWARMD (SEQ ID NO: 35), or HIPIWARMD (SEQ ID NO: 36). In one embodiment, the chromogranin A portion comprises a peptide mimotope selected from the group comprising RLGLWVRME (SEQ ID NO: 37), RVGQWARME (SEQ ID NO: 38), RLGGWARMM (SEQ ID NO: 39), ELMEWWKMM (SEQ ID NO: 40), or PRITWTRMG (SEQ ID NO: 41). In one embodiment, the peptide comprises a post-translational enzymatic modifications selected from the group comprising hydrolysis, acylation, phosphorylation, ubiquitination, sumoylation, deamidation, citrullination, disulfide bridges, proteolytic cleavage, and/or multimerization. In one embodiment, the post-translational modification is located at an amino acid residue selected from the group consisting of T, A, M, or Q. In one embodiment, the administering is parenteral. In one embodiment, the administering is oral. In one embodiment, the pharmaceutical composition comprises a liposome population. In one embodiment, the pharmaceutical composition is selected from the group consisting of a tablet, a capsule, a controlled release delivery system, or a sachet. In one embodiment, the pharmaceutical composition comprises a liquid.

In one embodiment, the present invention contemplates a kit comprising: a) a first container comprising at least two CD4+ Th1 T cell clones; b) a plurality of containers comprising buffers and reagents capable of detecting T cell activation; and c) a set of instructional materials describing how to detect the T cell activation after contact with a biological sample.

In one embodiment, the present invention contemplates a kit comprising: a) a first container comprising a composition comprising a peptide or antibody having specific affinity for a diabetogenic biomarker; b) a plurality of containers comprising buffers and reagents capable of detecting T cell activation; and c) a set of instructional materials describing how to detect the T cell activation after contacting the composition with a biological sample. In one embodiment, the biological sample comprises said diabetogenic biomarker. In one embodiment, the diabetogenic biomarker is selected from the group comprising an amino acid sequence, a nucleic acid sequence, a polysaccharide, a lipid, or an autoreactive T cell. In one embodiment, the peptide or antibody comprises a detectable label.

In one embodiment, the present invention contemplates a kit comprising: a) a first container comprising a labeled amino acid comprising at least a portion of a chromogranin A-like peptide; b) a plurality of containers comprising buffers and reagents capable of contacting the peptide with a biological sample suspected of comprising diabetogenic autoantibodies; and c) a set of instructional material to detect the autoantibodies and provide a diabetes diagnosis.

In one embodiment, the present invention contemplates a kit comprising: a) a first container comprising a pharmaceutically acceptable composition comprising an amino acid comprising at least a portion of a chromogranin A-like peptide having specific affinity for a diabetogenic autoantigen; b) a plurality of containers comprising buffers and reagent capable of configuring the composition for administration to a patient; and c) a set of instructional material to administer the composition to the patient to reduce diabetes symptoms.

In one embodiment, the present invention contemplates a vector comprising a polynucleotide wherein the polynucleotide encodes an amino acid sequence selected from the group consisting of WSKMDQLAKELTAE (SEQ ID NO: 1), REWEDKRWSKMDQLAKELTA (SEQ ID NO: 2), EDKRWSKMDQLAKELTAE (SEQ ID NO: 3), EDKRWSKMDQLA (SEQ ID NO: 4), WEDKRWSKMDQLAKELTAE (SEQ ID NO: 5), WEDKRWSKMDQLAKELT (SEQ ID NO: 6), WEDKRWSKMDQLAKEL (SEQ ID NO: 7), WEDKRWSKMDQLAKE (SEQ ID NO: 8), WEDKRWSKMDQLAK (SEQ ID NO: 9), WEDKRWSKMDQLA (SEQ ID NO: 10), WSRMDQLAKELTAE (SEQ ID NO: 11), REWEDKRWS RMDQLAKELTA (SEQ ID NO: 12), EDKRWSRMDQLAKELTAE (SEQ ID NO: 13), EDKRWSRMDQLA (SEQ ID NO: 14), WEDKRWS RMDQLAKELTAE (SEQ ID NO: 15), WEDKRWSRMDQLAKELT (SEQ ID NO: 16), WEDKRWSRMDQLAKEL (SEQ ID NO: 17), WED KRWSRMDQLAKE (SEQ ID NO: 18), WEDKRWSRMDQLAK (SEQ ID NO: 19), WEDKRWSRMDQLA (SEQ ID NO: 20), SRLGLWVRME (SEQ ID NO: 21), SRLVLWVRME (SEQ ID NO: 22), SRLTLWVRME (SEQ ID NO: 23), SRLSLWVRME (SEQ ID NO: 24), SRLALWVRME (SEQ ID NO: 25), SRLPLWVRME (SEQ ID NO: 26), SRLCLWVRME (SEQ ID NO: 27), SRLYLWVRME (SEQ ID NO: 28), SRLRLWVRME (SEQ ID NO: 29), SRLMLWVRME (SEQ ID NO: 30), SRLHLWVRME (SEQ ID NO: 31), SRFGLWVRME (SEQ ID NO: 32), HRPIWARMD (SEQ ID NO: 33), HLAIWAKMD (SEQ ID NO: 34), HLAIWARMD (SEQ ID NO: 35), HIPIWARMD (SEQ ID NO: 36), RLGLWVRME (SEQ ID NO: 37), RVGQWARME (SEQ ID NO: 38), RLGGWARMM (SEQ ID NO: 39), ELMEWWKMM (SEQ ID NO: 40), or PRITWTRMG (SEQ ID NO: 41). In one embodiment, the vector is operably linked to a promoter. In one embodiment, the vector is incorporated into an expression platform. In one embodiment, the expression platform comprises a mammalian cell culture. In one embodiment, the expression platform comprises a bacterial cell culture.

### DEFINITIONS

The term “autoreactive T cell activation” as used herein, refers to any means by which a T cell is contacted by an autoantigen thereby stimulating the production of inflammatory cytokines, e.g., IFN-γ. For example, a T cell may be contacted by an amino acid sequence comprising at least a portion of a chromogranin A-like peptide wherein the T cell produces at least one inflammatory cytokine. Alternatively, T cell activation may facilitate interaction with a B cell, wherein autoantibodies associated with an autoimmune disease (i.e., for example, diabetes) are produced.

The term “diabetogenic biomarker” as used herein refers to any compound that is capable of identifying the presence, development, and/or progression of diabetes. For example, such biomarkers may include but are not limited to, amino acid sequences comprising at least a portion of a chromogranin A-like peptide or autoantibodies having specific affinity for an amino acid sequence comprising at least a portion of a chromogranin A-like peptide. Alternatively, such biomarkers may include, but are not limited to, nucleic acid sequences encoding amino acid sequences comprising at least a portion of a chromogranin A-like peptide or autoantibodies having specific affinity for an amino acid sequence comprising at least a portion of a chromogranin A-like peptide. Other biomarkers may be derived from any pancreatic cell location including but not limited to the plasma membrane, cytosol, nucleus, or mitochondria.

The term “autoantibody associated with diabetes” as used herein, refers to any antibody that is generated during the development of diabetes.

The term “at risk for” or “suspected of having” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “risk marker” as used herein, refers to any quantitative and/or qualitative clinical evaluation that can be interpreted by a medical practitioner to suggest a patient may be susceptible to developing a specific disease and/or medical condition. For example, risk markers for diabetes may include, but are not limited to, an autoantibody profile and/or panel, a major histocompatability complex (MHC) molecule associated with disease susceptibility, detectable urinary glucose, or elevated blood glucose.

The term “autoantibody profile” or “autoantibody panel” as used herein, refers to the detection of autoantibodies including, but not limited to, antibodies to pancreatic beta cell autoantigens such as insulin and chromogranin A, antinuclear antibodies (ANA), Ro (SSA) autoantibodies, anticardiolipin antibodies (ACA), systemic lupus erythematosus (SLE) autoantibodies, or thyroid autoantibodies.

The term “a major histocompatability complex associated with type 1 diabetes” as used herein, refers to the identification of any MHC family cell surface antigen complex that regularly appears in the presence of diabetes. Brims et al., “Predominant occupation of the class I MHC molecule H-2 Kwm7 with a single self-peptide suggests a mechanism for its diabetes-protective effect” Int Immunol. (Jan. 21, 2010, Epub). Techniques for measuring MHC have been widely reported and are referenced herein. MHC class 1 molecules may be found on every nucleated cell of the body and are believed to display fragments of proteins from within the cell to T cells. MHC Class II are believed to be heterodimer molecules found on specialized cell types including, but not limited to, macrophages, dendritic cells and B cells, all of which are professional antigen-presenting cells (APCs). The peptides presented by class II molecules are derived from extracellular proteins, hence, the MHC class II-dependent pathway of antigen presentation is called the endocytic or exogenous pathway. MHC Class III molecules encodes for immune components including, but not limited to, complement components (i.e., for example, C2, C4, factor B), cytokines (i.e., for example, TNF-α) and also hsp.

The term “glucose clearance” as used herein, refers to any method by which body tissues extract glucose from the blood. When glucose clearance is decreased, blood glucose levels remain elevated (i.e., for example, a symptom of insulin resistance). Conversely, when glucose clearance is increased, blood glucose levels are lowered towards normal levels. Consequently, one symptom of diabetes is the detection of urinary glucose because a decreased blood glucose clearance results in a prolonged elevation in blood glucose levels, thereby causing renal overflow of glucose into the urine. As a result, a compound may increase glucose clearance (i.e., for example, a proteinase inhibitor) and return blood/urine glucose levels to normal levels, thereby reducing diabetic symptoms.

The term “chromogranin A-like peptide” as used herein, refers to any amino acid sequence comprising a portion of which is either substantially homologous and/or has chromogranin A-like activity as compared to a wild type chromogranin A protein. Chromogranin A or parathyroid secretory protein 1 (gene name CHGA) is a member of the chromogranin/secretogranin (granins) family of neuroendocrine secretory proteins, i.e. it is located in secretory vesicles of neurons and endocrine cells. In humans, chromogranin A protein is encoded by the CHGA gene.

The term “chromogranin A-like activity” as used herein, refers to any amino acid sequence comprising activity that is physiologically comparable to a wild type chromogranin A protein. For example, chromogranin A is the precursor to several functional peptides including vasostatin, pancreastatin, catestatin and parastatin. Consequently, some chromogranin A-like activity comprises a negative modulation of neuroendocrine function for autocrine or paracrine cells. Alternatively, other chromogranin A-like activity may include activation of autoreactive T cells.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “immunoprecipitation” as used herein, refers to any precipitation of a complex of an antibody and its specific antigen. Usually, such a complex may be initiated by the addition of a protein that binds immunoglobulin including, but not limited to, Protein A on an agarose solid support.

The terms “reduce,” “inhibit,” “diminish,” “suppress,” “decrease,” “prevent” and grammatical equivalents (including “lower,” “smaller,” etc.) when in reference to the expression of any symptom (e.g., a withdrawal symptom) in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “inhibitory compound” as used herein, refers to any compound capable of interacting with (i.e., for example, attaching, binding etc) to a binding partner (i.e., for example, a diabetogenic autoantigen) under conditions such that the binding partner becomes unresponsive to its natural ligands. Inhibitory compounds may include, but are not limited to, small organic molecules, antibodies, and proteins/peptides.

The term “attached” or “attaching” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “medium” as used herein, refers to any material, or combination of materials, which serve as a carrier or vehicle for delivering of a therapeutic compound to a biological target. For all practical purposes, therefore, the term “medium” is considered synonymous with the term “carrier”. It should be recognized by those having skill in the art that a medium comprises a carrier, wherein said carrier is attached to a therapeutic compound and said medium facilitates delivery of said carrier to a biological target. Further, a carrier may comprise an attached therapeutic compound wherein said carrier facilitates delivery of said therapeutic compound to a biological target. Preferably, a medium is selected from the group including, but not limited to, foams, gels (including, but not limited to, hydrogels), xerogels, microparticles (i.e., microspheres, liposomes, microcapsules etc.), bioadhesives, or liquids. Specifically contemplated by the present invention is a medium comprising combinations of microparticles with hydrogels, bioadhesives, foams or liquids. Preferably, hydrogels, bioadhesives and foams comprise any one, or a combination of, polymers contemplated herein. Any medium contemplated by this invention may comprise a controlled release formulation. For example, in some cases a medium constitutes a drug delivery system that provides a controlled and sustained release of therapeutic agents over a period of time lasting approximately from 1 day to 6 months.

The term “drug” or “therapeutic agent” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering” a therapeutic compound, as used herein, refers to any method of providing a therapeutic compound to a patient such that the therapeutic compound has its intended effect on the patient. For example, one method of administering is by an indirect mechanism using a medical device such as, but not limited to a catheter, applicator gun, syringe etc. A second exemplary method of administering is by a direct mechanism such as, local tissue administration (i.e., for example, extravascular placement), oral ingestion, transdermal patch, topical, inhalation, suppository etc.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a compound or sequence. In one respect, a compound or sequence may be derived from an organism or particular species. In another respect, a compound or sequence may be derived from a larger complex or sequence.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that some trace impurities may remain.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

The term “biocompatible”, as used herein, refers to any material does not elicit a substantial detrimental response in the host. There is always concern, when a foreign object is introduced into a living body, that the object will induce an immune reaction, such as an inflammatory response that will have negative effects on the host. In the context of this invention, biocompatiblity is evaluated according to the application for which it was designed: for example; a bandage is regarded a biocompatible with the skin, whereas an implanted medical device is regarded as biocompatible with the internal tissues of the body. Preferably, biocompatible materials include, but are not limited to, biodegradable and biostable materials.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “modified human amino acid sequence” as used herein refers to any structural and/or conformational change to a wild type human amino acid sequence. Such changes may including but not limited to, an extension of at least one amino acid residue, a deletion of at least one amino acid residue, or at least one post-translational modification.

The term “modified mouse amino acid sequence” as used herein refers to any structural and/or conformational change to a wild type mouse amino acid sequence. Such changes may including but not limited to, an extension of at least one amino acid residue, a deletion of at least one amino acid residue, or at least one post-translational modification.

The term “peptide mimotope” as used herein refers to any amino acid sequence that comprises substantially similar homology and/or biological activity as a wild type amino acid sequence. Similar homology may be determined by amino acid sequence identity and/or physico-chemical similarity. Similar biological activity may be determined by similarity in secondary, tertiary, and/or quaternary structure between the wild type sequence and the peptide mimotope.

As used herein the term “fragment” when in reference to a protein (as in “a fragment of a given protein”) refers to amino acid sequences that are shorter than the complete protein. For example, a fragment may range in size from four amino acid residues to the complete amino acid sequence minus one amino acid.

The term “portion” when used in reference to a nucleotide sequence refers to nucleic acid sequence that are shorter than the complete nucleotide sequence. A portion may range in size from 5 nucleotide residues to the complete nucleotide sequence minus one nucleic acid residue.

The term “antibody” may refer to an immunoglobulin evoked in animals by an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

The terms “specific binding” or “specifically binding” when used in reference to the interaction of an antibody and a protein or peptide means that the interaction is dependent upon the presence of a particular structure (i.e., for example, an antigenic determinant or epitope) on a protein; in other words an antibody is recognizing and binding to a specific protein structure rather than to proteins in general. For example, if an antibody is specific for epitope “A”, the presence of a protein containing epitope A (or free, unlabelled A) in a reaction containing labeled “A” and the antibody will reduce the amount of labeled A bound to the antibody.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

As used herein, the term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

As used herein, the terms “siRNA” refers to either small interfering RNA, short interfering RNA, or silencing RNA. Generally, siRNA comprises a class of double-stranded RNA molecules, approximately 20-25 nucleotides in length. Most notably, siRNA is involved in RNA interference (RNAi) pathways and/or RNAi-related pathways. wherein the compounds interfere with gene expression.

As used herein, the term “shRNA” refers to any small hairpin RNA or short hairpin RNA. Although it is not necessary to understand the mechanism of an invention, it is believed that any sequence of RNA that makes a tight hairpin turn can be used to silence gene expression via RNA interference. Typically, shRNA uses a vector stably introduced into a cell genome and is constitutively expressed by a compatible promoter. The shRNA hairpin structure may also cleaved into siRNA, which may then become bound to the RNA-induced silencing complex (RISC). This complex binds to and cleaves mRNAs which match the siRNA that is bound to it.

As used herein, the term “microRNA”, “miRNA”, or “μRNA” refers to any single-stranded RNA molecules of approximately 21-23 nucleotides in length, which regulate gene expression. miRNAs may be encoded by genes from whose DNA they are transcribed but miRNAs are not translated into protein (i.e. they are non-coding RNAs). Each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The term “functionally equivalent codon”, as used herein, refers to different codons that encode the same amino acid. This phenomenon is often referred to as “degeneracy” of the genetic code. For example, six different codons encode the amino acid arginine.

A “variant” of a protein is defined as an amino acid sequence which differs by one or more amino acids from a polypeptide sequence or any homolog of the polypeptide sequence. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions (i.e., additions), or both. Guidance in determining which and how many amino acid residues may be substituted, inserted or deleted without abolishing biological or immunological activity may be found using computer programs including, but not limited to, DNAStar® software.

A “variant” of a nucleotide is defined as a novel nucleotide sequence which differs from a reference oligonucleotide by having deletions, insertions and substitutions. These may be detected using a variety of methods (e.g., sequencing, hybridization assays etc.). Included within this definition are alterations to the genomic DNA sequences, the inability of a selected fragments to hybridize under high stringency conditions to a sample of genomic DNA (e.g., using allele-specific oligonucleotide probes), and improper or unexpected hybridization, such as hybridization to a locus other than a normal chromosomal locus for a specific gene (e.g., using fluorescent in situ hybridization (FISH)).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues, respectively, as compared to, for example, the naturally occurring gene or protein.

A “substitution” results from the replacement of one or more nucleotides or amino acids by different nucleotides or amino acids, respectively.

The term “derivative” as used herein, refers to any chemical modification of a nucleic acid or an amino acid. Illustrative of such modifications would be replacement of hydrogen by an alkyl, acyl, or amino group. For example, a nucleic acid derivative would encode a polypeptide which retains essential biological characteristics.

The term “biologically active” refers to any molecule having structural, regulatory or biochemical functions.

The term “immunologically active” defines the capability of a natural, recombinant or synthetic peptide, or any oligopeptide thereof, to induce a specific immune response in appropriate animals or cells and/or to bind with specific antibodies.

The term “antigenic determinant” as used herein refers to that portion of a molecule that is recognized by a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The terms “immunogen,” “antigen,” “immunogenic” and “antigenic” refer to any substance capable of generating antibodies when introduced into an animal. By definition, an immunogen must contain at least one epitope (the specific biochemical unit capable of causing an immune response), and generally contains many more. Proteins are most frequently used as immunogens, but lipid and nucleic acid moieties complexed with proteins may also act as immunogens. The latter complexes are often useful when smaller molecules with few epitopes do not stimulate a satisfactory immune response by themselves.

The term “autoantigen” as used herein, refers to any substance capable of generating autoantibodies or activating autoreactive T cells when introduced to an animal.

The term “antibody” refers to immunoglobulin evoked in animals by an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

Low stringency conditions comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent {50×Denhardt's contains per 500 ml: 5 g Ficoll (Type 400, Pharmacia), 5 g BSA (Fraction V; Sigma)} and 100 μg/ml denatured salmon sperm DNA followed by washing in a solution comprising 5×SSPE, 0.1% SDS at 42° C. when a probe of about 500 nucleotides in length. is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when nucleic acid fragments are employed in hybridization reactions under stringent conditions the hybridization of fragments which contain unique sequences (i.e., regions which are either non-homologous to or which contain less than about 50% homology or complementarity with the fragments are favored. Alternatively, when conditions of “weak” or “low” stringency are used hybridization may occur with nucleic acids that are derived from organisms that are genetically diverse (i.e., for example, the frequency of complementary sequences is usually low between such organisms).

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of a target sequence of interest. In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out using polymerase chain reaction. Dieffenbach C. W. and G. S. Dveksler (1995) In: PCR Primer, a Laboratory Manual, Cold Spring Harbor Press, Plainview, N.Y.

As used herein, the term “polymerase chain reaction” (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers; to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double-stranded DNA at or near a specific nucleotide sequence.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription. Maniatis, T. et al., Science 236:1237 (1987). Promoter and enhancer elements have been isolated from a variety of eukaryotic sources including genes in plant, yeast, insect and mammalian cells and viruses (analogous control elements, i.e., promoters, are also found in prokaryotes). The selection of a particular promoter and enhancer depends on what cell type is to be used to express the protein of interest.

The presence of “splicing signals” on an expression vector often results in higher levels of expression of the recombinant transcript. Splicing signals mediate the removal of introns from the primary RNA transcript and consist of a splice donor and acceptor site. Sambrook, J. et al., In: Molecular Cloning: A Laboratory Manual, 2nd ed., Cold Spring Harbor laboratory Press, New York (1989) pp. 16.7-16.8. A commonly used splice donor and acceptor site is the splice junction from the 16S RNA of SV40.

The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene. Efficient expression of recombinant DNA sequences in eukaryotic cells involves expression of signals directing the efficient termination and polyadenylation of the resulting transcript. Transcription termination signals are generally found downstream of the polyadenylation signal and are a few hundred nucleotides in length.

The term “transfection” or “transfected” refers to the introduction of foreign DNA into a cell.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “Southern blot” refers to the analysis of DNA on agarose or acrylamide gels to fractionate the DNA according to size, followed by transfer and immobilization of the DNA from the gel to a solid support, such as nitrocellulose or a nylon membrane. The immobilized DNA is then probed with a labeled oligodeoxyribonucleotide probe or DNA probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists. J. Sambrook et al. (1989) In: Molecular Cloning: A Laboratory Manual, Cold Spring Harbor Press, NY, pp 9.31-9.58.

The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled oligodeoxyribonucleotide probe or DNA probe to detect RNA species complementary to the probe used. Northern blots are a standard tool of molecular biologists. J. Sambrook, J. et al. (1989) supra, pp 7.39-7.52.

The term “reverse Northern blot” as used herein refers to the analysis of DNA by electrophoresis of DNA on agarose gels to fractionate the DNA on the basis of size followed by transfer of the fractionated DNA from the gel to a solid support, such as nitrocellulose or a nylon membrane. The immobilized DNA is then probed with a labeled oligoribonucleotide probe or RNA probe to detect DNA species complementary to the ribo probe used.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

The term “binding” as used herein, refers to any interaction between an infection control composition and a surface. Such as surface is defined as a “binding surface”. Binding may be reversible or irreversible. Such binding may be, but is not limited to, non-covalent binding, covalent bonding, ionic bonding, Van de Waal forces or friction, and the like. An infection control composition is bound to a surface if it is impregnated, incorporated, coated, in suspension with, in solution with, mixed with, etc.

The term “hybridoma” as used herein, refers to any hybrid cell produced by the fusion of an antibody-producing lymphocyte with a tumor cell and used to culture continuously a specific monoclonal antibody.

The term “post-translational enzymatic modification” as used herein, refers to any chemical changes made to a newly synthesized protein that is mediated by an enzyme. Such new protein synthesis may occur either in vivo or in vitro. The invention contemplates the in vitro “post-translation enzymatic modification” of synthetically made proteins. For example, an in vitro protein synthesis may comprise combinatorial chemistry or cell culture protein expression systems, wherein a post-translational enzymatic modification is made to the newly synthesized protein. Some post-translational enzymatic modifications include, but are not limited to, hydrolysis, acylation, phosphorylation, ubiquitination, sumoylation, deamidation, citrullination, disulfide bridges, proteolytic cleavage, and/or multimerization. These post-translational modifications may be made at any amino acid residue, but preferably at amino acid residues T, A, M, or Q.

### BRIEF DESCRIPTION OF THE FIGURES

FIG. 1 presents exemplary data showing responsivity of a BDC panel comprising four T cell clones to β-membrane autoantigens and NOD APCs. In each culture well, ˜20,000 responder T cells (R) were combined with elicited peritoneal cells (PEC) as APC and 10 μg β-membrane as antigen (Ag). Controls were responder T cells and APC without Ag. Culture SN fractions were harvested after 24 hr and assayed for presence of IFNγ by ELISA.

FIG. 2 presents an illustration of a 30 gauge strainer needle designed to prepare β-cell membrane fractions from whole cell pancreatic tissues.

FIG. 3 presents one embodiment of an improved experimental design for antigen purification and identification.

FIG. 4 presents exemplary data showing separation of β-membrane lysates by size exclusion chromatography (SEC).

FIG. 4A: A comparison of protein fractionation by SEC of membrane lysates from antigenic fresh RIPTag tumor cells (black line) or the non-antigenic NIT-1 cell line (red line).

FIG. 4B: A silver stain of SDS-PAGE gel lanes from the RIPTag SEC-fraction and the corresponding non-antigenic NIT-1 fraction. The bands (e.g., like the one indicated by the red arrow) that appear in the RIPTag fraction but not in the NIT-1 fraction could be candidate antigens for the T-cell clone BDC 2.5.

FIG. 5 presents exemplary data showing an analysis of fractions from size exclusion chromatography/ion exchange chromatography (SEC/IEX) for antigenicity and protein content. IEX was performed on antigenic fractions obtained from the SEC column. A linear salt gradient was applied and monitored by a conductivity meter (dashed red line). The lower part of the figure shows a silver-stained SDS gel of the antigenic fractions—each lane contained about 40-50 individual protein bands. βM=whole beta cell membrane lysate.

FIG. 6 presents one embodiment of the purification of antigen for the T cell clone BDC 2.5 from NOD RIPTAg adenomas.

FIG. 6A: Representative chromatograms from SEC chromatography of 13.8 mg β-membrane lysate.

FIG. 6B: Representative chromatograms from IEX chromatography. Anion exchange chromatography (IEX) of pooled antigenic SEC fractions 60-62. The protein content for each chromatographic fractionation was monitored by its absorption at 280 nm (blue lines). The fractions obtained were tested for the presence of antigen with the T cell clone BDC 2.5 (red lines). One antigen unit (A.U.) causes the production of 100 ng/ml IFN-g under standard antigen assay conditions.

FIG. 6C. Silver-stained, Tricine-Tris Gel Electrophoresis analysis of antigenic fractions from SEC and IEX. 4 A.U. β-membrane lysate (β-Mem) and 4 A.U. pooled antigenic SEC fractions 60-62 (SEC). Remaining lanes contain 4 A.U. of the peak antigenic IEX fraction 21 and identical sample sizes (<4 A.U.) of the neighboring IEX fractions 19, 20, 22 and 23.

FIG. 6D: Purification table for the overall enrichment of antigen.

FIG. 6E. Mass spectrometric analysis (IonTrap) of highly purified antigenic IEX fraction 21 and neighboring fractions that contain an overall smaller amount of antigen (fractions 19, 20, 22 and 23). The summarized spectral intensity of the individual peptides identified is an indicator for the relative abundance of a specific protein in a fraction. Peptides were analyzed using LC/MS/MS (ETD/CID ion trap with HPLC-Chip interface, Agilent Technologies) in the NJMRC Proteomics Facility. Data was searched using the Spectrum Mill search engine (Rev A.03.01.037 SR1, Agilent Technologies, Palo Alto, Calif.).

FIG. 7 presents exemplary data of representative purified peptides showing the best antigenicity using mass spectrometric analysis of IEX fractions.

FIG. 7A: Proteins identified in each fraction following database searching. The summarized numeric spectral intensity of the individual peptides identified is an indicator for the relative abundance of a specific protein in a fraction. Darker colors indicate higher intensity. MS/MS Search scores (far left column) greater than 20 are considered significant.

FIG. 7B: Representative ion trap mass spectra matching for the ChgA peptide AEDQELESLSAIEAELEK (SEQ ID NO: 42). Peptide sequence is shown at the top and band y-ions matching individual fragments are indicated in the mass spectra.

FIG. 7C: Representative ion trap mass spectra matching for the ChgA peptide SDFEEKKEEEGSAN (SEQ ID NO: 43). Peptide sequence is shown at the top and b-ions and y-ions matching individual fragments are indicated in the mass spectra.

FIG. 7D: One embodiment of a ChgA sequence identifying the four peptides that were detected and matched as ChgA antigens (underlined).

FIG. 8 presents one embodiment of a peptide mimotope amino acid sequence, HRPIWARMD (SEQ ID NO: 33), which is one of several mimotopes (Yoshida et al, Intern Immunol 2002) highly stimulatory for BDC-2.5. Chromogranin A is the only protein from the mass spectrometric analysis in FIGS. 6 and 7 that contains sequence homology to the peptide mimotope. WE14, a 14-amino acid sequence from chromogranin A, is a naturally occurring cleavage product of this protein.

FIG. 9 present embodiments of enzymatic conversion of the WE14 peptides and related peptide sequences from chromogranin A through treatment with the enzyme transglutaminase render these sequences highly antigenic for the T cell clone BDC-2.5 and possibly for the other two clones (BDC-10.1 and BDC-5.10.3) sharing reactivity to BDC-2.5 mimotopes.

FIG. 9A: Response of the T cell clone BDC-2.5 to different assay concentrations of β-membrane (blue, Mem) and WE14 peptide (red, WE14). The antigen response is calculated as a percentage of maximal IFN-γ response at 100 mg/ml β-membrane [% Max].

FIG. 9B: Responses of different T cell clones (BDC-2.5, BDC-5.10.3, BDC-10.1, PD-12.4.4 and BDC 5.2.9) to 100 μg/ml WE14 peptide.

FIG. 9C: T cell clone BDC-2.5 activation by various chromogranin A derived peptides (100 μg/ml) expressed as percent response of a control beta membrane preparation.

FIG. 10 presents embodiments of post-translational enzymatic modifications of the WE14 peptides and related peptide sequences from chromogranin A through treatment with the enzyme transglutaminase render these sequences highly antigenic for the T cell clone BDC-2.5 and possibly for the other two clones (BDC-10.1 and BDC-5.10.3) sharing reactivity to BDC-2.5 mimotopes. Enzymatic conversion renders the peptide WE14 highly antigenic for clone BDC-2.5. The WE14 peptide, which is normally only a weak stimulator of the T cell clone BDC-2.5, is converted to a highly antigenic peptide after treatment with a post translational modification enzyme such as transglutaminase. Enz: WE14 after transglutaminase modification. β-Mem: Preparation of β-islet membranes as described herein. WE14: Naturally occurring chromogranin A fragment.

FIG. 11 presents embodiments of post translational enzymatic modifications (PTM) of WE14-related peptides that generate improved antigenicity. (+)=antigenicity. (−)=no antigenicity.

FIG. 12 presents exemplary data showing IFNγ responses (ng/ml) of beta cell membranes to BDC-2.5, BDC-10.1, BDC-5.10.3 and PD-12.4.4 T cell clones from ChgA^{-/-} and ChgA^{+/+} mice.

FIG. 12A: Various concentrations of beta cell tumor membrane proteins

FIG. 12B: Various numbers of islet cells obtained from ChgA^{-/-} mice (red) and control ChgA^{+/+} mice (blue).

FIG. 12C: Summary bar chart of data in FIG. 12B presented as the average concentration of antigen in the islet cells. ChgA^{-/-} (red bars); ChgA^{+/+} (blue bars). Data expressed as antigen units per 10³ islet cells, wherein one unit of antigen is defined as the amount required to induce the production of 10 ng/ml of IFNγ. BDC-2.5 and PD-14.4.4, N=4. BDC-10.1 N=2. BDC-5.10.3=1. Error bars are SEM.

FIG. 13 presents exemplary data of mimotope peptide antigens for the BDC T cells providing a basis for a possible ChgA region encoding an epitope for the BDC T cells.

FIG. 13A: Random mutational design scheme of a baculovirus-encoded library of peptides bound to IA^{g7}.

FIG. 13B: Use of a fluorescent, oligomerized, soluble BDC-2.5 TCR to enrich from the library a virus encoding an IA^{g7}-mimotope (i.e., for example, pS3) that forms a strong ligand with a BDC-2.5 TCR.

FIG. 13C: Three BDC T cell hybridomas stimulated in culture either with: i) immobilized H597 anti-TCR Cβ Mab; ii) ICAM/B7 expressing SF9 cells infected with virus encoding IA^{g7} with a HEL peptide; or ii) ICAM/B7 expressing SF9 cells infected with virus encoding IA^{g7} with pS3. IL-2 production was assayed after 24 hrs.

FIG. 13D: Sequence and activity of pS3-derived mimotopes were compared to those previously identified using other library techniques. Judkowski et al., “Identification of MHC class II-restricted peptide ligands, including a glutamic acid decarboxylase 65 sequence, that stimulate diabetogenic T cells from transgenic BDC2.5 non-obese diabetic mice” J Immunol 166:908-17 (2001); and Yoshida et al., “Evidence for shared recognition of a peptide ligand by a diverse panel of non-obese diabetic mice-derived, islet-specific, diabetogenic T cell clones” Int Immunol 14: 1439-47 (2002). The reported potency of the mimotopes in stimulating the 3 BDC T cell clones is represented qualitatively: ++, very strong stimulation; +, modest stimulation; −, little or no stimulation. The striking motif at p5, p7, p8 is highlighted in red.

FIG. 13E: IFNγ production from BDC-2.5 and BDC-10.1 T cell clones using ICAM/B7 SF9 cells were infected with Baculovirus cultures encoding membrane-anchored IA^{g7} covalently bound to either: i) pHEL; ii) pS3; or iii) WEDKRWSRMD (SEQ ID NO: 44).

FIG. 13F: The p3 glycine of pS3 was mutated to other amino acids. The effect of the mutations on early activation of the three BDC hybridomas was assessed by CD69 induction. The results are shown as the percent of cells expressing CD69 relative to those activated with the unmutated pS3 peptide. Some amino acids (Ala, Ser, Thr) had little effect (open bars), while others (Lys, Trp, Glu, Ile) virtually eliminated activation of all three clones (filled bars). The sequences of the pS3 and ChgA peptide are also shown, highlighting the amino acids at the p3 position.

FIG. 14 presents one embodiment of a ChgA-derived peptide (WE14) and exemplary data showing activation of three BDC T cells.

FIG. 14A: A portion of the chromogranin A (ChgA) amino acid sequence with the WE14 peptide indicated by the arrows. Putative positions in the IA^{g7} peptide-binding groove (i.e., for example, p1-p9) are shown and a motif common to some antigen peptide mimotopes is highlighted in red.

FIG. 14B: IFNγ response (ng/ml) of the BDC-2.5, BDC-10.1, BDC-5.10.3 and PD-12.4.4 T cell clones stimulated by various concentrations of pS3 (green), WE14 (red), INS2 B9-23 (SHLVEALYLVCGERG (SEQ ID NO: 45)) (magenta) and beta cell tumor membrane preparation (β-Mem) (blue). The data represents the average values measured from at least two separate experiments.

FIG. 15 presents exemplary data showing processing of the WE14 peptide that results in optimal presentation by IA^{g7}.

FIG. 15A: IFNγ response of the BCD-2.5 T cell clone to varying concentrations (5-500 μM) of ChgA-derived peptides. Data are representative of two separate experiments.

FIG. 15B: A series of ChgA peptides tested for their ability to compete with a biotinylated HEL peptide for binding to soluble IA^{g7}. pS3-positive control peptide. IE^{k} moth cytochrome c-negative control peptide. N=2. Y axis: Percent of IA^{g7}-bound biotinylated HEL peptide as compared to IE^{k}. X axis: Log concentration of inhibitor peptide.

FIG. 15C: A multiple regression analysis of the set of parallel polynomial inhibition curves shown in FIG. 15A and FIG. 15B. The results are presented as the stimulatory or inhibitory activity of the peptides relative to WE14.

FIG. 16 presents exemplary data showing that the immunization of NOD T cell receptor transgenic (TCR-Tg) mice with the WE14 peptide sequence (WSRMDQLAKELTAE (SEQ ID NO: 11)) suppresses the inflammatory response of diabetogenic T cells in the BDC-2.5 TCR-Tg mouse.

FIG. 17 presents exemplary data showing that the immunization of NOD mice with the WE14 peptide sequence (WSRMDQLAKELTAE (SEQ ID NO: 11)) suppresses the inflammatory response of primary T cells in the NOD mouse.

FIG. 18 presents exemplary data showing that the adoptive transfer of diabetes to NOD.scid (NOD mice immunodeficient in T or B lymphocytes) recipients is delayed if donor T cells are from NOD mice immunized with the WE14 peptide sequence (WSRMDQLAKELTAE (SEQ ID NO: 11)).

### DETAILED DESCRIPTION

The present invention is related to the development and treatment of autoimmune disease. Autoimmune diseases can result from tissue damage caused by the activation of autoreactive T cells by autoantigens. For example, fragments of naturally occurring proteins (i.e., for example, chromogranin A) may activate autoreactive T cells that result in the destruction of pancreatic β islet cells. Inhibition of autoantigen-autoreactive T cell binding may provide therapeutic as well a prophylactic treatments for autoimmune diseases.

In one embodiment, the present invention contemplates a set of antigenic peptides derived from the chromogranin A secretory peptide. In one embodiment, the antigenic peptides may result in vivo from enzymatic post-translational modifications of the chromogranin A peptide. Although it is not necessary to understand the mechanism of an invention, it is believed that these antigenic chromogranin A peptides induce an autoreactive T cell response and may be responsible for the initiation and development of autoimmune-induced Type 1 diabetes by, for example, the release of inflammatory cytokines (i.e., for example, interferon-γ).

The data presented herein shows that chromogranin A was identified as a putative autoantigen after ion exclusion chromatography and/or high performance liquid chromatography of beta cell adenoma tissue preparations, fragmentation into peptides by tryptic digestion, and mass spectrometry analysis. Other potential autoantigen candidates included secretogranins 1 and 2, insulin-2, and insulin-like growth factor II. However, only chromogranin A autoantigen peptides contained a sequence EDKRWSRMD (SEQ ID NO: 46) with homology to the peptide mimotopes HRPIWARMD (SEQ ID NO: 33) and HIPIWARMD (SEQ ID NO: 36) that activated a panel of diabetogenic CD4+ Th1 T cell clones (i.e., for example, BDC-2.5 or BDC-10.1).

In one embodiment, the present invention contemplates at least one chromogranin A variant. In one embodiment, the variant comprises a natural cleavage product of chromogranin A. In one embodiment, the cleavage product comprises the amino acid sequence WSRMDQLAKELTAE (SEQ ID NO: 11); (WE14). In one embodiment, the WE14 variant comprises at least one additional N-terminal amino acid. Although it is not necessary to understand the mechanism of an invention, it is believed that while WE14 is a very weak autoantigen to a T cell clone (i.e., for example, BDC-2.5), enzymatic conversion of this peptide, and longer peptides containing this sequence, results in significantly increased antigenic efficacy. Such enzymatic reactions may occur in vivo as a result of post-translational modification and include, but are not limited to, hydrolysis, deamidation, or peptide multimerization.

### I. Chromogranin A

Chromogranin A (ChgA) is widely expressed in neuroendocrine tissue and a cleavage product (i.e., for example, WE14) has been described not only in pancreatic islet beta cells, but also in other gastro-entero-pancreatic tissues such as the adrenal gland. Gleeson et al., “Occurrence of WE-14 and chromogranin A-derived peptides in tissues of the human and bovine gastro-entero-pancreatic system and in human neuroendocrine neoplasia” J Endocrinol 151:409-420 (1996). It is unclear why an autoimmune attack by ChgA antigens on tissues other than the pancreas has not been observed. One possibility is that the potency of pancreatic ChgA antigens might be dependent on a pancreas-specific post-translational modifications. Alternatively, selective destruction of pancreatic beta cells in pancreatic islets has been attributed to their high sensitivity to inflammatory damage compared to other islet cells. Mathews et al., “Mechanisms underlying resistance of pancreatic islets from ALR/Lt mice to cytokine-induced destruction” J Immunol 175: 1248-1256 (2005). On the other hand, other neuroendocrine cells may be more resistant to, or protected from, ChgA antigen mediated immune damage.

The question arises as to why T cells specific for ChgA exist, given that the widespread expression of this protein might be expected to result in efficient deletion of ChgA-specific T cells during thymic development. In addition to tissue or inflammation specific processing or post-translational modifications, another possibility may be poor thymic expression. Modulation of thymus medullary epithelium RNA expression by the AIRE gene failed to express ChgA RNA under any circumstances. Anderson et al., “Projection of an immunological self shadow within the thymus by the AIRE protein” Science 298:1395-1401 (2002). This report suggests that here may not be sufficient ChgA in the thymus to mediate deletion.

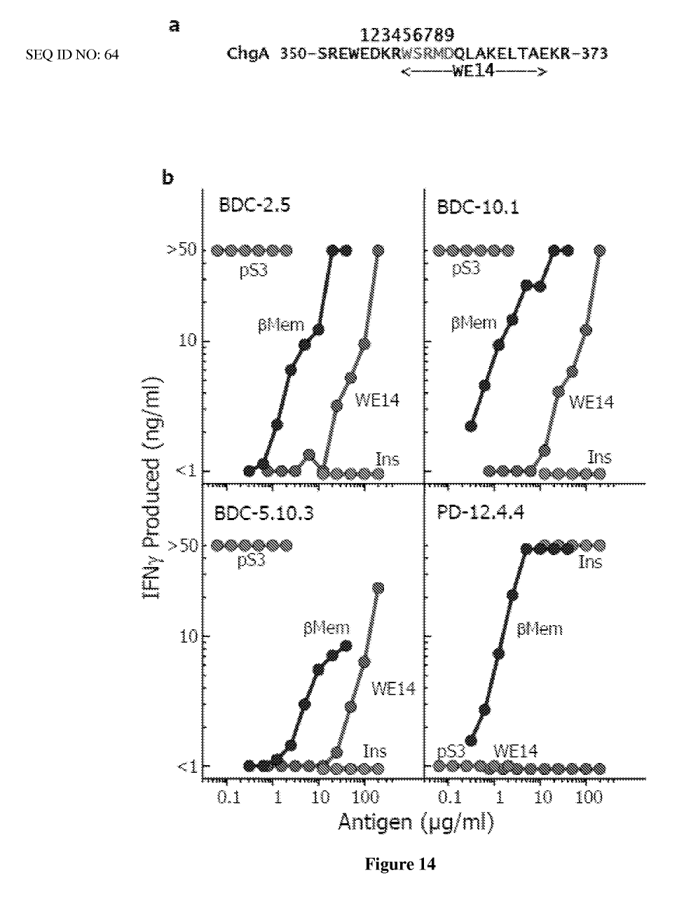

The identification herein that WE14 as an active ChgA peptide was most surprising. For example, it was most unexpected that WE14's WSRMD (SEQ ID NO: 52) motif variant would fail to fill all amino acid positions in the IA^{g7}-binding groove prior to p5 as was predicted by the mimotope study (infra). One would expect that peptides that do not properly fit the IA^{g7}-binding groove would be predicted to bind very poorly, if at all, to IA^{g7}. Not only because of the lack of the major p1 and p4 anchor amino acids, but also because a number of normally highly conserved H-bonds to the peptide backbone would be missing. Nevertheless, the data presented herein show that the WE14 peptide can bind to IA^{g7} and stimulate all three of the BDC clones. FIGS. 14 & 15.

A T cell peptide epitope that does not fill the MHCII groove is not unprecedented in autoimmunity. In the mouse model of EAE, the N-terminal peptide of myelin basic protein is a major T cell epitope, but structural studies have concluded that the natural form of this peptide that is recognized by T cells does not fill the beginning of the IA^{u} binding groove. Maynard et al., “Structure of an autoimmune T cell receptor complexed with class II peptide-MHC: insights into MHC bias and antigen specificity” Immunity 22:81-92 (2005). He et al., “Structural snapshot of aberrant antigen presentation linked to autoimmunity: the immunodominant epitope of MBP complexed with I—Au” Immunity 17:83-94 (2002). In these studies, an active variant of the peptide that filled the rest of the groove with small amino acids was used. In the structure of a T cell receptor (TCR) bound to this complex, relatively little contact was made to the extra peptide amino acids.

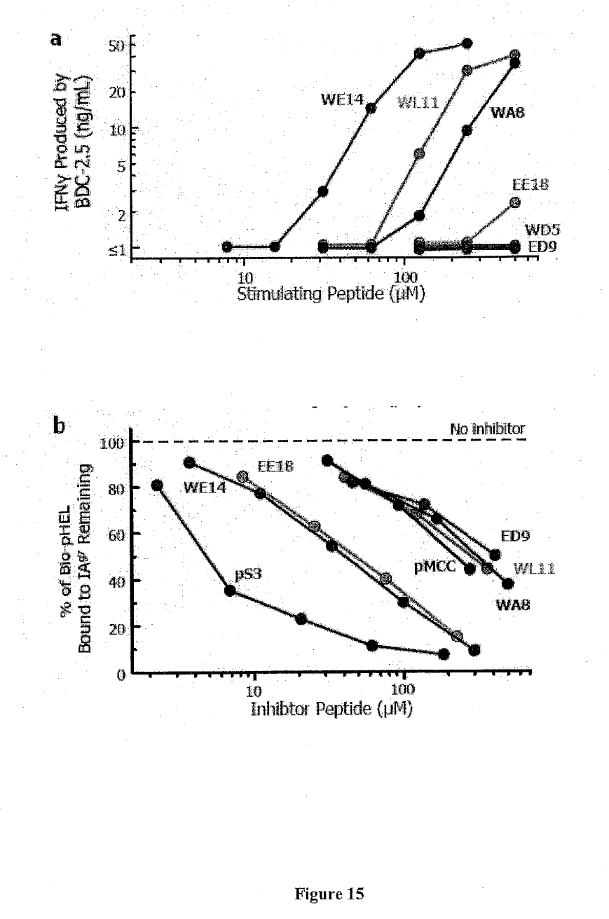

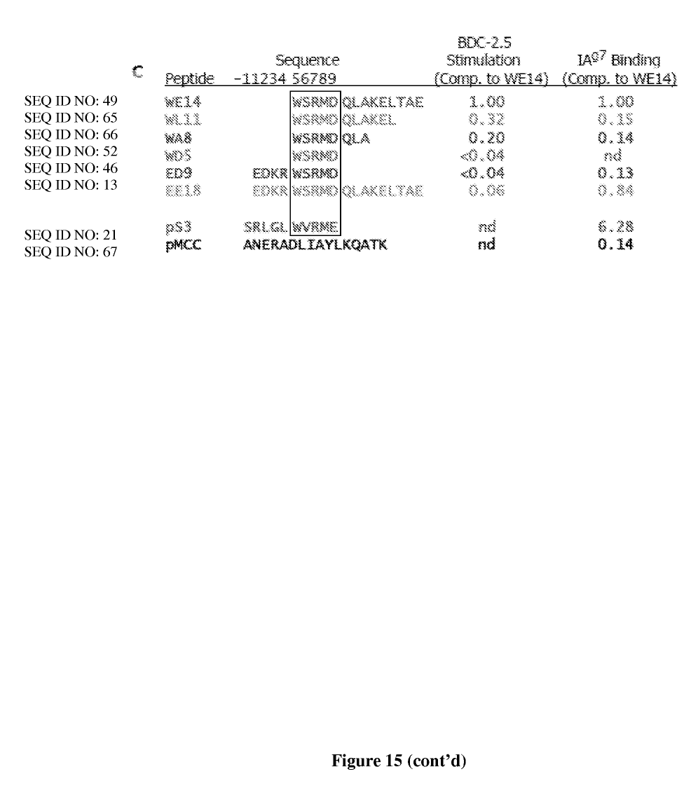

Although it is not necessary to understand the mechanism of an invention, it is believed that a C-terminal nine (9) amino acid sequence plays a role in binding to IA^{g7} whereas truncation of even the last 3 amino acids of the peptide severely reduces IA^{g7} binding and diminishes the T cell response. See, FIG. 15B and FIG. 15C. These data strongly suggest that the C-terminus of WE14 might interact with IA^{g7} at a site outside of the normal peptide binding groove, compensating for the lack of the p1-p4 portion of the peptide. Furthermore, extending the N terminus of the WE14 peptide (i.e., for example, WD5) inhibited, rather than enhanced, peptide presentation and was not able to restore IA^{g7} binding or T cell activation to the WE14 version of the shared WSRMD (SEQ ID NO: 52) motif.

### II. Autoimmune Disease

An autoimmune disorder is a condition that occurs when the immune system mistakenly attacks and destroys healthy body tissue. There are more than 80 different types of autoimmune disorders. Normally the immune system's white blood cells helps protect the body from harmful substances, called antigens. Examples of antigens include bacteria, viruses, toxins, cancer cells, and foreign blood or tissues from another person or species. The immune system produces antibodies that destroy these harmful substances. But in patients with an autoimmune disorder, the immune system can't tell the difference between healthy body tissue and antigens. The result is an immune response that destroys normal body tissues. The response is a hypersensitivity reaction similar to allergies, where the immune system reacts to a substance that it normally would ignore. In allergies, the immune system reacts to an external substance that would normally be harmless. With autoimmune disorders, the immune system reacts to normal body tissues.

What causes the immune system to no longer distinguish between healthy body tissues and antigens is unknown. One theory holds that various microorganisms and drugs may trigger some of these changes, particularly in persons who are genetically prone to autoimmune disorders. An autoimmune disorder may result in: i) the destruction of one or more types of body tissue; ii) abnormal growth of an organ; or iii) changes in organ function. An autoimmune disorder may affect one or more organ or tissue types. Organs and tissues commonly affected by autoimmune disorders include, but are not limited to, red blood cells, blood vessels, connective tissues, endocrine glands such as the thyroid or pancreas, muscles, joints, or skin.

A person may have more than one autoimmune disorder at the same time. Examples of autoimmune (or autoimmune-related) disorders include but are not limited to, Hashimoto's thyroiditis, pernicious anemia, Addison's disease, type I diabetes, rheumatoid arthritis, systemic lupus erythematosus, dermatomyositis, Sjogren syndrome, lupus erythematosus, multiple sclerosis, myasthenia gravis, reactive arthritis, Grave's disease, or celiac disease.

In general, symptoms of an autoimmune disease may include, but are not limited to, dizziness, fatigue, general ill-feeling, or low-grade fever. While each disease is highly specific initial diagnostic tests may include erythrocyte sedimentation rate (ESR) or C-reactive protein (CRP).

The goals of treatment are to reduce symptoms and control the autoimmune process while maintaining the body's ability to fight disease. Treatments vary widely and depend on the specific disease and your symptoms. The outcome depends on the specific disease. Most are chronic, but many can be controlled with treatment.

Self-antigen targets in many autoimmune diseases for both humans and mice can be identified by detecting serum autoantibodies. For example, in autoimmune disease such as systemic lupus erythematosis (SLE), immunoglobulin in rheumatoid arthritis (RA), and insulin in type I diabetes (T1D) DNA and chromatin may comprise self-antigens. Most autoimmune diseases also involve autoreactive CD4 T cells which are required for autoantibody production and can also be pathogenic as in T1D, but identifying the relevant T cell autoantigen epitopes has been much more difficult. In some cases, epitopes for autoreactive CD4 T cells have been found in the same proteins targeted by autoantibodies. One such example is insulin, which is targeted by both autoreactive CD4 T cells and autoantibodies in mice and humans. In most cases, however, the targets of important autoreactive T cells have remained undefined.

There appears to be considerable overlap between mouse and human autoantigens that mediate several autoimmune diseases including, but not limited to, multiple sclerosis (i.e., for example, myelin basic protein), rheumatoid arthritis (i.e., for example, collagen) and lupus erythematosis (i.e., for example, DNA and chromatin), as well as T1D (i.e., for example, insulin). As similar situation may exist for ChgA autoantigens wherein human WE14 peptide has a sequence that is nearly identical to that of mouse. Curry et al., “Isolation and primary structure of a novel chromogranin A-derived peptide, WE-14, from a human midgut carcinoid tumour” FEBS Lett 301:319-21 (1992). Furthermore, the similarity in binding and presentation of peptides between IA^{g7} and the human DQ alleles associated with T1D32 suggests that WE14 may be presented by MHCII in T1D susceptible humans.

### III. Diabetes

Diabetes is a chronic (lifelong) disease marked by high levels of sugar in the blood. Insulin is a hormone produced by the pancreas to control blood sugar. Diabetes can be caused by too little insulin, resistance to insulin, or both. One underlying mechanism regarding diabetes involves abnormal digestion, absorption and metabolism of glucose. Glucose is a source of fuel for the body and is controlled by insulin from the pancreas. The role of insulin is to move glucose from the bloodstream into muscle, fat, and liver cells, where it can be used as fuel. People with diabetes have high blood sugar. This is because their pancreas does not make enough insulin; and/or their muscle, fat, and liver cells do not respond to insulin normally.

A. Clinical Characteristics

There are three major types of diabetes: Type 1 diabetes is usually diagnosed in childhood. Many patients are diagnosed when they are older than age 20. In this disease, the body makes little or no insulin. Daily injections of insulin are needed. The exact cause is unknown. Genetics, viruses, and autoimmune problems may play a role. Type 2 diabetes is far more common than type 1. It makes up most of diabetes cases. It usually occurs in adulthood, but young people are increasingly being diagnosed with this disease. The pancreas does not make enough insulin to keep blood glucose levels normal, often because the body does not respond well to insulin. Many people with type 2 diabetes do not know they have it, although it is a serious condition. Type 2 diabetes is becoming more common due to increasing obesity and failure to exercise. Gestational diabetes is high blood glucose that develops at any time during pregnancy in a woman who does not have diabetes. Diabetes affects more than 20 million Americans. Over 40 million Americans have prediabetes.

There are many risk factors for type 2 diabetes, including, but not limited to, age over 45 years, family history, gestational diabetes or delivering a baby weighing more than 9 pounds, heart disease, high blood cholesterol level, obesity, lack of exercise, polycystic ovary disease, impaired glucose tolerance, ethnicity (particularly African Americans, Native Americans, Asians, Pacific Islanders, and Hispanic Americans).

In general, diabetes symptoms include, but are not limited to, high blood levels of glucose, blurry vision, excessive thirst, fatigue, frequent urination, hunger, or weight loss Type 1 diabetes symptom include, but are not limited to, high blood levels of glucose, fatigue, increased thirst, increased urination, nausea, vomiting, or weight loss in spite of increased appetite.

Conventional diagnostic examinations and testing include, urine analysis to look for glucose and ketones from the breakdown of fat. However, a urine test alone does not diagnose diabetes. Other tests are used to diagnose diabetes including: fasting blood glucose level—diabetes is diagnosed if higher than 126 mg/dL on two occasions. Levels between 100 and 126 mg/dL are referred to as impaired fasting glucose or pre-diabetes. These levels are considered to be risk factors for type 2 diabetes and its complications; oral glucose tolerance test—diabetes is diagnosed if glucose level is higher than 200 mg/dL after 2 hours. (This test is used more for type 2 diabetes.); random (non-fasting) blood glucose level—diabetes is suspected if higher than 200 mg/dL and accompanied by the classic diabetes symptoms of increased thirst, urination, and fatigue. (This test must be confirmed with a fasting blood glucose test.). Alternatively, a hemoglobin A1c (HbA1c) level may be checked every 3-6 months. The HbA1c is a measure of average blood glucose during the previous 2-3 months.

It is generally accepted that Type 1 Diabetes (T1D) results from a breakdown in tolerance to multiple β-cell proteins, with a consequent immune-mediated destruction of these insulin producing cells (1). Over the past 20 years considerable progress has been made towards identifying those members of the population most at risk of developing T1D through the combination of family studies, MHC haplotyping and the measurement of circulating autoantibodies (2-4). In spite of this familial association, the majority of newly diagnosed T1D individuals still come from outside the defined “high-risk” category (5, 6). This is, in part, not only because few autoantigens have been identified, but also because humoral assays are only surrogate markers for pancreatic islet pathogenic events (i.e., for example, autoreactive T cell-mediated cell destruction). While MHC-peptide tetramers, might be capable of directly measuring the presence or absence of diabetogenic autoreactive T-cells, to enhance diagnostic performance, peptide epitopes recognized by these autoreactive T-cells are not well known.

Presently, it is possible to identify a significant percentage of individuals at high risk of developing T1D within a 10-year time frame, only minimal success has been achieved towards developing effective therapeutic strategies. In one embodiment, the present invention contemplates effective therapeutic strategies that can either prevent or delay disease occurrence in prediabetic subjects, or prevent recurrent autoimmune attack following transplantation of pancreatic islets to diabetic patients, without continuous immunosuppression.

Anti-CD3 therapy is believed by some to suggest an effective regimen, but trial results suggest that more sophisticated, antigen-specific reagents will likely be required (7, 8). Thus, it appears that during an autoimmune disease, the number of involved autoantigens increase as inflammatory damage to tissue proceeds. In regards to diabetes, little is understood about the significance of the totality of autoantigens and their individual roles in disease. Although it is not necessary to understand the mechanism of an invention, it is believed that tolerance induction to one or more specific autoantigens may provide an effective therapeutic intervention.

Nonetheless, it is also believed that effective therapies may also include specific autoantigens to which a specific patient may have already demonstrated reactivity, or a prophylactic approach to autoimmune responses that have not yet been generated. Selecting the right therapeutic intervention for the right patient at the right time, therefore involves a complete understanding of the number, identity, and relationship of potential autoantigens. In particular, it has been shown that an autoantigen appearing in a first individual may appear at an earlier or later time point (or not at all) in a second individual (2).

Thus, the present invention contemplates methods and compositions demonstrating that the limited number of identified autoimmune autoantigens are insufficient to provide proper therapeutic and prophylactic regimes for all susceptible members of the human population. Accordingly, it is believed that, in the case of autoimmune mediated T1D, a characterization of all potential T1D autoantigens will provide useful and effective regimens for the human population.

Pancreatic peptides have been unambiguously identified using a combination of mass spectrometry and high pressure liquid chromatography in a effort to identify pancreatic peptidomes (i.e., spatial and temporal peptide expression patterns). Boonen et al., “Neuropeptides of the islets of Langerhans: peptidomics study” Gen Comp Endocrinol 152:231-241 (2007). This technique may contribute to the treatment of diabetes by successfully localizing chromogranins A, B, and C and the WE14 protein within a tissue, it is not useful to identify autoantigens that induce autoreactive T cells.

B. Diabetic NOD Mouse Model

The NOD mouse model of T1D can provide a population of pathogenic CD4 T cells for either in vitro or in vivo experimentations. A series of studies have identified CD4 T cells in NOD mice that are not only reactive with in vitro pancreatic antigens but also cause and/or accelerate in vivo diabetes development. Some of these clones have turned out to be specific for insulin epitopes. However, the antigenic targets of other highly pathogenic CD4 T cell clones (i.e., for example, the BDC clones, including, but not limited to, the BDC-2.5 clone), isolated from the spleens and lymph nodes of diabetic NOD mice have not been identified. Haskins et al., “Pancreatic islet specific T-cell clones from non-obese diabetic mice” Proc Natl Acad Sci USA 86:8000-8004 (1989); and Haskins K., “Pathogenic T-cell clones in autoimmune diabetes: more lessons from the NOD mouse” Adv Immunol 87:123-62 (2005). The BDC clones are not responsive to insulin, but respond to pancreatic islet cells or cell extracts from beta cell adenomas in the presence of IA^{g7}-bearing antigen-presenting cells in vitro. Bergman et al., “Islet-specific T-cell clones from the NOD mouse respond to beta-granule antigen” Diabetes 43:197-203 (1994); and Bergman et al., “Biochemical characterization of a beta cell membrane fraction antigenic for autoreactive T cell clones” J Autoimmun 14:343-51 (2000). These studies also suggest that the majority of these clones react to a common, but unidentified, pancreatic antigen. The highly pathogenic nature of the BDC clones has been demonstrated by adoptive transfer studies into young NOD mice in which the development of T1D is greatly accelerated. Haskins, K., “Pathogenic T-cell clones in autoimmune diabetes: more lessons from the NOD mouse” Adv Immunol 87:123-62 (2005); and Haskins et al., “Acceleration of diabetes in young NOD mice with a CD4+ islet-specific T cell clone” Science 249:1433-1436 (1990). Further, T cells from BDC T cell receptor (TCR) transgenic mice are similarly aggressive in vivo. Katz, J. D., Wang, B., Haskins, K., Benoist, C. & Mathis, D. Following a diabetogenic T cell from genesis through pathogenesis. Cell 74, 1089-100 (1993); and Pauza et al., “T-Cell Receptor Transgenic Response to an Endogenous Polymorphic Autoantigen Determines Susceptibility to Diabetes” Diabetes 53:978-988 (2004). In addition, introduction of BDC TCR genes into T cell deficient NOD.scid mice (retrogenic mice) rapidly induces T1D. Burton et al., “On the pathogenicity of autoantigen-specific T-cell receptors” Diabetes 57: 1321-1330 (2008).

### IV. Autoreactive T Cells

The present invention uses the strategy of proteomics to identify and define autoimmune autoantigens (i.e., for example, directed to diabetogenic T cells). Although it is believed that the presence of an antibody directed to an autoantigen suggests a corresponding reactivity of a T cell, not all T cell reactivities will generate autoantibodies by inducing B cells. For example, some T cells may react to autoantigens by releasing inflammatory cytokines (i.e., for example, interferon-γ) that may play a role in the development and maintenance of autoimmune diseases (i.e., for example, type 1 and/or type 2 diabetes). Identification of autoreactive T cell antigens thus requires an approach that goes beyond the classic procedure of identifying antigenic targets through antibody recognition (i.e., for example, autoreactive T cell antigens can be defined by their ability to stimulate T cell function). Although it is not necessary to understand the mechanism of an invention, it is believed that autoreactive T cell activation by an autoantigen involves a presentation of the autoantigen by an antigen-presenting cell (APC), as opposed to a direct interaction between a T cell receptor and an autoantigen.

Autoreactive T-cells are believed to be mediators in the initiation and propagation of the autoimmune disease process. In one embodiment, the present invention contemplates a method for measuring T cell responses to ChgA peptides in human patients. In one embodiment, the T cell response comprises a T cell activation. Although it is not necessary to understand the mechanism of an invention, it is believed that such T cell activation identifies ChgA peptides as new biomarkers of autoimmune diseases such that ChgA peptide epitopes are useful in tolerizing regimes. In one embodiment, the method further comprises measuring, in a human biological sample, an increased number of human T cells having specificity for ChgA peptide epitopes. In one embodiment, the biological sample may including but not limited to, a blood sample or a tissue sample. In one embodiment, the blood sample may include, but not limited to, a whole blood sample, a plasma sample, or a sera sample. In one embodiment, the tissue sample may include, but not limited to, a pancreas tissue sample, an thymus sample, or a lymph node sample

1. Diabetes Autoreactive T Cells

Recent efforts to identify T cell autoantigens in T1D, to which a humoral response is not evident, have primarily been directed towards screening peptide libraries that are based upon the consensus binding motifs of appropriate MHC molecules. These techniques have identified mimotopes for several T-cells, including the BDC-2.5 clone (9, 10).

These studies are inconclusive, however, because the promiscuity of an APC-peptide-T cell interaction makes it virtually impossible to identify native targets from peptide display results. Expression cloning of pancreatic β-cell derived cDNA libraries in either mammalian or bacterial cells are capable of yielding more interpretable results. For example, insulin B15-23 was identified as a natural ligand of a diabetogenic CD8+ T cell clone by expression cloning (11). However expression cloning has other disadvantages as the technique is greatly influenced by the size and abundance of the relevant cDNA in the library, and incorporate the inherent difficulties usually encountered during MHC class II-restricted epitope research.

Consequently, a direct study of native tissue would appear to be the optimal investigative approach to identify autoantigens. But, until just recently, there has been little success in attempts to identify the natural origin of diabetogenic T cell peptides by directly measuring T cell stimulation to APCs exposed to complex antigenic mixtures. For example, a recent report identified a β-cell antigen targeted by pathogenic CD8+ T cells through a proteomics approach (12). Cellular fractions were obtained for testing with a cytolytic T cell clone following chromatographic separation of peptides eluted from the H-2 Kd molecules purified from the pancreatic β-cell line NIT-1. Analysis by mass spectrometry showed major peak components derived from a ligand for the T cell clone. Subsequent protein database searches led to an exact match between the T cell clone ligand and a murine islet-specific glucose-6-phosphatase catalytic subunit-related protein (IGRP) (12). Proteomic methods are utilized herein in a highly focused manner to identify proteins within purified β-cell membrane fractions believed to contain autoantigens reactive with a panel of CD4+ class II-restricted, diabetogenic T cell clones isolated from the NOD mouse.

2. Diabetogenic T Cell Clones

Reagents available for the detection of T cell antigens are believed limited because the number of well-characterized diabetogenic T cell clones is quite small. The BDC collection of CD4+ T cell clones (i.e., for example, BDC-2.5) are highly active in the acceleration or induction of in vivo diabetes models. However, their usefulness has been somewhat limited because their antigenic target was not known. The data presented herein identifies one source of antigens for a major cohort of these clones. One of these antigen sources comprise a ChgA protein. The ChgA protein is usually found in the secretory granules of pancreatic beta cells and other neuroendocrine tissues. Gleeson et al., “Occurrence of WE-14 and chromogranin A-derived peptides in tissues of the human and bovine gastro-entero-pancreatic system and in human neuroendocrine neoplasia” J Endocrinol 151:409-20 (1996); and Curry et al., “Chromogranin A and its derived peptides in the rat and porcine gastro-entero-pancreatic system. Expression, localization, and characterization” Adv Exp Med Biol 482:205-13 (2000). The data also show that a natural 14 amino acid cleavage product of ChgA (WE11), when presented by IA^{g7} APC, activates the ChgA-specific T cell clones in vitro. A representative BDC panel of diabetogenic CD4 T cell clones is well documented. (13-16); Table 1.

TABLE 1
Diabetogenic CD4+ Th1 T cell clones

| | | | Diabetogenicity | |
|---|---|---|---|---|
| | | | NOD | NOD.scid |
| Clone | TCR | Islet Ag Reactivity | (<14 d) | (<14 d.) |
| BDC-2.5 | Vb4Va1 | All mouse strains | + | + |
| | | tested* | | |
| BDC-4.12 | Vb19Va(nd) | ″ | + | n.d. |
| BDC-5.2.9 | Vb6Va12 | Mouse, rat | + | + |
| BDC-5.10.3 | Vb4Va1 | All mouse strains tested | + | + |
| BDC-6.3 | Vb4Va3.1 | ″ | + | − |
| BDC-6.9 | Vb4Va13.1 | NOD, SWR | + | + |
| BDC-9.3 | Vb4Va13.1 | NOD, SWR | + | + |
| BDC-10.1 | Vb15Va13 | All mouse strains tested | + | + |

*All mouse strains tested: NOD, NOR, BALB/c, CBA, C57BL/6, C57L/J, SWR, SJL

One of these T cell clones, BDC-2.5 comprises a BDC-2.5 TCR that was used to make the 2.5 TCR transgenic (Tg) mouse. (17). Another TCR-Tg mouse was made from a second clone in the panel, BDC-6.9, and exists in a NOD congenic lacking the antigen (18). The properties of these and other NOD-derived T cell clones, as well as the TCR-Tg mice that have been generated from them, were described in detail in a recent review. (19). Distinguishing features of these clones comprise the display of a CD4 Th1 T cell phenotype and exhibit diabetogenic activity in vivo.

More recent work, obtained from an ex vivo analysis of T cells retrieved after adoptive transfer, suggests that a variety of inflammatory cytokines and chemokines may be produced after diabetogenic CD4 T cell migration to the pancreas. Consequently, these T cells promote the recruitment and activation of inflammatory macrophages into the site. (20; Cantor, J. and Haskins, K., “Recruitment and activation of macrophages by pathogenic CD4 T cells in T1D: Involvement of CCR8 and CCL1” J. Immunol. 179:5760-5767 (2007)).

In one embodiment, the present invention contemplates a method of identifying β-islet autoantigens capable of activating diabetogenic CD4+ Th1 T cell clones. In one embodiment, the autoantigen activates at least one CD4+ Th1 T cell clone. In one embodiment, at least one clone comprises BDC-2.5.

In one embodiment, the autoantigen activates at least two CD4+ Th1 T cell clones. In one embodiment, the at least two clones comprise BDC-2.5 and BDC-5.10.3. In one embodiment, the at least two clones comprise BDC-6.9 and BDC-9.3. Although it is not necessary to understand the mechanism of an invention, it is believed that these clones may share the same TCR, but as the clones came from different lines (2 and 5, 6 and 9), they also may come from different individual mice, suggesting that the same antigen specificities arise in different animals.

In one embodiment, the autoantigen activates at least three CD4+ Th1 T cell clones. In one embodiment, the autoantigen activates a panel of CD4+ Th1 T cell clones, wherein said panel is selected from the group consisting of those identified in Table 1. In one embodiment, the autoantigen comprises a natural CD4+ Th1 T cell clone ligand.

Specific advantages to the panel embodiments the present invention contemplates includes, but is not limited to: i) it is still not known whether there are specific autoantigens that drive the disease process, particularly in the initial stages; ii) Table 1 reflects a comprehensive listing of diabetogenic T cell clones available; and iii) all of the clones listed in Table 1 react to some entity contained within a beta cell membrane fraction. Although it is not necessary to understand the mechanism of an invention, it is believed that a beta cell membrane faction may possibly point towards a common protein or group of proteins as important autoantigens. (19).

Recent study indicates that there may be a limited number of β-islet-reactive NOD TCR with diabetogenic potential. For example, various strains of TCR retrogenic (Rg) mice were produced from NOD TCRs. Twelve (12) Rg strains were produced from clones with known diabetogenic autoantigens (i.e., for example, GAD65, IA2, phogrin, and insulin), and four (4) Rg strains were produced from TCR clones with an unknown antigen specificity. Of these strains, only a few TCR-Rg mice were shown to have diabetogenic potential. Burton et al., “On the pathogenicity of autoantigen-specific T cell receptors” Diabetes 57:1321-30 (2008). With the exception of one insulin-reactive clone, Rg mice developing diabetes were those comprising a TCR from T cell clones with an unknown specificity, wherein three of the four were T cell clones appearing within the presently presented Table 1. For example, retrogenic mice comprising a TCR from an autoreactive T cell clone selected from the group comprising BDC-2.5, BDC-6.9, or BDC-10.1, all exhibited a high incidence of early diabetes (i.e., for example, diabetogenic). In particular, diabetes was particularly aggressive in BDC-10.1 mice appearing within about one month of age. This pattern of development resembles diabetes in 2.5 TCR-Tg mice on the NOD.scid background. (21). Although it is not necessary to understand the mechanism of an invention, it is believed that these findings emphasize the advantages of using a panel of T cell clones to screen for diabetogenic autoantigens and highlights the importance of identifying their respective antigenic specificities.

C. Isolation of Pancreatic Beta Cell Autoantigens

The data presented herein examines the efficacy of various natural and synthetic autoantigens that are believed to activate autoimmune T cells directed against pancreatic beta cells. Consequently, the various embodiments presented herein were compared against a positive control preparation comprising pancreatic beta cell membranes. Although it is not necessary to understand the mechanism of an invention, it is believed that these beta cell membranes comprise pancreatic beta cell autoantigens. In some embodiments, the beta cell membranes comprise mouse beta cell membranes. In one embodiment, the beta cell membranes comprise human beta cell membranes. In one embodiment, the positive control preparation comprise synthetic human beta cell autoantigens. In some embodiments, a specific amino acid sequence is synthesized using a commercially available protein synthesis institution.

Previous studies have described the biochemical fractionation of enriched islet cell organelles to isolate pancreatic autoantigens. For example, using β-cells from freshly isolated adenomas produced in the transgenic RIP-Tag mouse, various fractions can be prepared from either enriched or deficient in insulin secretory granules. (22). Pancreatic β-cells isolated from RIP-Tag mouse tumors are high in yield and are highly antigenic. Further, these isolated cells can be maintained as antigenic cell lines as conventional cell cultures. The results of these studies indicated that the antigenic activity within the above panel of T cell clones (see, Table 1) was found primarily in the membrane portion of the β-cell granules, and was not a result of any of the previously reported autoreactive proteins (i.e., for example, insulin and GAD). Additional biochemical information about this antigenic membrane fraction has been accumulated and published. (23).

Autoantigens for diabetogenic T cell clones may not have not yet been completely identified because previous attempts at identifying antigens for T cells, particularly class II-restricted T cells, have been hampered by either lack of an appropriate biological system or limitations of the applied technology. As suggested above, currently reported data have failed to identify autoantigens for most diabetogenic T cell clones. For example, one biological system limitation encountered by those in the art is detecting the response of an autoreactive T cell clone. Although autoreactive T cells from some TCR transgenic mice can be used to detect stimulation by peptide ligands, these models are unreliable and inconsistent as a read-out system for antigen responses because the quantitative level of each activation state (i.e., for example, individual responsiveness) vary widely both within and between individual mice. TCR transgenic mouse models are especially unsuitable for detecting very small amounts of antigen in complex protein mixtures such as whole cells or cell lysates.

In one embodiment, the present invention contemplates a method for detecting autoreactive T cell clone responses to autoantigens utilizing at least 500 β-islet cells. In one embodiment, the method utilizes between approximately 500-1000 β-islet cells. In one embodiment, the method utilizes between approximately 1,000-5,000 β-islet cells. In one embodiment, the method utilizes between approximately 10,000-100,000 β-islet cells. Although it is not necessary to understand the mechanism of an invention, it is believed that ˜1×10⁵ β-islet cells is equivalent to approximately 5-10 μg of whole beta cell membrane preparation.

Identification of diabetogenic autoantigens has proved extraordinarily difficult. First and foremost, studies have been severely limited by insufficient quantities of antigenic starting material. Consequently, efforts have been expended on developing proper culture and analysis techniques of beta tumor cell lines. Most researchers, however, found that repeated cell passages during routine culturing of β-cell lines resulted in loss of autoantigen. Alternatively, transgenic NOD RIPTag mice bearing the beta cell adenomas are commercially available, but are not routinely available and difficult to maintain. (24) As a result, active antigenic fractions can be obtained after chromatography of some beta cell lysates, but the yields were sporadic and generally low in quantity.

In one embodiment, the present invention contemplates a method for detecting diabetogenic autoantigens comprising a NOD RIPTag mouse strain (commercially available as a cryopreserved embryo). In one embodiment, the NOD RIPTag mouse strain comprises a tumor, wherein said tumor comprises at least one diabetogenic autoantigen. In one embodiment, the method further comprises generating whole-cell membrane material from the tumor. In one embodiment, the method further comprises approximately 3-5 mg of whole-cell membrane material protein.

Another disadvantage regarding identification of autoreactive T cell autoantigens by conventional biochemical methods is trying to obtain tissue fractions in forms suitable for assaying directly with an autoreactive T cell clone. For example, many column fractions contain a detergent or high salt concentration that are toxic to T cells. In one embodiment, the present invention contemplates a method comprising a significant improvement in biochemical tissue fractionation procedures and autoreactive T cell analysis (see data described herein).

Another disadvantage regarding current attempts to identify autoreactive T cell autoantigens is related to the lack of technological improvements. In one embodiment, the present invention contemplates a method comprising identifying autoreactive T cell autoantigens by proteomic analysis. Recent literature has shown proteomics as a useful tool for the discovery and identification of new protein targets. In one embodiment, the method further comprises identifying a protein by mass spectrometry. In other embodiments, the method further comprises technology selected from the group comprising high pressure liquid column chromatography, ion sources, tandem mass spectrometry, or protein identification software. Although it is not necessary to understand the mechanism of an invention, it is believed that data collected using state-of-the-art proteomics technology can be analyzed using bioinformatics.

In summary, some embodiments contemplated by the present invention comprise identifying at least one unknown major autoantigen within a β-cell secretory granule membrane. Other embodiments comprise identifying beta cell autoantigens that have been previously reported, and/or herein identified for the first time. Although it is not necessary to understand the mechanism of an invention, it is believed that that the targets of the above BDC clones are likely to be relatively minor components (<1%) of the total granule membrane protein population, but will be routinely detectable using recent advances in proteomics technology and bioinformatics data analysis.

### V. Autoantigenic Chromogranins And Type 1 Diabetes

Chromogranin A has been suggested as a biomarker for pancreatic endocrine tumors. Gibril et al., “Zollinger-Ellison syndrome revisited: diagnosis, biologic markers, associated inherited disorders, and acid hypersecretion” Curr Gastroenterol Rep. 6:454-463 (2004). While a progressive development of pancreatic cancer may ultimately result in the development of diabetes, there is no suggestion that a chromogranin A autoantigenic sequence induces autoreactive T cells in pancreatic cancers.

Autoantigens inducing the development of diabetes via autoreactive T lymphocyte cells have been reported. Gianani et al., “Initial Results of Screening of Nondiabetic Organ Donors for Expression of Islet Autoantibodies” J Clin Endocrinol Metab 911855-1861 (2006). It was suggested that autoantigens identified as glutamic acid decarboxylase (GAD)65, insulin, and ICA512 (IA-2) may be involved in the initiation and development of diabetes. While the presence of chromogranin A was identified in the pancreatic ductal epithelial lining of transplantation donor tissues, chromogranin A was not suggested to be an autoantigenic compound.

A recombinant insulinoma antigen presenting cell (APC) line expressing wild type pancreatic β cell proteins (i.e., for example, chromogranin A) and displaying a diabetogenic class II MHC I-A^{g7} molecule (designated NitCIITA) has been reported to be capable of inducing an autoreactive diabetogenic BDC T cell clone (presumably via the IA^{g7} MHC complex). Suri et al., “First Signature of Islet β-Cell-Derived Naturally Processed Peptides Selected by Diabetogenic Class II MHC Molecules” J. Immunol. 180:3849-3856 (2008). A number of the expressed wild type β cell proteins were found to spontaneously bind to the I-A^{g7} receptor and displayed some homology at a suggested P1-P9 primary anchor binding sequence. In particular, a chromogranin A peptide comprising amino acid residues 407-423 (RPSSREDSVEARSDFEE (SEQ ID NO: 47)) was identified as a homolog compatible with the suggested binding site to the I-A^{g7} receptor and was speculated to represent an autoantigen for autoreactive T cell clones. Relative binding affinities to IA^{g7} complex between these homologous peptides were consistent with single amino acid substitutions within this nine amino acid sequence. However, despite activation of three autoreactive T cell clones (2522-113N T cell clone, 2533-30 T cell clone, 2535-5 T cell clone) by several isolated β cell proteins (including chromogranin A), these activated clones were not diabetogenic. Further, no data was provided showing that any chromogranin A peptide induced activation of a diabetogenic BDC T cell clone, only an intact NitCIIAT APC.

The data presented herein identify chromogranin A (ChgA) as the source of the antigen for BDC-2.5 and two other clones, based on mass spectrometric analysis of biochemically purified antigenic fractions from an islet beta cell tumor and on the demonstration that the antigen is missing from the pancreatic islet cells from ChgA^{-/-} mice. Peptide antigen mimotopes for these T cells that are identified herein confirm a previously reported common motif in the predicted p5 to p9 portion of the peptides [WX(R/K)M(D/E) (SEQ ID NO: 48)]. Judkowski et al., “Identification of MHC class II-restricted peptide ligands, including a glutamic acid decarboxylase 65 sequence, that stimulate diabetogenic T cells from transgenic BDC2.5 nonobese diabetic mice” J Immunol 166:908-17 (2001); and Yoshida et al., “Evidence for shared recognition of a peptide ligand by a diverse panel of non-obese diabetic mice-derived, islet-specific, diabetogenic T cell clones” Int Immunol 14: 1439-1447 (2002). These data suggested that aa354-362 (EDKRWSRMD (SEQ ID NO: 46)) was a possible antigen epitope in ChgA. Surprisingly, peptides containing this sequence did not activate the T cells, but the clones were activated by an overlapping peptide, WE14 (aa 359-372, WSRMDQLAKELTAE (SEQ ID NO: 11)), a natural cleavage product of ChgA. Curry et al., “WE-14, a chromogranin a-derived neuropeptide” Ann N Y Acad Sci 971:311-6 (2002). This finding was quite unexpected, because despite the presence of the antigen motif, the stimulating WE14 peptide lacks the N-terminal amino acids that would occupy positions p1 to p4 of the IA^{g7} peptide-binding groove that are normally important for stable MHCII binding. Binding studies suggest that the nine C-terminal amino acids of WE14 make up for this loss by interacting with IA^{g7} at a site outside of the normal binding groove.

It should be noted that autoantigen peptides disclosed herein and the putative chromogranin A I-A^{g7} P1-P9 anchor binding sequence as disclosed by Suri et al. have identity with amino acid residues in different regions of wild type Mus musculus chromogranin A isoforms. For example, autoantigen peptides as disclosed herein may be respectively compared as follows: i) isoform CRA_a (Accession No. EDL18857.1): amino acid residues 361-369 versus 419-427; ii) isoform CRA_d (Accession No. EDL18860.1): amino acid residues 356-364 versus 414-422; iii) isoform CRA_c (Accession No. EDL18859.1): amino acid residues 277-285 versus 335-343; iv) isoform CRA_b (Accession No. EDL18858.1) amino acid residues 115-123 versus 173-181; v) unnamed isoform (Accession No. BAE25920.1) amino acid residues 205-213 versus 263-271; and vi) full length chromogranin A (Accession No. NP_{—}031719.1) amino acid residues 354-362 versus 412-420. This comparison strongly suggests that some autoantigens disclosed herein are not homologs of the above speculative P1-P9 anchor binding sequence. In each isoform, various autoantigenic sequences are separated by fifty (50) amino acid residues. Because Suri et al. also reports that T cell clones stimulated by P1-P9 homologs are not diabetogenic, it is doubtful if some of the above amino acid residues are autoreactive peptides capable of activating a panel of CD+ Th1 T cells as exemplified in Table 1.

A. BDC Panel Antigenicity

Data provided herein exemplify some method embodiments as contemplated by the present invention. For example, methods are described for fractionating and separating β-cell membrane proteins, determining β-cell membrane protein antigenicity using a panel of diabetogenic T cell clones (See, Table 1), and identifying the β-cell membrane proteins using techniques including, but not limited to, mass spectrometry, high pressure liquid chromatography, or gel electrophoresis. The results presented herein describe purification and identification of autoantigenic peptide fractions that activate diabetogenic autoreactive T cells.

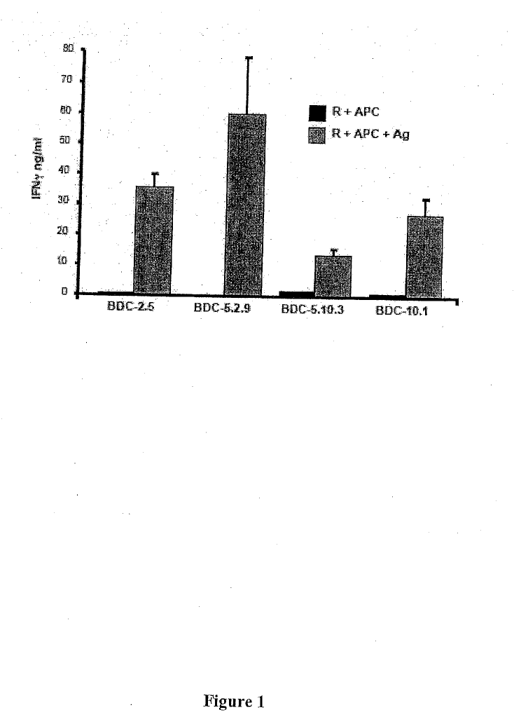

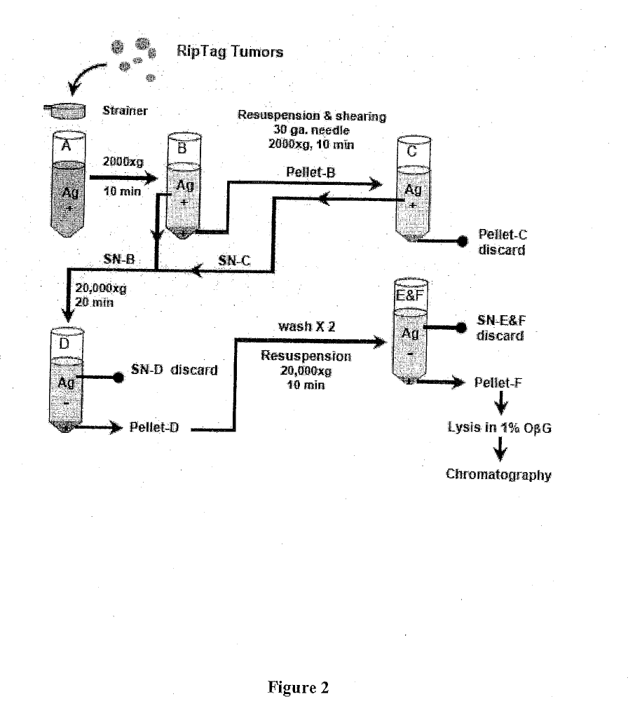

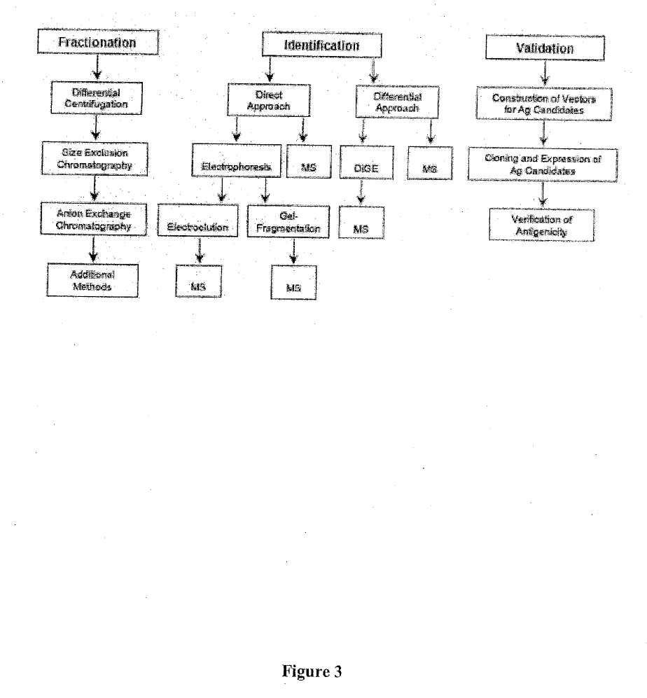

The data presented herein, show autoreactive responses of a BDC panel represented by four clones listed in Table 1 to a β-membrane autoantigens prepared in accordance with Example I, depicted as an ELISA for IFNγ. See, FIG. 1. The β-membranes are initially prepared using a 30 gauge strainer needle followed by centrifugation and washing steps. See, FIG. 2 in accordance with Example II. Further, an overall scheme for purification and identification of the autoantigens for the T cell clones is shown. See, FIG. 3. For example, beta cell membrane proteins may be fractionated by chromatography and identified through 1D and 2D SDS gel electrophoresis and mass spectrometry. Candidate antigens can then be cloned and expressed for verification of antigenicity with diabetogenic CD4 T cell clones.

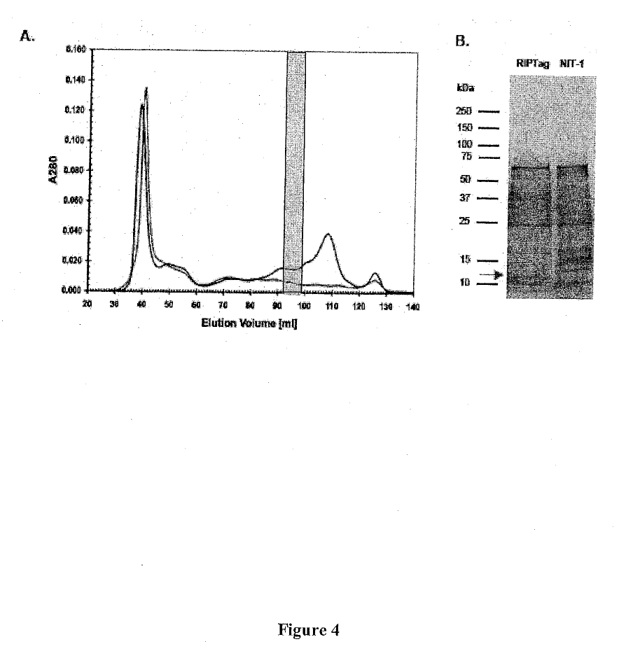

In one experiment, after differential centrifugation, the prepared membranes were then placed onto a size exclusion chromatography gel, wherein each fraction was tested for antigenic activity with the T cell clone BDC-2.5. See, FIG. 4. Antigens derived from RIPTag β-membrane fractions were detected in eluted fractions falling between approximately 90-100 ml elution volume. See, FIG. 5, gray region. Corresponding fractions of NIT-1 membranes were devoid of antigenic activity (i.e., for example, whole NIT-1 tumor cells). Antigenic activity for BDC-2.5 elutes within a small number of fractions from size exclusion chromatography (SEC) of a beta cell membrane lysate. SEC protein profiles from membrane preparations made from fresh RIP-Tag and the NIT-1 cell line are similar but not identical. Antigenicity was detected only in RIP-Tag membrane preparations. SDS PAGE analysis of the fractions in the antigenic zone indicates that there are some differences in proteins between freshly harvested beta tumor cells and NIT-1 cells in this region.

In another experiment, SDS-PAGE analysis was performed on antigenic fractions for the BDC-2.5 clone after combined SEC and IEX. See, FIG. 5. For example, combined antigenic fractions from SEC were applied to an anion exchange column and eluted with a NaCl gradient, yielding three fractions (elution volumes 21-23) containing antigenic activity for the clone. Fractions 21-23 were dialyzed, concentrated and applied to SDS-PAGE. A majority of the protein content and antigenicity of a T-cell clone BDC 2.5 preparation was found eluting in fraction 22. See, FIG. 5; shaded portion.

B. Identification of Chromogranin A (ChgA) as A BDC-2.5 Autoantigen

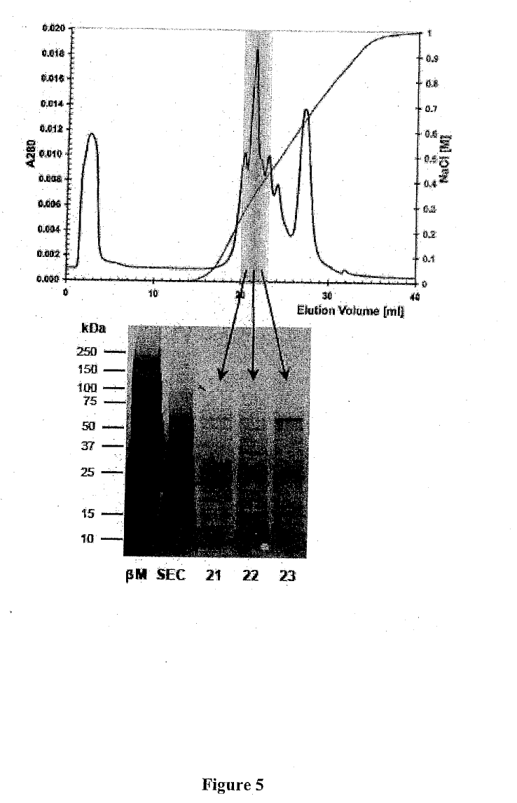

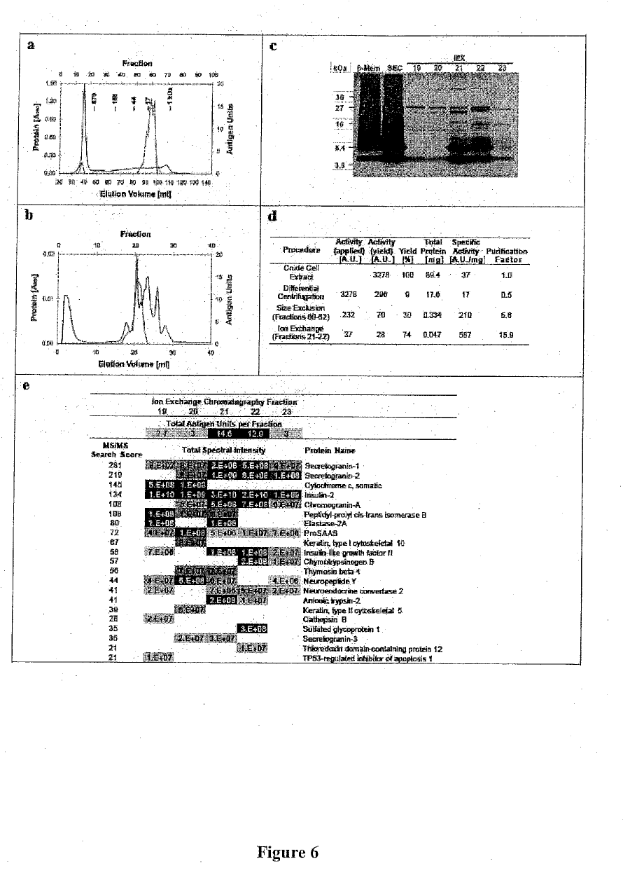

Identification of candidate antigens for the BDC clones has been reported by biochemical separation and proteomic analysis in a partially purified protein preparation from the secretory granules of a beta cell adenoma tumor. Hamaguchi et al., “NIT-1, a pancreatic beta-cell line established from a transgenic NOD/Lt mouse” Diabetes 40:842-849 (1991). Mass spectrometry was then used to identify autoantigens within the SEC/IEX RIP-Tag β-membrane fractions. For example, a mass spectrometric analysis of antigenic fractions obtained from SEC and IEX chromatography. See, FIG. 6A and FIG. 6B. The antigen was tracked during isolation by observing stimulation of the prototypic T cell clone, BDC-2.5. The final highly purified fractions also stimulated two other T cell clones, BDC-10.1 and BDC-5.10.3 (data not shown). A representative silver-stained SDS-PAGE gel illustrating the protein content of the combined antigen-containing SE fractions (lane 2) and peak antigenic fractions from IEX (lanes 3-7). See, FIG. 6C. The relative degree of purification obtained with each step of separation is also summarized. See, FIG. 6D.

To identify the proteins present in the IEX fractions containing the antigenic activity, tryptic digests from each fraction were analyzed by mass spectrometry. Resulting peptides were sequenced and matched to proteins via a search of the Swissprot protein database using the database search program Spectrum Mill (Agilent Technologies). A total of 21 proteins were identified in fractions 19-23 using this technique and spectral intensities indicate relative abundance of individual proteins identified in each fraction. See, FIG. 6E. The mean intensity of the spectrum for each peptide is color-coded, with the darker colors indicating a higher intensity (e.g., red indicates a higher intensity than yellow). A comparison of spectral intensities with corresponding antigenicity in each fraction resulted in a list of potential antigen candidates including secretogranins 1 and 2, insulin-like growth factor II, and ChgA. Nearly identical results were obtained with repeated experiments, with the exception of insulin-like growth factor which was only identified in one experiment.

The data presented herein shows this technique can unambiguously identify the presence of a particular protein within a fragmented preparation. Proteins in each fraction were digested and analyzed using ion trap mass spectrometry and data was searched using the database searching program Spectrum Mill®. This information can be used semi-quantitatively to determine in which fraction the majority of the protein is present.

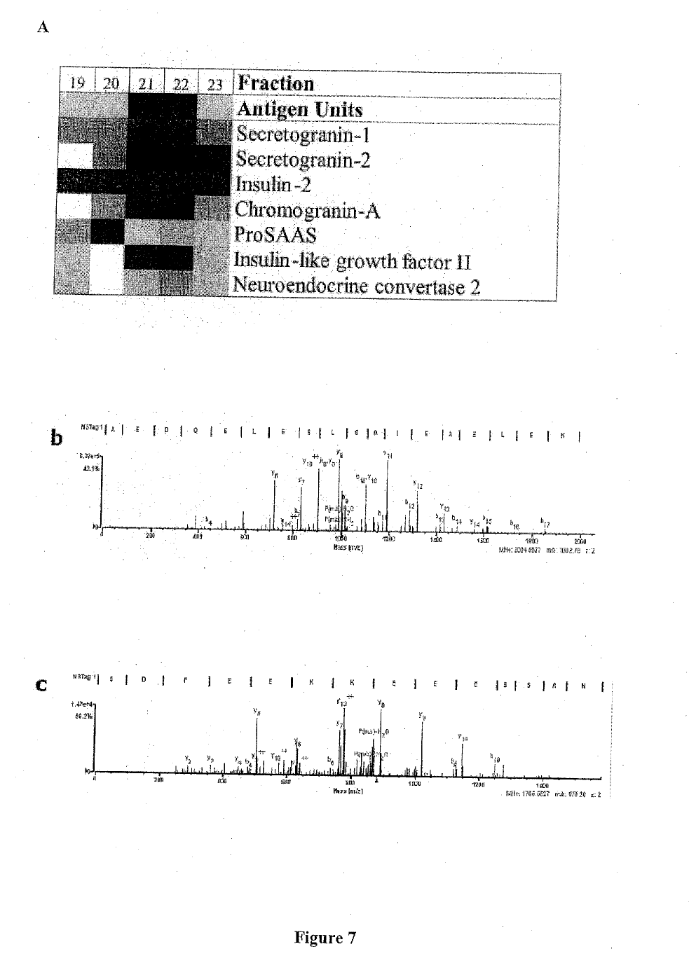

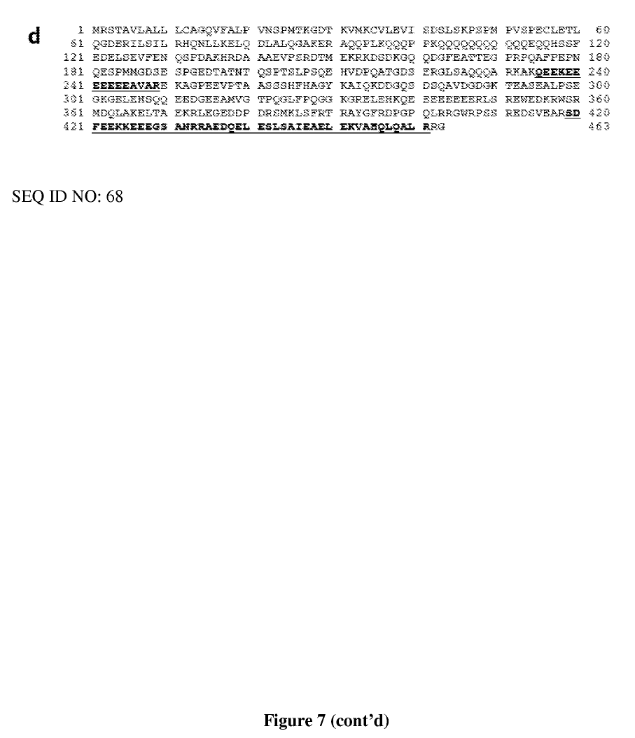

The best protein candidates were then selected from this analysis. See, FIG. 7A. For example, proteins in highly purified antigenic IEX fraction (i.e., for example, fraction 21) and adjacent fractions that displayed lower antigenic activity (i.e., for example, fractions 19, 20, 22 and 23) were digested with trypsin and after separation by HPLC, were analyzed using an ion trap mass spectrometer. Resulting spectra were searched against a protein sequence database. Of particular interest are members of the secretogranin family of proteins, secretogranins 1 and 2 and chromogranin A as their relative abundance matches up with the amount of antigen in the antigenic fractions 19-23, with fractions 21 and 22 containing the most antigen. Insulin is in high abundance in all fractions and therefore is not a good match with the antigenicity of the chromatographic fractions.

Representative mass spectra and matching sequence are shown for two of the selected peptides. See, FIG. 7B and FIG. 7C. Overall, six (6) ChgA peptides mapping to the C-terminal portion of the protein (i.e., for example, aa 233-463) were confidently identified in highly antigenic fractions with four (4) peptides being reproducibly detected in 3 experiments. See, FIG. 7D. The predicted molecular weight of this potentially truncated ChgA protein (i.e., for example, aa 233-463) is approximately 26 kDa, which is consistent with results from SEC. Based on these results, and the fact that the distribution of ChgA in the fractions correlated well with antigenicity, ChgA was identified as a candidate antigen.

In summary, the above data demonstrate that the disclosed improved technology can be used to identify specific proteins within any fragmented protein preparation. These include, but are not limited to, assay of β-cell antigens with a panel of diabetogenic CD4 T-cell clones, extraction of autoantigen from β-cell membranes of RIPTag tumors, antigen enrichment methods yielding fractions that can be assayed with the T cell clones for antigenicity, or protein identification using mass spectrometry.

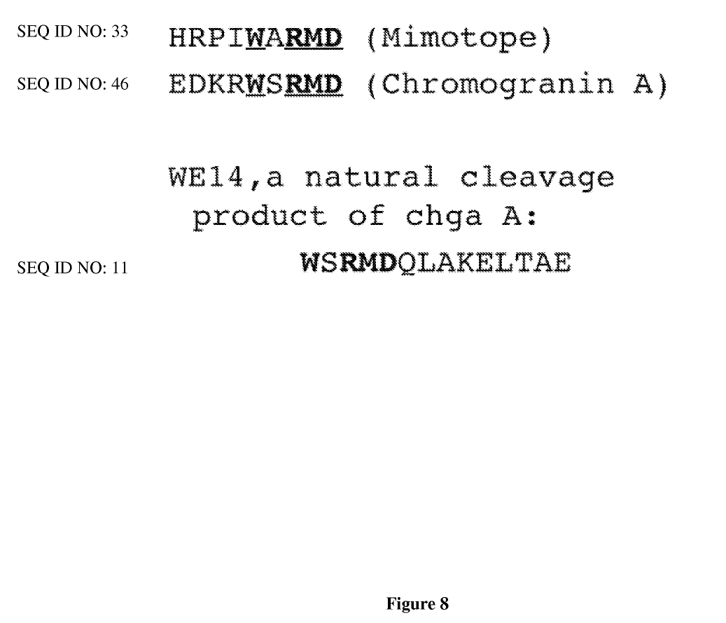

Of the protein candidates identified by mass spectrometry, chromogranin A was the best candidate because it contained a peptide, EDKRWSRMD (SEQ ID NO: 46), which was predicted to bind well to the NOD IA^{g7} MHCII molecule and had homology to the related peptide mimotopes, HRPIWARMD (SEQ ID NO: 33) and HIPIWARMD (SEQ ID NO: 36) (Yoshida et al 2002), that activate two of the T cell clones used in the study, BDC-2.5 and BDC-10.1, respectively. See, FIG. 8.

A second line of inquiry was based on screening a baculovirus based IA^{g7}-peptide display library for peptides that could activate the BDC-2.5 and BDC-10.1 clones as well as a third T cell clone BDC-5.10.3. See, Table 2.

TABLE 2
Chromogranin A-Like Fragment Stimulation Of
INFγ-Production.

| | Peptide | BDC- | BDC- | BDC- |
|---|---|---|---|---|
| Protein Source | Sequence | 2.5 | 10.1 | 5.1 |
| BV Library | RLGLWVRME | +++ | +++ | +++ |
| | (SEQ ID NO: 37) | | | |
| GDP-mannose | RVGQWARME | + | +++ | − |
| pyrophosphate B | (SEQ ID NO: 38) | | | |
| DNAjc14 | RLGGWARMM | ++ | − | + |
| | (SEQ ID NO: 39) | | | |
| Carboxypeptidase | ELMEWWKMM | − | − | − |
| E | (SEQ ID NO: 40) | | | |
| Kirrel-2 | PRITWTRMG | − | − | − |
| | (SEQ ID NO: 41) | | | |
| Chromogranin A | EDKRWSRMD | − | − | − |
| | (SEQ ID NO: 46) | | | |

A single peptide emerged that strongly stimulated all three clones. Its sequence, RLGLWVRME (SEQ ID NO: 37), was also related to previous mimotopes. Based on this sequence multiple strategies were used to search genomic sequence databases and related peptides were found in a number of proteins including the chromogranin A peptide, EDKRWSRMD (SEQ ID NO: 46).

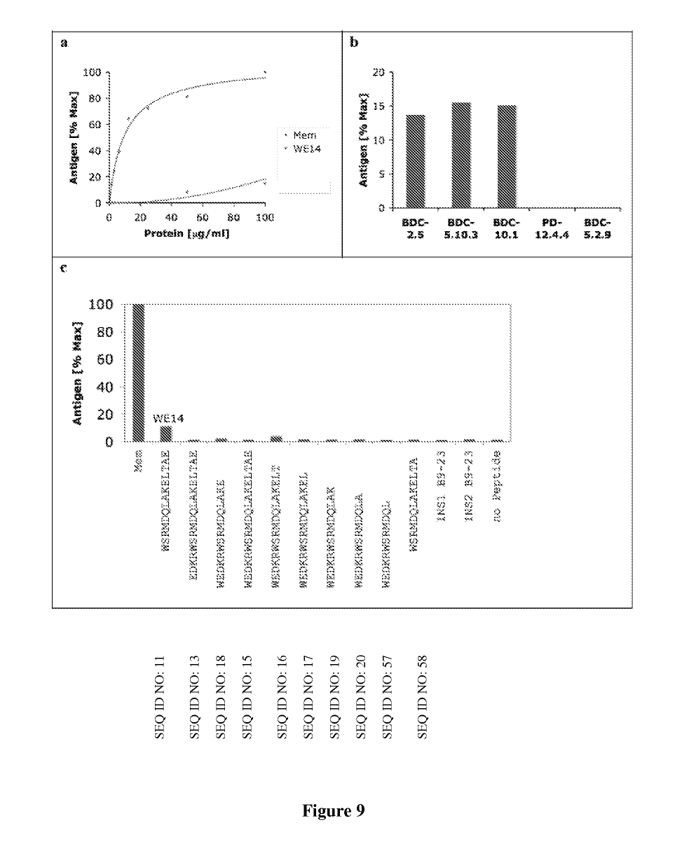

Since chromogranin A was the only protein identified by both of these approaches, the IA^{g7} binding EDKRWSRMD (SEQ ID NO: 46) peptide was tested for its ability to activate the clones. Surprisingly this peptide did not stimulate any of the clones. However, a naturally processed peptide of chromogranin A, WE-14 (WSRMDQLAKELTEA (SEQ ID NO: 49)) did stimulate the clones and contains only the last five amino acids of the predicted IA^{g7} binding peptide, and therefore is predicted to only partially fill the IA^{g7} peptide binding groove. Nevertheless, when tested this peptide stimulated all three T cell clones. See, FIG. 9. However, WE14 only weakly stimulated clone BDC-2.5. While concentrations of 100 mg/ml β-membrane lead to a maximal T cell response (100%), the same concentrations of WE14 peptide only lead to a response of ˜15%. The T cell clones BDC-2.5, BDC-5.10.3 and BDC-10.1 yield a comparable response to 100 mg/ml WE14 peptide. The data herein also shows results of testing a series of peptides related to WE14; these yielded only background level responses at a concentration of 100 mg/ml peptide. See, FIG. 9.

As the WE-14 peptide was much less potent in activating the T cell clones when compared to the active fraction purified from the beta cell tumor, the possibility is raised that some other form of the peptide, perhaps requiring some type of post-translational modification, is the natural antigen for these clones.

A post translational modification outside the predicted MHC binding site at the amino acid Glutamine and/or Lysine may turn the peptides into strong antigens. This modification includes, but is not limited to, the addition of a functional group to the amino acid glutamine and/or lysine. The functional group may include, but is not limited to, the formation of a reactive species (such as an anhydride) at the epsilon functional group of the amino acid glutamine.

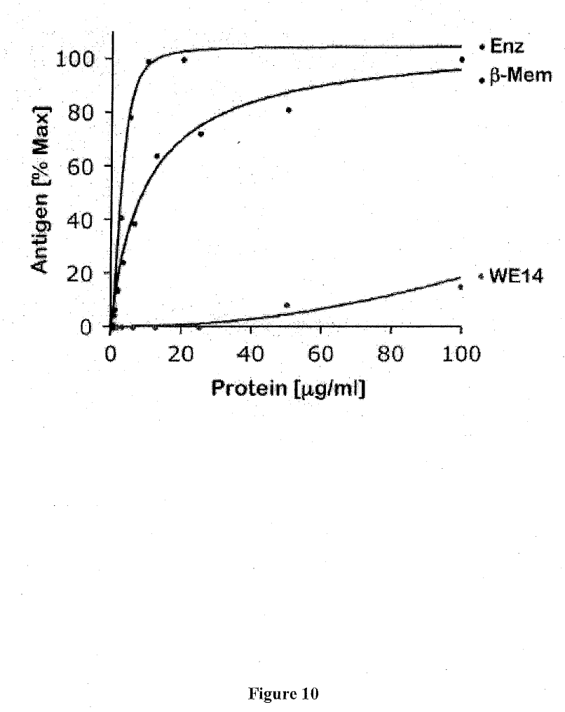

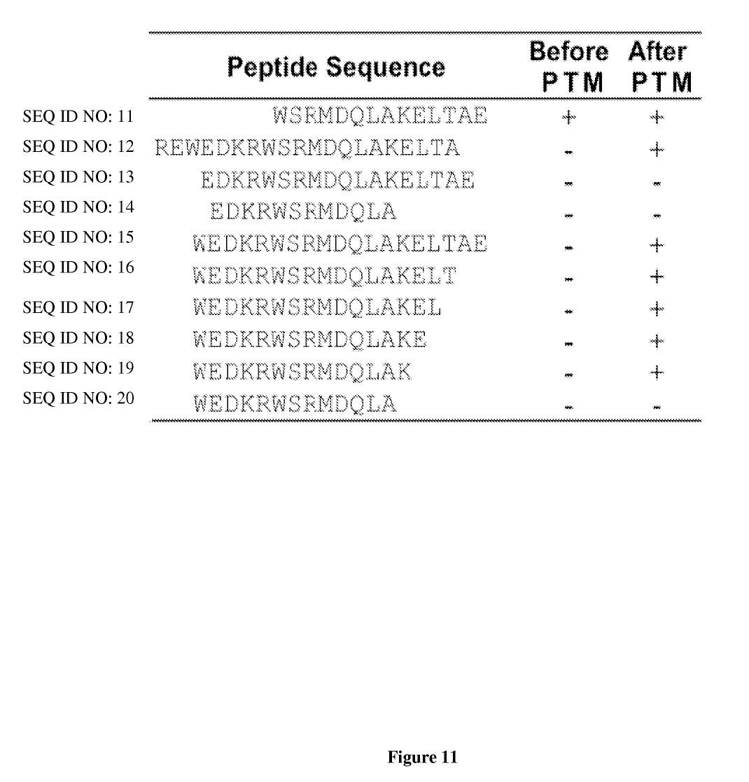

FIG. 10 indicates that post-translational modifications of WE14 with the enzyme transglutaminase does render this peptide highly antigenic. See, FIG. 10. Other peptides may also become antigenic upon enzymatic conversion. See, FIG. 11.

C. ChgA Stimulation of T Cell Clones

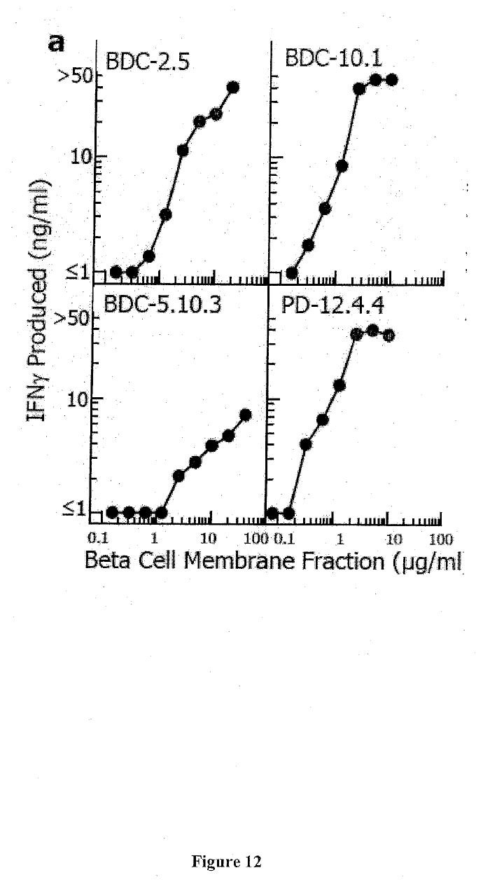

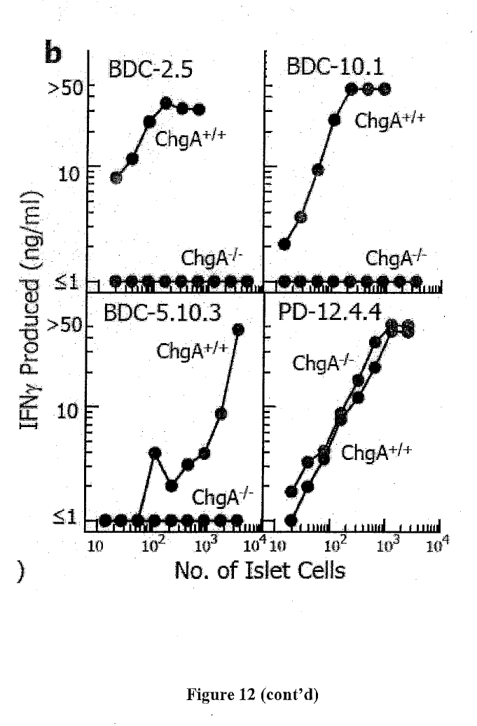

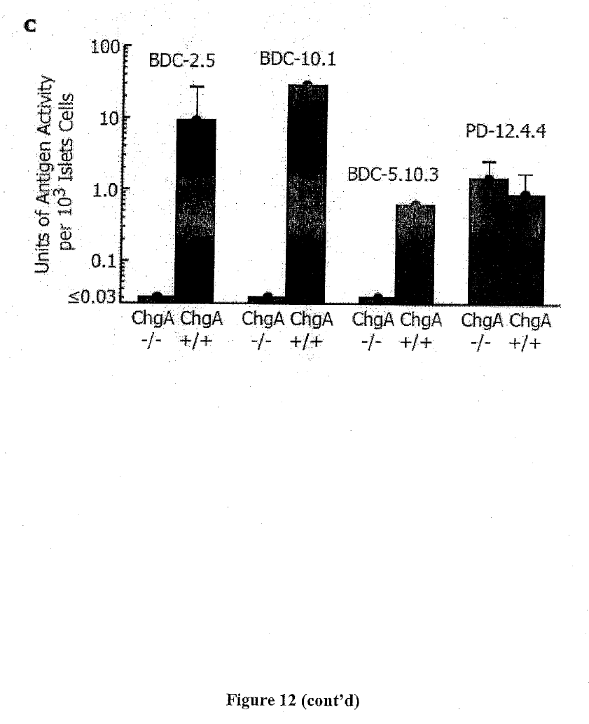

The data presented herein determines ChgA as a source of antigen for the BDC-2.5 T cell clone, BDC-10.1 clone, and BDC-5.10.3 clone, by comparing the levels of antigen in pancreatic islet cells from ChgA^{-/-} vs. ChgA^{+/+} mice. Mahapatra et al., “Hypertension from targeted ablation of chromogranin A can be rescued by the human ortholog” J Clin Invest 115:1942-52 (2005). While the ChgA^{-/-} mice are apparently healthy and normal in most respects, they do exhibit some irregularities in terms of islet numbers, size, and insulin secretion. Portela-Gomes et al., “The importance of chromogranin A in the development and function of endocrine pancreas” Regul Pept 151:19-25 (2008). Therefore, a PD-12.4.4 insulin reactive clone was included as a control for any global deficiencies in the ChgA^{-/-} mice in islet beta cell or granule formation. Daniel et al., “Epitope specificity, cytokine production profile and diabetogenic activity of insulin-specific T cell clones isolated from NOD mice” Eur J Immunol 25:1056-1062 (1995). The T cell clones were cultured with IA^{g7} antigen-presenting cells and various numbers of islet cells from the ChgA^{-/-} vs ChgA^{+/+} mice as a source of antigen and the beta cell tumor antigen preparation was used as a positive control. All four T cell clones (BDC-2.5, BDC-10.1, BDC-5.10.3, and PD12.4.4) activated IFNγ production in beta cell membranes. See, FIG. 12A. Further, the PD-12.4.4 insulin reactive clone responded equally well to islet cells from either ChgA or ChgA^{-/-} mice, suggesting equivalent insulin levels in individual islet beta cells from either source. The BDC-2.5, BDC-10.1, BDC-5.10.3 clones also responded well to ChgA^{+/+} islet cells, but not at all to any number of islet cells tested from ChgA^{-/-} mice. See, FIG. 12B and FIG. 12C. These data confirm that ChgA as a source of an antigen for these T cells.

D. Peptide Mimotopes From Diabetogenic Clones

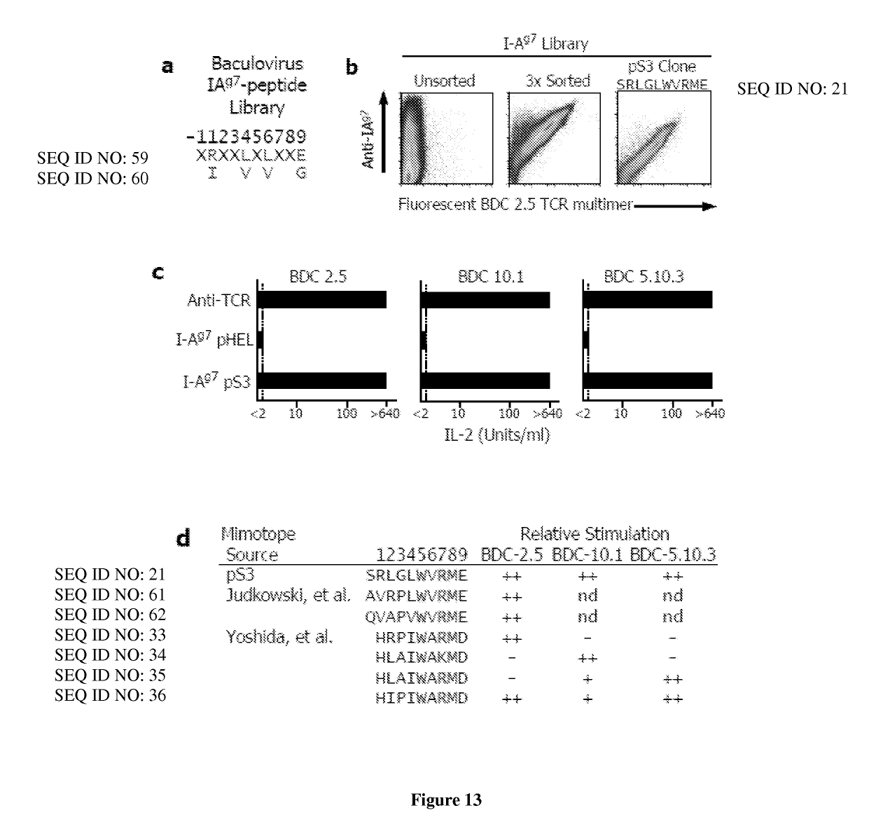

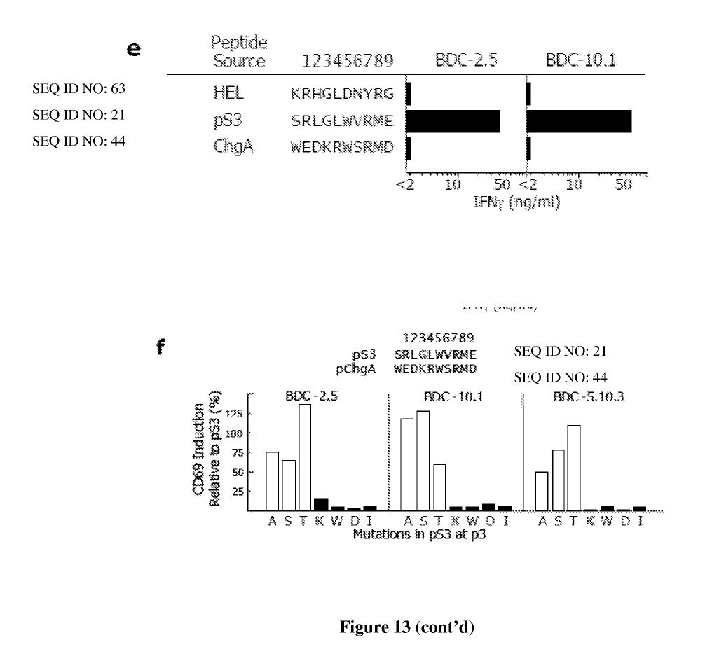

Various types of peptide libraries can be screened to identify peptide mimotopes for one or more of the BDC T cell clones. See, FIG. 13; Judkowski et al., “Identification of MHC class II-restricted peptide ligands, including a glutamic acid decarboxylase 65 sequence, that stimulate diabetogenic T cells from transgenic BDC2.5 nonobese diabetic mice” J Immunol 166:908-917 (2001); and Yoshida et al., “Evidence for shared recognition of a peptide ligand by a diverse panel of non-obese diabetic mice-derived, islet-specific, diabetogenic T cell clones” Int Immunol 14:1439-1447 (2002). These libraries may be constructed to contain peptides that would bind well to IA^{g7} by placing suitable anchor residues at various positions of the peptide (i.e., for example, p1, p4, p6 and p9). Amino acids at other peptide positions were randomized. All of these studies identified mimotopes with similar sequences from p5 to p9-WX(R/K)M(E/D) (SEQ ID NO: 50), but the sequences varied greatly from p1 to p4.

A peptide mimotope was reported for three of the BDC clones from a library of peptides that covalently bound to IA^{g7} and displayed on the surface of insect cells via baculovirus. Crawford et al., “Mimotopes for alloreactive and conventional T cells in a peptide-MHC display library” PloS Biol 2:E90 (2004); and Crawford et al., “Use of baculovirus MHC/peptide display libraries to characterize T-cell receptor ligands” Immunol Rev 210:156-170 (2006). In the baculovirus encoded library (˜10⁷ independent clones), the peptide amino acids at the four major IA^{g7}-binding positions were minimally varied: p1-Arg or Leu, p4-Leu or Val, p6-Leu or Val and p9-Gly or Glu.

The amino acids at p1, p2, p3, p5, p7 and p8 were fully randomized to all 20 amino acids. See, FIG. 13A. Insect cells were infected with the library at a multiplicity of infection of <1 such that most infected cells expressed a single member of the library. The few infected cells expressing an IA^{g7}-peptide combination capable of binding a fluorescent, soluble, multimeric version of BDC-2.5 TCR were isolated using flow cytometry. See, FIG. 13B, panel 1). These cells were used to create a new enriched viral stock. This experimental cycle was performed twice more, producing a highly enriched population of viruses that expressed IA^{g7}-peptide combinations, most of which bound the BDC-2.5 TCR. See FIG. 13B, panel 2). Cloned viruses from this enriched population were retested for BDC-2.5 TCR binding. The viral DNA was sequenced for all TCR-binding clones and encoded a single peptide sequence, RLGLWVRME (SEQ ID NO: 37), denoted as pS3. See, FIG. 13B, panel 3).

T cell hybridomas bearing TCR from either the BCD-2.5, BDC-10.1 or BDC-5.10.3 T cell clones were tested for their ability to recognize the covalent IA^{g7}-p53 complex using B7/ICAM-expressing insect cells as artificial APCs. See, FIG. 13C; Crawford et al., “Mimotopes for alloreactive and conventional T cells in a peptide-MHC display library” PloS Biol 2:E90 (2004); and Crawford et al., “Use of baculovirus MHC/peptide display libraries to characterize T-cell receptor ligands” Immunol Rev 210: 156-170 (2006). Three hybridomas were maximally activated by the pS3 mimotope, providing support for the hypothesis that these three T cells were reactive to the same self-antigen and that the pS3 sequence might resemble that of the natural antigen.

Previous reports described a technique of positional scanning peptide libraries to identify antigen mimotopes for one or more of these three BDC T cell clones. Searches of databases with these mimotope sequences had failed to turn up the natural source of the antigen. Judkowski et al., “Identification of MHC class II-restricted peptide ligands, including a glutamic acid decarboxylase 65 sequence, that stimulate diabetogenic T cells from transgenic BDC2.5 nonobese diabetic mice” J Immunol 166:908-917 (2001); Yoshida et al., “Evidence for shared recognition of a peptide ligand by a diverse panel of non-obese diabetic mice-derived, islet-specific, diabetogenic T cell clones” Int Immunol 14:1439-1447 (2002). In the present data, one striking feature found within several mimotopes, and pS3, was a common WX(R/K)M(D/E) (SEQ ID NO: 48) motif in amino acids p5-p9 of the peptides. See, FIG. 13D. An examination of a ChgA sequence identified this motif to reside within a C-terminal portion thereby suggesting a possible core peptide epitope (i.e., for example, p-1 to p9) within ChgA comprises aa353-362 (WEDKRWSRMD (SEQ ID NO: 44)). This possible ChgA core peptide epitope ChgA sequence was then incorporated into a baculovirus IA^{g7} construct (e.g., IA^{g7}-pChgA) and the resulting virus was used to infect B7/ICAM-expressing insect cells. Cells infected with IA^{g7}-pHEL and IA^{g7}-p53 were used as negative and positive controls, respectively.

These infected cells were tested for activation of the BDC-2.5 and BDC-10.1 T cell clones. As expected, the IA^{g7}-pHEL expressing cells did not activate either clone and the IA^{g7}-p53 cells strongly activated both clones. Unexpectedly, the IA^{g7}-pChgA cells failed to stimulate either T cell. See, FIG. 13E. This result was particularly surprising, since IA^{g7}-pChgA was the only sequence with any homology to the antigen mimotopes. However, a close comparison of the IA^{g7}-pChgA sequence with the mimotope sequences suggested a possible reason for the failure. Although it is not necessary to understand the mechanism of an invention, it is believed that while the N-terminal sequences of the mimotopes (p-1 to p4) vary considerably, they all have a small noncharged amino acid at p3 (Gly, Ala, or Pro). It is further believed that, the IA^{g7}-ChgA peptide has a large, positively charged amino acid (Lys) at the p3 position. See, FIG. 13D. It is further believed that this Lys could be providing steric hindrance of antigen recognition by T cells. A mutational study of this position in the pS3 mimotope testing variants of pS3 with the Gly at p3 mutated to many other amino acids further strengthens this explanation. See, FIG. 13F. Apparently, substitutions with amino acids with small side chains (Ala, Ser, or Thr) preserved the ability of the mimotope to stimulate all three BDC T cell hybridomas. However, changing this amino acid to Lys, the p3 amino acid of the ChgA peptide, or to several other amino acids with large side chains, eliminated the activation of all three T cells.

E. A Natural ChgA Antigenic Epitope

The data presented here suggest that an N-terminal part of the ChgA peptide interferes with T cell recognition, mediated by a naturally processed ChgA-derived peptide, WE14 (WSRMDQLAKELTAE (SEQ ID NO: 11)). Curry et al., “WE-14, a chromogranin a-derived neuropeptide” Ann N Y Acad Sci 971:311-6 (2002). WE-14 lacks the first five (5) N terminal amino acids (p-1 to p4) of the IA^{g7}-ChgA peptide tested above (i.e., for example, WEDKR (SEQ ID NO: 51)), but still has the common mimotope motif and at least a portion of the C-terminal end of the peptide. See FIG. 14A. Although it is not necessary to understand the mechanism of an invention, it is believed that this peptide would bind poorly to IA^{g7} because placement of the WSRMD (SEQ ID NO: 52) portion of the peptide in the p5 to p9 position would only partially fill the peptide binding groove, eliminating many of the usually conserved interactions between MHC and peptide involving p-1 to p4.

A soluble synthetic version of WE14 was therefore tested for its ability to activate the three T cell clones, comparing it to the pS3 mimotope and the control beta cell tumor antigen preparation. See, FIG. 14B. The very potent pS3 mimotope stimulated all three BDC clones maximally at all concentrations tested. All three clones also responded to the beta cell antigen preparation. WE14 peptide also stimulated all three BDC clones, confirming that the elimination of the portion of ChgA that would be expected to fill the p-1 to p4 part of the IA^{g7}-binding groove may mediate T cell recognition. The insulin-reactive PD-12.4.4 T cell clone comprising an insulin-derived peptide B:9-215 epitope was used as a negative control. As expected, the PD-12.4.4 clone responded to an insulin peptide and/or beta cell antigen preparation, but not to pS3 or either of the ChgA-derived peptides. It is worth noting that the synthetic WE14 peptide was also considerably less potent than the beta cell antigen preparation, suggesting that the natural version of this peptide may be subject in vivo to some alternate form of processing or to post-translational modification.

In order to confirm the unique binding register of the WE14 peptide, a series of peptides comprising N-terminal extensions and/or C-terminal deletions of WE14 were evaluated for their ability to stimulate the BDC-2.5 T cell clone (FIG. 6a) or to bind to IA^{g7}. See, FIG. 15A. These data were compared to inhibition of IA^{g7} binding by a biotinylated control peptide (i.e., for example, pHEL). See, FIG. 15B. Further, a pS3 mimotope was used as the positive control peptide and an irrelevant peptide from moth cytochrome c, pMCC, was the negative control peptide. The data were analyzed to determine the stimulatory or binding capacity of the peptides relative to WE14. See, FIG. 15C.

Sequential truncation of WE14 (i.e., for example, WL11, WA8, WD5) from the C-terminus resulted in decreasing stimulatory activity. Compare, FIG. 15A and FIG. 15C. The shortest peptide (WD5) (that contained only the WX(R/K)M(E/D) (SEQ ID NO: 50) motif, lacked any activity at all. The detrimental effect of these truncations was even more dramatic in the IA^{g7} binding assay. The pS3 mimotope appeared to have a higher affinity for IA^{g7} as compared to WE14. Further, a truncation of as few as 3 C-terminal amino acids from WE14 (i.e., for example, WL11) reduced IA^{g7} binding to a level indistinguishable from the negative control peptide. Compare, FIG. 15B and FIG. 15C. These data indicate that the C-terminal 9 amino acids of WE14 participate in optimal binding and stimulation by the peptide.

The data also showed that extending WE14 by 4 amino acids (i.e., for example, EDKR (SEQ ID NO: 53), denoted EE18) had no effect on IA^{g7} binding. Compare, FIG. 15B and FIG. 15C. However, the BDC-2.5 stimulatory response was virtually eliminated. Compare FIG. 15A and FIG. 15C. Although it is not necessary to understand the mechanism of an invention, it is believed that these observations suggest that these added amino acids may be incompatible with T cell recognition Likewise, extending an WD5 peptide with the EDKR (SEQ ID NO: 53) sequence (i.e., for example, ED9) also produced a peptide that failed to stimulate BDC-2.5. Compare FIG. 15A and FIG. 15C. Surprisingly, however, the ED9 peptide, despite its length, did not bind to IA^{g7}. Compare FIG. 15B and FIG. 15C. Similar results were seen with extensions of WD5 by 1 amino acid (i.e., for example, RD6), 2 amino acids (i.e., for example, KD7) or 3 amino acids (i.e., for example, DD8) (data not shown).

Although it is not necessary to understand the mechanism of an invention, it is believed that that because EE18 binds to IA^{g7}, as well as WE14, the ED9 EDKR (SEQ ID NO: 53) extension is unable to fill the p1-p4 portion of IA^{g7} binding groove properly, most likely because of a problem with the p4R anchor position. It is further believed that WE14 employs an unusual means of binding to IA^{g7} via interaction of its C-terminal 9 amino acids with a site outside of the normal IA^{g7} binding groove.

While there are other examples of peptide amino acids flanking the binding groove contributing to MHCII binding and T cell recognition, optimization using a long stretch of flanking amino acids as shown herein is unprecedented. Carson et al., “T cell receptor recognition of MHC class II-bound peptide flanking residues enhances immunogenicity and results in altered TCR V region usage” Immunity 7:387-99 (1997); Arnold et al., “The majority of immunogenic epitopes generate CD4+ T cells that are dependent on MHC class II-bound peptide-flanking residues” J Immunol 169:739-749 (2002); Levisetti et al., “The insulin-specific T cells of nonobese diabetic mice recognize a weak MHC-binding segment in more than one form” J Immunol 178, 6051-6057 (2007).

F. Functional ChgA Antigens

In one embodiment, the present invention contemplates a method comprising generating a plurality of functional ChgA antigens, wherein amino acids are removed or altered thereby avoiding interference with T cell receptor (TCR) binding to the peptide-IA^{g7} complex. In one embodiment, N-terminal amino acids are removed or altered, wherein TCR affinity is modulated. Although it is not necessary to understand the mechanism of an invention, it is believed that depending on a particular TCR, various ChgA-derived peptide sequences, but not a full length ChgA protein, can avoiding binding interferences with the TCR IA^{g7} groove. In one embodiment, the amino acid removal or alterations are p1 or p4 amino acid removals or alterations. Although it is not necessary to understand the mechanism of an invention, it is believed that p1 and p4 amino acids result in peptide optimization that promote strong IA^{g7} binding, thereby making C-terminal extensions (i.e., for example, WE14) less preferred. For example, the various library strategies reported herein did not produce mimotopes that readily suggested WE14 as the source of a ChgA antigen.

The data presented herein show that the synthetic WE14 peptide is at least 1000 fold less potent than the pS3 mimotope in activating the BDC clones. However, WE14 also is considerably less potent than the antigen preparation from the beta cell adenoma tumor, despite the fact that ChgA is only one of a number of proteins in this fraction. Although it is not necessary to understand the mechanism of an invention, it is believed that the naturally processed antigen may differ from the synthetic WE14 peptide in some way, for example, due to some form of post-translational modification that improves either peptide binding to IA^{g7} or TCR binding to the complex.

Previous studies have detected pancreatic WE14. Curry et al., “Colocalization of WE-14 immunostaining with the classical islet hormones in the porcine pancreas” Adv Exp Med Biol 426:139-144 (1997). However, WE14 has not been detected in purified antigenic fractions from pancreatic beta tumor cells. Rather, since the data presented herein indicates that it is the C-terminal portion of ChgA that encodes WE14, post-translational processing and/or modification in antigen-presenting cells may be required to generate an active WE14 epitope.

Post-translational modification of antigens has received considerable attention in T cell mediated inflammatory disease studies. For example, in rheumatoid arthritis, citrullination of arginines by peptidylarginine deiminases has been discussed as a possible mechanism for improving binding of self-peptides to DR4 by creating an improved p4 anchor residue. Hill et al., “Cutting edge: the conversion of arginine to citrulline allows for a high-affinity peptide interaction with the rheumatoid arthritis-associated HLA-DRB1*0401 MHC class II molecule” J Immunol 171:538-41 (2003). Also, in celiac disease, tissue trans-glutaminase conversion of glutamine to glutamic acid, in particular gluten peptides, creates new T cell epitopes. Reports suggest that this process improves peptide binding to the relevant HLA-DQ alleles through changing anchor residues. Tollefsen et al., “HLA-DQ2 and -DQ8 signatures of gluten T cell epitopes in celiac disease” J Clin Invest 116:2226-2236 (2006); and Hovhannisyan et al., “The role of HLA-DQ8 beta 57 polymorphism in the antigluten T-cell response in coeliac disease” Nature 456:534-538 (2008). Although it is not necessary to understand the mechanism of an invention, it is believed that both of these enzymes are induced locally by inflammation wherein enhanced antigen presentation can be induced locally in target tissues, but not in the thymus, allowing potentially pathogenic T cells to escape thymic deletion. Similarly, the WE14 peptide has potential amino acids for both of these post-translational modifications, as well as others, such as lysine hydroxylation.

### VI. Improved Protein Isolation And Purification Technology

As indicated above, severe limitations related to the isolation and purification of protein autoantigens have been the cause of slowly developing therapeutics for autoimmune diseases. The data described above was performed using an experimental design incorporating significant improvements of several laboratory techniques. See, FIG. 6. In brief, there are three experimental phases involved in identifying autoreactive T cell autoantigens; i) chromatographic separation of cellular fractions; ii) identification of protein candidates using mass spectrometry; and iii) validation through expression and testing of candidate antigens. Details of each part of this plan are provided below under the specific aims.

A. Chromatographic Separation

In one embodiment, the present invention contemplates a method comprising identifying antigens using a chromatographic separation procedure and mass spectrometry. In one embodiment, the antigen comprises a β-cell antigen. In one embodiment, the β-cell antigen activates a panel of diabetogenic CD4 T cell clones. In one embodiment, the method further comprises determining whether the CD4 T cell clones are reactive with epitopes in a single protein or in a group of proteins.

Previous studies have indicated that several diabetogenic T cell clones (see Table 1), react with antigens enriched in the membranes of insulin secretory granules. See, FIG. 1. However, highly purified fractions of antigenic membrane material obtained through chromatography are shown herein to result in unambiguous identification of the antigens. For example, the initial fractionation steps may include, but are not limited to, combining size exclusion and ion exchange chromatographic separations to produce a small number of T cell clone antigenic fractions. These fractions, however, still contain a fair number (40-50) of bands on silver-stained SDS gels (i.e., not yet sufficiently purified to obtain unambiguous identification). Nonetheless, a SEC/IEX combination optimizes protein purification in preparation for a subsequent mass spectrometry analysis.

Another improvement further decreases the number of potential antigen candidates. Molecular weight cut-off (MWCO) membranes (e.g., Microcon YM Centrifugal Filter Units, Millipore) can be used by which small proteins can be separated from large proteins (e.g., 30 kDa cut-off) in a centrifugation step. The SDS-PAGE presented herein indicate that most of the potential antigenic proteins have a molecular weight of <70 kDa. See, FIG. 4. Therefore, by using a membrane cut-off below 70 kDa (e.g., 30 kDa), this fractionation step is quickly and easily improved. Further, while MWCO filters result in low recovery due to an inherent “stickiness” of the filter, rigorous pupating of the sample improves the yield.

One reason for using a low molecular weight cut-off step is to eliminate insulin and pro-insulin from the mixture of proteins because these proteins are major components of the secretory granule. Although it is not necessary to understand the mechanism of an invention, it is believed that in order to discover unknown autoantigens in T1D, removal of low molecular weight proteins including any free forms (i.e., not aggregated) of insulin/proinsulin from the membrane fractions will improve assay sensitivity by removing competing and contaminating proteins. In addition to reducing or eliminating the presence of insulin in the assay preparations, a control insulin-specific clone, PD 12-4.4 is used to detect any insulin-based antigenic contamination. (25). For example, the 12-4.4 T cell clone is believed to be insulin B9-23 peptide-specific but also reacts to β-cell islets and whole insulin. By adding the insulin-reactive T cell clone to a CD4+ T cell panel, a positive control clone determine in a definitive fashion whether there is insulin present within any antigenic fractions for the BDC panel of clones. Alternatively, spiking fractions with whole insulin before chromatographic separation, can also determine where insulin elutes. In one embodiment, the present invention contemplates a method comprising a T cell panel comprising at least one non-insulin reactive CD4+ T cell clone.

The isolation and purification step is completed by further fractionation using gel electrophoresis (i.e., for example, either one-dimensional or two-dimensional). For example, the antigenic fractions resultant from the combined SEC/IEX chromatographic separations are assessed for purity and then further fractionated by gel electrophoresis. It is expected that antigenic fractions from SEC/IEX chromatography yields only a few bands on the electrophoretic gels. Gel lanes may be assayed for T cell clone antigenic activity by elution and/or direct protein assay. In one embodiment, the present invention contemplates a method comprising improved recovery of protein from polyacrylamide gels and preventing denaturation. In one embodiment, the improved method comprises electroelution. In one embodiment, the improved method comprises pulverizing gel slices and presenting the gel slices to macrophages for subsequent T cell clones presentation. Nonetheless, SEC/IEX followed by gel electrophoresis has been successfully decreased the candidate proteins to be analyzed by mass spectrometry.

B. Mass Spectrometry

In one embodiment, the present invention contemplates a method comprising unambiguously identifying protein antigens by using a modified mass spectrometry technique. (26, 27). Briefly, candidate proteins may be excised from an electrophoretic gel either individually or in regions. Subsequently, the gel samples may be further processed by destaining, reducing (i.e., for example, by using dithiothreitol (DTT) or tris-(2-carboxyethyl)phosphine, hydrochloride (TCEP)) and/or alkylating (i.e., for example, by using iodoacetamide). Processed gel bands and/or regions can then be digested with trypsin overnight and fragmented peptides extracted from the gels and process using a speed vacuum to reduce volume and remove residual organic solvents. Peptides will be chromatographically resolved on-line using a C18 column and analyzed using an ion trap mass spectrometer.

In one embodiment, the mass spectrometry system includes, but is not limited to, a high performance liquid chromatography (HPLC) chip interface, a relatively new technology that enables fairly rapid analysis of complex samples due to a decrease in dead volume (Lin, J., Reisdorph, N., et al, manuscript submitted). The ion trap is equipped with both collision-induced and electron transfer dissociation for fragmentation. Using alternate forms of fragmentation will conceivably result in better overall sequence coverage of peptides, ultimately improving confidence in protein identification.

Data can be searched using a Spectrum Mill® search engine (Rev A.03.01.037 SR1, Agilent Technologies, Palo Alto, Calif.), for which confidence thresholds include peptide scores of at least 10 and Scored Percent Intensity of at least 70%. A reverse (random) database search will be simultaneously performed to generate a false positive rate. Manual inspection of spectra will be performed in order to validate the match of the spectrum to the predicted peptide fragmentation pattern, hence increasing confidence in the identification. Standards are run at the beginning of each day and at the end of a set of analyses for quality control purposes.

C. Identified Protein Validation

Identified proteins are then validated. For example, antibodies against specific proteins may be used. Alternatively, Western blotting may also confirm the mass spectrometry results. Further, validation can be performed using 1D or 2D gels. Another way to validate antigen identification may utilize a commercially available source of the identified protein and compare antigenicity with T cell clones assays. In some cases, the identified protein may be recombinantly cloned and expressed in order to verify its antigenicity. Once a positive identification has been made, validation may be accomplished using a QTOF mass spectrometer.

D. Advantages Of the Improved Isolation And Purification Techniques

As described above, many of the problems plagued efforts to identify T cell antigens in the past. One limitation was obtaining a sufficient source of beta cell antigens. (23) The improvement described herein: (a) established that the tumor cell lines could be grown in culture without losing antigenicity; (b) determined yield optimization of fresh adenomas and stockpiling material for later use; (c) minimized loss of antigenicity during sample handling and storage, (d) determined a minimal threshold amount (e.g., >1 mg) of total membrane protein to consistently detect antigenic material in column chromatography fractions. Further, these improvements were a reflection of a steady source of beta cell adenoma material provided by improved methods of maintaining the transgenic NOD RIP-Tag mice. Unlike previous publication, the present invention contemplates a method for routine breeding of NOD RIP-Tag mice, thereby generating sufficient whole beta cell membrane material (i.e., for example, 3-5 mg for each analysis).

The data presented herein was determined by using the improved biochemical techniques to isolate antigens capable of activating a panel of diabetogenic autoreactive CD4+ T cell clones. Further refinements to extend chromatographic separation procedures to achieve a greater enrichment and purification of antigen than is indicated by 40-50 proteins by SDS-PAGE, may be possible by further eliminating the number of candidate proteins, thereby facilitating subsequent mass spectrometric analysis. Increases in resolution of the antigenic fractions may also be possible by altering the salt gradient (e.g., by using a combination of step and linear gradients) to change the elution pattern after IEX. Optimizing a molecular weight cut-off procedure wherein significant antigenic activity is retained, will result in further removal of non-antigenic proteins.

Of course, there are additional or alternative chromatographic methods that could be employed to increase resolution of the antigenic fractions. These include, but are not limited to, chromatofocusing (based on pI) and Cation Exchange (e.g. HiTrap). Alternatively, column chemistries that separate intact proteins on a C18 column (reverse phase chromatography) are adaptable to the presently described experimental design. Adaptations must be carefully assessed however, to ensure that the delectability of an antigen is improved. For example, if concentrations of detergent or salt in eluted fractions are too high T cell responses are reduced, reflecting a reduction in detectable antigenic proteins. For this particular problem, one embodiment of the present invention contemplates using low concentrations of Tween 20 in elution buffers to displace OβG and/or by using dialysis to decrease salt concentration or detergent.

Problems in identifying low abundance proteins with mass spectrometry analysis were improved using an ion trap mass spectrometer. If sensitivity for individual proteins may be further improved by performing in-solution digests. In one embodiment, the ion trap mass spectrometer is equipped with an electron transfer dissociation and a collision induced dissociation. Although it is not necessary to understand the mechanism of an invention, it is believed that by using both types of fragmentation overall coverage of a protein may be increased, thereby increasing identification confidence levels.

### VII. Differential Proteomics Analysis

In one embodiment, the present invention contemplates a method comprising using differential proteomic analysis to identify T cell antigen candidates in beta tumor cells. In one embodiment, the method further comprises determining antigen activity with T cell clones.

Differential analysis comprises an alternative method of identifying potential antigens, either as a complement to, or instead of, the improved biochemical techniques described above. One improvement was related to observations that antigen(s) capable of activating a diabetogenic T cell clone panel, were not well passed using conventional cell culturing techniques of beta tumor cell lines. (22). Breeding the NOD RIP-Tag mice to generate a steady source of antigenic material is one example of new techniques to over come this problem. Consequently, these refined techniques consistently obtain crude membrane preparations from fresh tumors with a high degree of antigenic activity. These preparations are also starting material for proteomic analyses.

A beta cell adenoma cell line (NIT-1) was derived from the NOD RIP-Tag mouse and has served as an antigen-negative counterpart as neither whole NIT-1 cells nor NIT-1 lysates are antigenic. (24). Because the NIT-1 and antigen positive cells are so closely related, some protein(s) present in highly purified material from RIP-Tag tumor cells that are not present in purified NIT-1 lysates may be a BDC-2.5 antigen. Following tumor cell lysis and chromatographic separation, several bands in SDS gels appear that differ between preparations of fresh RIP-Tag tumors and NIT-1 cells. See, FIG. 3. Consequently, these cells were chosen as a starting point for identifying proteins unique to these antigenic tumor cells.

Two-dimensional gel electrophoresis (2DGE) has been used successfully by many laboratories for analyzing differential protein expression from several biological sources. (27). Using this technique, upwards of 2,000 proteins can be separated on a large format gel and ˜300 proteins on a small gel. Sophisticated software may be used to detect proteins and to determine relative changes in protein abundance. When combined with labeling technologies, as with Differential Gel Electrophoresis (DiGE), there is an increased potential to minimize variability and to perform statistical analysis. When used properly, 2DGE is a powerful quantitative proteomics technique.

In one embodiment, the present invention contemplates a method comprising 2DGE/DIGE capable of partially enriching samples to facilitate identifying antigen candidate proteins. Although it is not necessary to understand the mechanism of an invention, it is believed that 2DGE/DIGE is an important advantage in optimizing proteomic differential analysis because by starting with partially purified fractions, many contaminating non-antigenic proteins do not migrate with the antigenic bands. In addition to providing a strategy for identifying antigen, 2DGE provides an opportunity to visualize differences in proteins or abundance due to post-translational modification or otherwise similar protein isoforms. 2DGE can also be used to dramatically increase the resolution of proteins previously separated on 1D gels.

In one embodiment, the present invention contemplates a method comprising the isolation of at least one protein using 2DGE/DiGE that corresponds to an antigen present on RIPTag 1D gels, but not on NIT-1 1D gels. In one embodiment, the method isolates a plurality of proteins that are on RIPTag 1D gels, but not on NIT-1 1D gels. In one embodiment, an antigen appearing on both the RIPTag and NIT-1 1D gels is slightly modified in the RIPTag 1D gel. In one embodiment, an antigen appearing on both the RIPTag and NIT-1 1D gels is slightly modified in the NIT-1 1D gel.

In one embodiment, the present invention contemplates a method comprising using mass spectrometry on 2DGE/DiGE isolated proteins to identify post translational modifications including, but not limited to: i) phosphorylations (28-30); ii) ubiquitinations (31); and iii) structural differences (i.e., for example, disulfides). In one embodiment, the present invention contemplates a method comprising improving solubility of hydrophobic proteins (i.e., for example, membrane proteins) such that the proteins are absorbed into a first dimension acrylamide gel matrix. In one embodiment, the method further comprises performing a quantitative LC/MS/MS approach using ICAT or iTRAQ labeling. (32). These methods are gel-free can quantitatively analyze membrane proteins and can be used as a means of validating 2DGE results. Another validation method comprises using a QTOF mass spectrometer.

Where an antigen is expressed in altered forms in various cell types, Western blotting is expected to differentiate between in expression in RIPTag but not NIT-1 lysates. If the antigen is present in NIT-1 lysates, then it is possible that the antigen is in an altered form. Such an altered form identify by Western Blot would be sequenced using mass spectrometry and/or map post translational modifications (see below). If Western blotting shows that the candidate antigen is indeed present in NIT1 cells (i.e., for example, non-antigenic peptide), then a determine if the proteins are actually isoforms, can be resolved separately using 2DGE and antigen candidate proteins will be excised and digested.

Tandem mass spectrometry can then be used to determine the sequence of the two isoform proteins and also to map modifications. A Coomassie-stained gel slice from a 2D gel is believed sufficient to obtain at least a 50% sequence, while other. strategies can be used to improve chances of obtaining a 100% sequence, such as: 1) scaled up the amount of protein (i.e., for example, RT-PCR), 2) multiple protease fragmentation (i.e., for example, trypsin, chymotrypsin, Glu-C), 3) optimization of the LC/MS portion, 4) bimodal mass spectrometry fragmentation with an ion trap.

### VIII. Expression And Cloning

In one embodiment, the present invention contemplates a method comprising cloning and expressing full-length or fragmented forms of candidate antigenic proteins for confirmation of their specific immunogenicity in accordance with Example IV.

The improved isolation and purification techniques described above identify a limited number of candidate autoantigens for each of the individual T cell clones. In one embodiment, methods comprising cloning and expression of an antigenic protein provides identification of a single antigenic protein. In one embodiment, the method further comprises generating cDNAs encoding the antigenic proteins purified by the above biochemical techniques, wherein each cDNA encoding a specific protein is individually incorporating into an expression platform. This allows expression of a single antigen for uptake and processing by an APC and antigenicity testing by T cell clones stimulation.

Cloning and expression of putative autoantigen peptides can confirm that a single member of the previously defined candidates for each clone is a bona fide autoantigen. Two potential problems that might arise are if the bacterially expressed proteins are insoluble or mis-folded, or if some component of the insect cells is mitogenic for one or more of the T cells. The first problem may be solved by either using an alternative fusion partner (i.e., for example, maltose binding protein or a His tag) or expression of the protein in insect cells. Further, if the protein is secreted, a nickel agarose affinity column may be used to purify the protein from the culture medium. If insect cells cannot be used directly, a polyhistidine affinity tag may be used to purify the antigenic protein.

### IX. Immunoprecipitation

Immunoprecipitation (IP) is a technique of precipitating a protein antigen out of solution using an antibody that specifically binds to that particular protein. This process can be used to isolate and concentrate a particular protein from a sample containing many thousands of different proteins. Immunoprecipitation is usually performed with an antibody coupled to a solid substrate at some point in the procedure. Other procedures also include precipitating an autoantibody with: i) another antibody or complexed to a bead; or ii) a physical precipitation of the antigen/antibody complex by a precipitating agent such as polyethylene glycol or ammonium sulfate.

Immunoprecipitation can be used to detect an antibody (i.e., for example, a diabetogenic autoantibody) that specifically targets a single known protein (i.e., for example, a chromogranin A derived protein). To facilitate identification of the antibody-protein complex, the protein may be tagged on either the C-terminal or N-terminal end of the protein of interest. The advantage here is that the same tag can be used time and again on many different proteins while screening different antibodies. Examples of tags may include, but are not limited to, the Green Fluorescent Protein (GFP) tag, Glutathione-S-transferase (GST) tag, the FLAG-tag tag, an enzyme such as horseradish peroxidase or β-galactosidase, a luciferase (firefly, Renilla or Gluc), a chemiluminescent substrate, or a Europium complex. Alternatively, a protein may be tagged with a radioactive label (i.e., for example, ³⁵S, ³H, ¹⁴C, or ³²P).

Antibodies that are specific for a particular protein (or group of proteins) may be immobilized on a solid-phase substrate such as a superparamagnetic substrate or on an agarose substrate. The substrates with bound antibodies are then added to the protein mixture and the proteins that are targeted by the antibodies are captured onto the substrate via the antibodies (i.e., immunoprecipitated). Historically, a solid-phase support for immunoprecipitation has preferably been highly-porous agarose substrates (i.e., for example, agarose resins or slurries). The advantage with this technology is a very high potential binding capacity as virtually the entire sponge-like structure of the agarose particle is available for binding antibodies which will in turn bind the target proteins. This advantage of extremely high binding capacity must be balanced with the quantity of antibody expected to contact the agarose beads. For example, one may calculate backward from the amount of protein that needs to be captured, to amount of antibody that is required to bind that quantity of protein, and back still further to the quantity of agarose that is needed to bind that particular quantity of antibody. The portion of the binding capacity of the agarose beads that is not coated with antibody will then participate in non-specific binding events. This results in an elevated level of random non-specifically bound proteins to the substrate which results in an elevated background signal that can make it more difficult to interpret results. For these reasons it is prudent to match the quantity of agarose (in terms of binding capacity) to the quantity of antibody that one wishes to be bound for the immunoprecipitation.

Alternatively, in contrast to the direct binding methods described above (which have an inherent disadvantage of requiring the tedious procedure of coupling each and every sample to a solid substrate) indirect binding assays may also be performed where an antibody complex is formed in solution with a labeled known antigen in the presence of an unknown amount antibody (i.e., for example, an autoantibody). The antigen/antibody binding complex may then be recovered by precipitating the solution with an agent such as protein A or an antibody that recognizes all human immunoglobulins.

Once a solid substrate has been chosen, antibodies can be coupled to the substrate by, for, example, contacting the substrate with a biological sample. Next, the antibody-coated-substrate can be contacted with a labeled protein sample (i.e., for example, a labeled antigen comprising a protein epitope). At this point, antibodies that are stuck to the substrate will bind the labeled proteins for which they have specific affinity thereby completing the immunoprecipitation step. Next, the substrate is washed such that only the bound antibody-protein complex remains.

With an agarose substrate the washing steps may be accompanied by pelleting out the agarose from the residual sample by briefly spinning in a centrifuge with forces between 600-3,000×g (times the standard gravitational force). This step may be performed in a standard microcentrifuge tube, but for faster separations, greater consistency and higher recoveries, the process is often performed in small spin columns with a pore size that allows liquid, but not agarose beads to pass through. After centrifugation, the agarose substrate may form a very loose fluffy pellet at the bottom of the tube.

Following the initial capture of a protein or protein complex, the solid support may be washed several times to remove any proteins not specifically and tightly bound to the support through the antibody. After washing, the precipitated protein(s) may be eluted and analyzed using scintillation counting, gel electrophoresis, mass spectrometry, western blotting, or any number of other methods for identifying constituents in the complex.

### X. Therapeutic Applications

Proteomic analysis of pancreatic β-cells is believed to identify previously unrecognized components that are antigenic for diabetogenic T cells. Autoreactive diabetogenic T cells are considered primary mediators in the initiation and propagation of the autoimmune diabetes disease process. Knowledge of these autoantigens that activate autoreactive T cells will allow us to determine if the orthologous molecules are also targeted in the human disease. Although previous attempts at identifying T cell autoantigens have been hampered by either lack of an appropriate biological system or limitations of technology, this proposal is timely in that both of these vital pieces are finally in place.

A. T Cell Autoantigens

There have been various treatments aimed at autoreactive T cells, but to date few of these are appropriate for use in humans. In one embodiment, the present invention contemplates a method for treating a diabetic patient comprising providing an autogenic T cell peptide autoantigen. Although it is not necessary to understand the mechanism of an invention, it is believed that autogenic T cell peptide autoantigens will allow improved T1D therapeutic intervention at the level of the responsible autoreactive T cell. In some embodiments, the autoantigenic peptides are used to generate monoclonal therapeutic antibodies. In some embodiments, the autoantigenic peptides are used as screening targets to identify antidiabetes drugs. In some embodiments, the autoantigenic peptides are used in methods for early diagnosis of diabetes and monitoring of diabetes progression.

In one embodiment, the present invention contemplates autoantigens for pathogenic T cells in NOD mice that are also antigenic in humans. Although it is not necessary to understand the mechanism of an invention, it is believed that post-translationally modified peptides from the secretory granule protein chromogranin A (ChgA) may provide functional ligands for diabetogenic T cells in T1D. In one embodiment, the present invention contemplates a method comprising activating human T cells using ChgA peptide sequences known to be antigenic for NOD-derived diabetogenic T cell clones. In one embodiment, the antigenic activation of human T cells is modulated by ChgA peptide posttranslational modifications.

In one embodiment, the present invention contemplates a method comprising stimulating T cells derived from established or new onset T1D human patients using ChgA peptide as described herein. In one embodiment, the present invention contemplates a human autoantigen comprising an amino acid sequence comprising at least a portion of a ChgA-like peptide. In one embodiment, the human autoantigen is associated with autoimmune disease. In one embodiment, the autoimmune disease including but not limited to diabetes, arthritis, or Chron's disease. Although it is not necessary to understand the mechanism of an invention, it is believed that until the present invention, ChgA has not been identified as an autoantigen in any disease.

B. T Cell Tolerization

In one embodiment, the present invention contemplates a method comprising promoting expansion of regulatory T cells by activation with ChgA peptide epitopes. Although it is not necessary to understand the mechanism of an invention, it is believed that such T cell expansion (i.e., for example, activation) can restore tolerance to pancreatic β-cells. In some embodiments, the ChgA epitopes comprise autoimmune biomarkers that can be monitored to provide insight into the progression of any autoimmune disease, or efficacy of therapeutic interventions.

In one embodiment, the present invention contemplates tolerizing autoreactive T cells using ChgA peptide fragments. It has been reported that NOD mice T cells may be tolerized with peptides of various candidate antigens, especially insulin and GAD. As has been noted previously, the choice of peptide, route of administration, and other factors can greatly influence the outcome of such studies. Hutchings et al., “Protection from insulin dependent diabetes mellitus afforded by insulin antigens in incomplete Freund's adjuvant depends on route of administration” J Autoimmun 11:127 (1998): and von Herrath et al., “Tolerance induction with agonist peptides recognized by autoaggressive lymphocytes is transient: therapeutic potential for type 1 diabetes is limited and depends on time-point of administration, choice of epitope and adjuvant” J Autoimmun 16:193 (2001). Tolerance induction may be approached in at least two methods: i) administration of a peptide in adjuvant; and ii) administration of an antigen cross-linked to an antigen presenting cell.

1. Subcutaneous Immunization

One approach to generate antigen-specific tolerization comprises subcutaneous administration of peptide in Incomplete Freund's Adjuvant (IFA). Insulin B chain 9-23 can serve as a positive control, whereas an insulin A chain can serve as a negative control. Insulin A chain has previously been shown to be non-protective and/or a non-antigenic ChgA peptide. Diabetes can be induced in approximately 4-6 weeks by transfer into healthy mice of diabetogenic T cells from T cell receptor transgenic mice, in which the T cells have the same autoreactivity as one of the diabetogenic T cell clones, or spleen cells from diabetic NOD donors. Mice are then monitored weekly for changes in urine/blood glucose. Using this model it can be determined if spontaneous NOD diabetes can be delayed or prevented. For example, at different time points after treatment, some animals from each group will be sacrificed to determine whether T cell numbers and phenotype in the pancreas change under the tolerogenic protocol. Exemplary observations, including but not limited to, whether effector Th1 cells decrease in number or functional activity or whether Tregs increase.

2. Splenocyte Coupling

It has been reported that ethylenecarbodiimide (ECDI)-fixed antigen presenting cells may prevent experimental autoimmune encephalomyelitis (EAE). Miller et al., “Antigen-specific tolerance as a therapy for experimental autoimmune encephalomyelitis” Int Rev Immunol 9:203 (1992); Turley et al., “Peripheral tolerance induction using ethylenecarbodiimide-fixed APCs uses both direct and indirect mechanisms of antigen presentation for prevention of experimental autoimmune encephalomyelitis” J Immunol 178:2212 (2007); and Miller et al., “Antigen-specific tolerance strategies for the prevention and treatment of autoimmune disease” Nat Rev Immunol 7:665 (2007). In one embodiment, the present invention contemplates methods for testing ChgA peptide fragments (i.e., for example, WE14), in unmodified and enzymatically converted forms to induce T cell tolerance. In one embodiment, T cell tolerance is induced in NOD mice. In one embodiment, T cell tolerance is induced in humans. For example, spleen cell suspensions may be coupled with peptides using ECDI and then the peptide-coupled cells are administered intravenously. This testing regime is useful in adoptive transfer models as well as in spontaneous disease. Alternative approaches including but not limited to, peptide fragment administration through mucosal pathways.

Such tolerance-inducing regimes are compatible with accelerated disease induction models and also in unmanipulated NOD mice to determine whether spontaneous disease can be delayed or prevented. Tolerance induction studies could also include combination therapy approaches, e.g., anti-CD3 in addition to peptide or peptide complexed to MHC molecules or antigen-presenting cells.

A variety of tolerance induction strategies that target autoreactive T cells, particularly those involving combinational approaches, have been found to be effective in preventing and/or reversing T1D. These include, but are not limited to, treatment with anti-CD3 and/or insulin. However, singling out specific T cell subsets based on TCR specificity has been difficult, partly due to the few well-characterized T cell specificities available for study in T1D. The identification of new beta cell target antigens allow tests as to whether pathogenic T cells reactive for this antigen can be “turned off” or, alternatively, whether regulatory T cells (Tregs) with similar specificity and which act to suppress inflammation can be induced. Such studies can be carried out in the non-obese diabetic (NOD) mouse which develops type 1 diabetes spontaneously. Since at least one autoreactive T cell clone that responds to diabetogenic autoantigens is the well-known highly diabetogenic BDC-2.5 clone and/or T cells in the BDC-2.5 TCR/NOD transgenic mouse, in vivo investigations may be performed.

One approach is to develop antigen-specific therapy for T1D based on peptide fragments of Chromogranin A, as described herein. For example, a peptide ligand may be used to establish tolerance induction in T cells. Although it is not necessary to understand the mechanism of an invention, it is believed that the natural ligand of protein antigens, which may also be a natural cleavage product of the wild type protein and found in various cell types, only becomes antigenic upon enzymatic conversion (i.e., for example, a post-translational modification). It is believed that such enzymatic conversions may occur under conditions of increased pancreatic beta cell stress. Natural amino acid sequences of a chromogranin A peptide (i.e., for example, WS[R/K]MDQLAKELTAE (SEQ ID NO: 54)) or a post-translationally modified version of the peptide are believed to be the most effective form of the peptide to use in tolerance induction protocols. The data presented herein shows that the natural mouse sequence WSRMDQLAKELTAE (SEQ ID NO: 11) administered to NOD mice can suppress pathogenic T cell activity, both in vitro and in vivo.

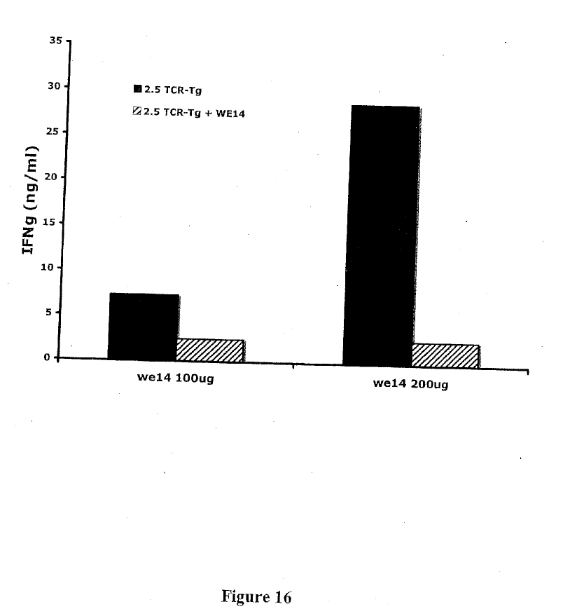

In one experiment, BDC-2.5 TCR-Tg NOD mice were immunized with WE14 (50 mg) in complete Freund's adjuvant (CFA) at day 0 and boosted with WE14 in incomplete Freund's adjuvant (IFA) at day 7. Spleen cells were harvested 14 days after the initial immunization and assayed by ELISA with WE14 (100 mg or 200 mg) as antigen. The results indicate that the IFNγ response of the diabetogenic T cells was considerably reduced after WE14 immunization. See, FIG. 16.

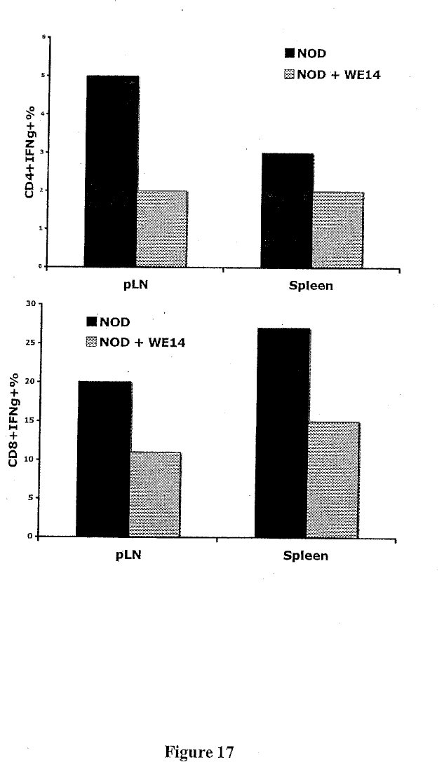

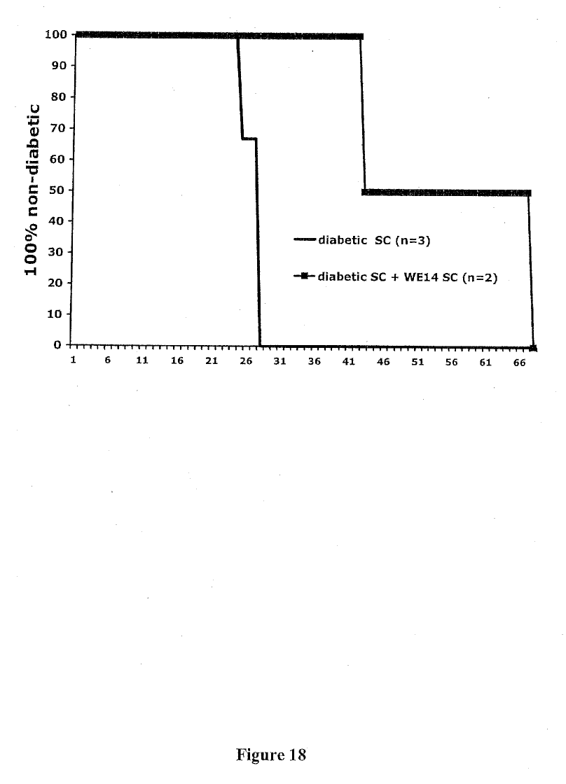

In another experiment, NOD mice (3-4 wks old) were immunized intraperitoneally (i.p.) with WE14 (100 mg+IFA) at day 0 and boosted with the same dose of peptide 30 days later. Seven weeks after the initial immunization, pancreatic lymph nodes (pLN) and spleen were harvested and single cell suspensions of these organs were made. The cells were stimulated with anti-CD3 (2 mg/ml) and anti-CD28 (2 mg/ml) for 48 hr. PMA/ionomycin and Golgiplug were added for an additional 4 hr. Cells were harvested and analyzed for intracellular IFNγ production by flow cytometry. The results show that immunization of NOD mice with the WE14 peptide can suppress the inflammatory response of both CD4+ and CD8+ T cells in the lymphoid organs of these mice. See, FIG. 17.

In another experiment, NOD mice (3-4 wks old) were immunized with WE14+IFA and 13 days later, single cell suspensions of the spleens (WE14 SC) were prepared. WE14 SC (1×10⁷) were co-transferred with spleen cells obtained from a diabetic NOD mouse (1×10⁷) into adult NOD.scid recipients by Intravenous (i.v.) injection. Urine glucose was monitored daily following cell transfer and hyperglycemia was confirmed by blood glucose readings. This data suggest that disease induced by diabetic NOD spleen cells can be considerably delayed in the presence of T cells from a mouse immunized with WE14 peptide.

### XI. Antibody Generation

The present invention provides isolated antibodies (i.e., for example, polyclonal or monoclonal). In one embodiment, the present invention provides monoclonal antibodies that specifically bind to a chromogranin A protein fragment as described herein. These antibodies find use in detection, diagnostic, and therapeutic methods as described above.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a protein expressed resulting from a virus infection (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### XII. Pharmaceutical Compositions

The present invention further provides pharmaceutical compositions (e.g., comprising the small molecule inhibitors, antisense, or antibody compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antibody compounds and (b) one or more other therapeutic compounds that function by a non-immune mechanism. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of therapeutic compound accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### XIII. Kits

In another embodiment, the present invention contemplates kits for the practice of the methods of this invention. The kits preferably include one or more containers containing a . . . method of this invention. The kit can optionally include a first container comprising a panel comprising at least two CD4+ Th1 T cell clones. The kit can optionally include a plurality of containers comprising buffers and reagents capable of maintaining the at least two clones. The kit can optionally include a container comprising a monoclonal antibody directed to a diabetogenic autoantigen. The kit can optionally include enzymes capable of performing PCR (i.e., for example, DNA polymerase, Taq polymerase and/or restriction enzymes). The kit can optionally include a pharmaceutically acceptable excipient and/or a delivery vehicle (e.g., a liposome). The reagents may be provided suspended in the excipient and/or delivery vehicle or may be provided as a separate component which can be later combined with the excipient and/or delivery vehicle. The kit may optionally contain additional therapeutics to be co-administered with the monoclonal antibody.

The kits may also optionally include appropriate systems (e.g. opaque containers) or stabilizers (e.g. antioxidants) to prevent degradation of the reagents by light or other adverse conditions.

The kits may optionally include instructional materials containing directions (i.e., protocols) providing for the use of the reagents in the detection of diabetogenic autoantigens or therapeutic administration of therapeutic agents inhibiting the activity of autoreactive diabetogenic T cells. In particular the disease can include any one or more of the disorders described herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### IVX. Detection Methodologies

A. Detection of Nucleic Acids mRNA expression may be measured by any suitable method, including but not limited to, those disclosed below.

In some embodiments, RNA is detection by Northern blot analysis. Northern blot analysis involves the separation of RNA and hybridization of a complementary labeled probe.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to a oligonucleotide probe. A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

B. Sequencing Of Nucleic Acids

The method most commonly used as the basis for nucleic acid sequencing, or for identifying a target base, is the enzymatic chain-termination method of Sanger. Traditionally, such methods relied on gel electrophoresis to resolve, according to their size, wherein nucleic acid fragments are produced from a larger nucleic acid segment. However, in recent years various sequencing technologies have evolved which rely on a range of different detection strategies, such as mass spectrometry and array technologies.

One class of sequencing methods assuming importance in the art are those which rely upon the detection of PPi release as the detection strategy. It has been found that such methods lend themselves admirably to large scale genomic projects or clinical sequencing or screening, where relatively cost-effective units with high throughput are needed.

Methods of sequencing based on the concept of detecting inorganic pyrophosphate (PPi) which is released during a polymerase reaction have been described in the literature for example (WO 93/23564, WO 89/09283, WO98/13523 and WO 98/28440). As each nucleotide is added to a growing nucleic acid strand during a polymerase reaction, a pyrophosphate molecule is released. It has been found that pyrophosphate released under these conditions can readily be detected, for example enzymically e.g. by the generation of light in the luciferase-luciferin reaction. Such methods enable a base to be identified in a target position and DNA to be sequenced simply and rapidly whilst avoiding the need for electrophoresis and the use of labels.

At its most basic, a PPi-based sequencing reaction involves simply carrying out a primer-directed polymerase extension reaction, and detecting whether or not that nucleotide has been incorporated by detecting whether or not PPi has been released. Conveniently, this detection of PPi-release may be achieved enzymatically, and most conveniently by means of a luciferase-based light detection reaction termed ELIDA.

It has been found that dATP added as a nucleotide for incorporation, interferes with the luciferase reaction used for PPi detection. Accordingly, a major improvement to the basic PPi-based sequencing method has been to use, in place of dATP, a dATP analogue (specifically dATPαs) which is incapable of acting as a substrate for luciferase, but which is nonetheless capable of being incorporated into a nucleotide chain by a polymerase enzyme (WO98/13523).

Further improvements to the basic PPi-based sequencing technique include the use of a nucleotide degrading enzyme such as apyrase during the polymerase step, so that unincorporated nucleotides are degraded, as described in WO 98/28440, and the use of a single-stranded nucleic acid binding protein in the reaction mixture after annealing of the primers to the template, which has been found to have a beneficial effect in reducing the number of false signals, as described in WO 00/43540.

C. Detection of Protein

In other embodiments, gene expression may be detected by measuring the expression of a protein or polypeptide. Protein expression may be detected by any suitable method. In some embodiments, proteins are detected by immunohistochemistry. In other embodiments, proteins are detected by their binding to an antibody raised against the protein. The generation of antibodies is described herein.

Antibody binding may be detected by many different techniques including, but not limited to, (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

D. Remote Detection Systems

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, wherein the information is provided to medical personal and/or subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

E. Detection Kits

In other embodiments, the present invention provides kits for the detection and characterization of proteins and/or nucleic acids. In some embodiments, the kits contain antibodies specific for a protein expressed from a gene of interest, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

### XV. Therapeutic Agent Delivery Systems

The present invention contemplates several therapeutic agent delivery systems that provide for roughly uniform distribution, have controllable rates of release. A variety of different media are described below that are useful in creating therapeutic agent delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One embodiment of the present invention contemplates a delivery system comprising therapeutic agents as described herein.

### Microparticles

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

Liposomes

One embodiment of the present invention contemplates liposomes capable of attaching and releasing therapeutic agents described herein. Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of at least one therapeutic agent. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze dried medium comprising at least one therapeutic agent is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of therapeutic agent release. Miller et al., Degradation Rates of Oral Resorbable Implants [Polylactates and Polyglycolates: Rate Modification and Changes in PLA/PGA Copolymer Ratios, J. Biomed. Mater. Res., Vol. 11:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of a therapeutic agent to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and therapeutic agent is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 μm and composed of biocompatible and biodegradable polymers. Specific polymer compositions of a microsphere can control the therapeutic agent release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a therapeutic agent. Preferably, these microparticles are soluble and the selected protein (i.e., fibrinogen or thrombin) creates the walls of the microparticles. Consequently, the therapeutic agents are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

### EXPERIMENTAL

### Example I

### Assay For Antigenicity

This example describes an assay of diabetogenic T cell clones from a BDC panel by a reaction with autoantigens from a pancreatic β-cell membrane preparation.

To obtain antigenic material for separation by chromatographic procedures, a crude membrane preparation was made from beta tumor cells isolated from freshly excised NOD RIPTag adenomas. Adenomas were harvested from the mice when they were about 4 months of age and processed into membrane preparations and used immediately or frozen for later use.

Initially, the RIPTag tumor cells are disrupted through a 30 gauge needle strainer and subjected to low speed centrifugation (2000×g ˜10 min) to remove cellular debris. See, FIG. 2. Subsequently, a whole-cell membrane preparation (i.e., for example, insulin granules) is obtained through high-speed centrifugation. The final pellet is either distributed into aliquots and frozen, or directly solubilized in octyl-beta glucoside (OβG)-containing lysis buffer to be further fractionated by chromatography.

In brief, the isolation procedure comprises the following steps:

| | Purification Step | Antigenic Fractions |
|---|---|---|
| 1. | Disruption of tumor cells. | Homogenate |
| 2. | Low speed centrifugation of | Supernatant (SN), |
| | RIP-Tag tumor cells | Pellet |
| 3. | Suspension of cell pellet B, | Supernatant |
| | low speed centrifugation | |
| 4. | High speed centrifugation of | Pellet |
| | supernatants B and C | |
| 5. | Resuspension of pellets D & E | Pellets |
| | High speed wash (2X) | |
| 6. | Solubilization of Pellet F | Lysate |
| | in 1% OβG | |

### Example II

### Chromatography Antigen Purification

Antigenic material can be obtained in the form of a membrane preparation made from NOD RIPTag beta tumor cells according to the methods described in accordance with Example I.

Before fractionation and throughout the chromatographic separations, samples are taken for each step to assess protein content and antigenicity. Tracking antigenicity is dependent on sensitive and reliable bioassays. For example, an IFNγ response is faster and much more reproducible and accurate than the standard T cell proliferation assay. Antigenicity for the T cell clones is detected through T cell responses to a source of antigen and NOD APC. See, FIG. 1.

The T cell clones are maintained in culture by periodic re-stimulation with irradiated NOD splenocytes and islet cells or β-membrane. To assay for antigen, resting responder T cells (i.e., for example, the cells at the end of the two-week restimulation period) are co-cultured for 24 hr with elicited peritoneal macrophages (PEC) as APC and either a test sample or control antigen. Control antigen will be in the form of islet cells or the whole-cell membrane fraction (i.e., for example, a β-membrane fraction). As described above, unlysed β-membrane is stored in aliquots at −80° C. for this purpose. Negative controls include responder cells alone and responders plus APC.

At the end of the culture period, supernatants are collected from the cultures and IFNγ production is measured by ELISA. Wells positive for IFNγ are indicative of T cells responding to antigen; the amount of cytokine produced is calculated from a standard curve and is directly proportional to the amount of antigen present.

Combined chromatographic procedures yield significant material antigenic for the T cell clone BDC-2.5 in a few fractions and on SDS-PAGE gels, 40-50 bands detectable by silver stain. See FIG. 4. Additional separation procedures and/or refinements of those currently being used consistently yield a fraction with high antigenic activity and a small number of bands (<10) on silver- or fluorescent-stained SDS gels (i.e., for example, size exclusion chromatography (SEC) followed by ion exchange chromatography (IEX)).

Antigenic protein fractions were identified after SEC. See, FIG. 3. These antigenic fractions were then further separated by IEX. See, FIG. 4. Improved resolution of the fractionation by IEX can be attained by making the salt gradient more shallow in the range at which the antigen elutes. As indicated in FIG. 3 and FIG. 4, samples from the antigenic fractions collected from each chromatography step were assayed for antigenic activity with the T cell clones prior to subsequent analysis.

Antigenic activity for BDC-2.5 elutes within a small number of fractions from size exclusion chromatography (SEC) of a beta cell membrane lysate. SEC protein profiles from membrane preparations made from fresh RIP-Tag and the NIT-1 cell line are similar but not identical. Antigenicity is detected only in RIP-Tag membrane preparations. SDS PAGE analysis of the fractions in the antigenic zone indicates that there are some differences in proteins between freshly harvested beta tumor cells and NIT-1 cells in this region.

### Example III

### Two Dimensional Gel Electrophoresis

This example, describes further isolation and enrichment of beta cell membrane proteins subsequent to chromatographic steps in accordance with Example II.

Initially, proteins are dialyzed in order to be resuspending in a buffer compatible with 2DGE. Alternatively, proteins may be precipitated with methanol/chloroform or TCA/Acetone using standard procedures. Protein samples analyzed using DiGE are processed in accordance with the manufacturers instructions and in duplicate to reduce the occurrence of falsely positive or negative results.

Briefly, RIP-TAG and NIT-1 chromatographically purified lysates will be individually labeled with Cy3 or Cy5 and a combined aliquot labeled with Cy2 as an internal standard. Dyes will be “switched” to decrease the likelihood of biased labeling and an additional “pick gel” will be used for protein identification. Lysates will be mixed in a 1:1:1 (Cy2:Cy3:Cy5) ratio and 2DGE performed. See, FIG. 7.

Approximately three hundred (300) proteins can be separated on a 11 cm 2D gel, which is capable of accommodating the quantity of proteins previously separated on a 1D gel (<50). Nonetheless, these gel protocols may be used on large gel formats to increase the sample size and/or number for more efficient processing. The first dimension analysis starts with approximately 75 μg of sample and is focused on 11 cm IPG strips pH 3-10. The second dimension provides protein separation on a 12% gel and the pick gel stained with SyproRuby®.

Comparison algorithms (i.e., for example, DeCyder Platinum® software, version 6.5; GE Healthcare; Piscataway, N.J.) are used to identify “lead” proteins. Proteins determined to be differentially regulated will be analyzed using LC/MS/MS as above. Initial validation of the identity of the candidate proteins will be performed using antibody-based methods as described above.

### Example IV

### Expression and Cloning

Full length cDNAs encoding isolated and purified antigenic peptides can be obtained, either in the form of ESTs distributed by the IMAGE consortium (ATCC), or following synthesis from mRNA isolated from insulinoma cell lines. In either case, a protein sequence obtained from mass spectrometry studies can used to generate the proper nucleotide sequence. If the protein and gene sequences are known and characterized, commercially available conventional techniques to obtaining cDNA or mRNA sequences may be utilized. In the event that the protein has never been sequenced, the peptide sequence will be reverse-translated to obtain the predicted gene sequences. For example, protein sequences obtained using tandem mass spectrometry can be used to guide and confirm the utilization of the correct gene sequence, thereby providing a modified, but straightforward, application of proteomics technologies.

After sequencing, the cDNAs are sub-cloned into an appropriate expression vector for subsequent prokaryotic or eukaryotic expression. Preferred vectors and hosts depend upon the biological characteristics of the antigenic protein for expression. For example, if the protein lacks any obvious signal peptide or transmembrane domain, or has previously been shown to be soluble, then a bacterial expression system may be appropriate. Alternatively, if a coding sequence is fused in-frame to those encoding GST (pGEX vectors Amersham), expression induced in transformed E. coli with IPTG is appropriate, wherein the fusion proteins are purified by affinity chromatography. (33).

In contrast, if an antigen appears to be a multispanning integral membrane protein then a eukaryotic system is optimal. In this case, a coding sequence can be introduced into a vector (i.e., for example, pMT/V5-His; Invitrogen) and used to transfect, for example, Drosophila Schneider S2 cells. Following induction by the addition of copper sulfate the cells will be harvested and used directly as antigen in the bioassays. Prospective antigens that appear to have a single transmembrane spanning domain can either be expressed in insect cells as described, or alternatively the probable lumenal and cytoplasmic domains could be separately expressed as GST fusion proteins in E. coli, an approach found to be successful in previous studies of islet proteins. (34).

Following confirmation of protein expression using standard Western blotting protocols with antibodies against the molecular tags, sequence of the recombinant antigens may be further verified by tandem mass spectrometry. Specifically, appropriate quantities of recombinant protein may be partially purified using antibodies against the molecular tag, the eluant further resolved on a 1D gel, and the protein digested and analyzed using mass spectrometry. When a combination of proteases is used in combination with the various fragmentation modes available on an ion trap instrument, almost complete coverage of the protein is possible. Verifying the correct amino acid sequence can demonstrate the antigenicity of the protein

The recombinant antigens may evaluated in a standard cytokine production assay as described above, in the presence and absence of antigen presenting cells, both with the cognate clone and other clones that do not recognize the native antigen, to ensure that a specific response is obtained.

### Example V

### Isolation of Autoantigenic Peptides from Nod Mice Adenomas

Identification of the autoantigens that drive pathogenic T cells in autoimmune type 1 diabetes (T1D) has long been a high priority for researchers in this field. A panel of highly diabetogenic CD4 T cell clones were isolated from the peripheral lymphoid organs of newly diabetic NOD mice. {Haskins, 1988 #6} {Haskins, 1989 #8} {Haskins, 1990 #7}. Subsequently, the BDC-2.5 clone was used to generate the BDC-2.5 T cell receptor transgenic (TCR-Tg) mouse {Katz, 1993 #12}, an animal that has been widely used to investigate pathogenesis and regulation of T1D.

The relevance of the BDC panel to autoreactive T cells in T1D has been most recently underscored by the demonstration of their highly potent activity in retrogenic mice; of particular note was the T cell clone BDC-10.1 because of its aggressive pathogenicity and the rapid development of diabetes in BDC-10.1 mice {Burton, 2008 #16}. Although the functional properties of these T cell clones have been well described {Haskins, 2005 #11}, identification of the beta cell antigen(s) to which they respond has been highly elusive. This example presents data obtained through two parallel but separate approaches converge to yield the identity of the antigen for three T cell clones from the panel—BDC-2.5, BDC-10.1, and BDC-5.10.3—as the insulin secretory granule protein, chromogranin A.

Whole mouse islet cells or cell extracts were used as antigen in the routine culture and assay of T cell clones from the BDC panel. Earlier efforts to isolate the antigens from islet beta cell adenomas through biochemical separation procedures resulted in two principal findings: (a) the antigenic activity resided in the granule portion of islet beta cells and (b) several of the T cell clones showed reactivity to the same granule fraction {Bergman, 1994 #9; Bergman, 2000 #10}. Subsequently, several BDC-2.5 peptide mimotopes containing similar amino acid motifs were described {Judkowski, 2001 #15}{Yoshida, 2002 #13}, at least one of which could also stimulate the BDC-10.1 clone {Yoshida, 2002 #13}. Heretofore, however, efforts to identify the natural peptide ligand and the protein source from which it is derived have been unsuccessful. In this study, two independently conducted experimental approaches were used to identify the autoantigen, one through chromatographic separation and mass spectrometry and the second through screening of a peptide library with T cell hybridomas.

Beta cell adenomas isolated from NOD RIP-Tag mice 0 provide an abundant source of antigen for the T cell clones. To biochemically purify the antigenic activity from beta cell adenomas, whole tumor tissue was separated into a preparation enriched in the beta granules by differential centrifugation as previously described {Bergman, 2000 #10}, and a detergent lysate of the membrane preparation was then subjected to sequential size exclusion (SEC) and either ion exchange (IEX) chromatography and/or reverse-phase high performance liquid chromatography (RP-HPLC).

Fractions from the SEC column were tested for activity with the T cell clones and the antigen-positive fractions (FIG. 1a) were pooled for further separation by IEX or RP-HPLC and assay with the T cell clones (FIG. 1b). FIG. 1c shows a representative silver-stained gel from the chromatographic separations and the relative degree of purification is summarized in a table (FIG. 1d). In solution tryptic digests of the IEX fractions with antigenic activity were subjected to mass spectrometric analysis. Peptides identified were matched to proteins using a database search (swissprot). Spectral intensities (FIG. 1e) indicate relative abundance of individual proteins identified in each fraction and a comparison of spectral intensities with antigenicity in each fraction resulted in a list of potential antigen candidates including secretogranins 1 and 2, insulin-2, insulin-like growth factor II, and chromogranin A. Only chromogranin A contained a sequence EDKRWSRMD (SEQ ID NO: 46) with homology to the peptide mimotopes HRPIWARMD (SEQ ID NO: 33) and HIPIWARMD (SEQ ID NO: 36) that was activating for BDC-2.5 and/or BDC-10.1.

In a separate approach to identify the antigen specificity of BDC-2.5, a baculovirus display system was used to generate a peptide library for I-A^{g7}. Soluble TCR was used to sort by flow cytometry peptide:MHC complexes displayed on the surface of insect cells by recombinant baculovirus. Baculovirus peptide libraries are fully randomized at all varied positions, thus differing from synthetic combinatorial peptide library systems in which individual positions are fixed to achieve the optimal mimotope. The I-A^{g7} library was sorted a total of three times to achieve a highly enriched population of BDC-2.5 TCR-binding peptides (FIG. 2a). Limiting dilution cloning yielded 48 virus clones, 46 of which bound the BDC-2.5 TCR. All of the TCR binding viruses contained one peptide sequence termed the 3 L mimotope (FIG. 2c).

A mutational analysis was performed of 3 L mimotope at positions 2 and 3 followed by their respective ability to stimulate BDC-2.5, BDC-10.1 and BDC-5.10.3 hybridomas with 3 L mimotope substituted peptides. See, Table 3.

TABLE 3
Chromogranin A Mimotope Stimulation Of
INFγ-Production

| Linked | | | |
|---|---|---|---|
| Peptide | | | |
| −1+123456789 | BDC-2.5 | BDC-10.1 | BDC-5.1.3 |
| SRLGLWVRME | + | + | + |
| (SEQ ID NO: 21) | | | |
| SRLVLWVRME | − | + | + |
| (SEQ ID NO: 22) | | | |
| SRLTLWVRME | + | + | + |
| (SEQ ID NO: 23) | | | |
| SRLSLWVRME | + | + | + |
| (SEQ ID NO: 24) | | | |
| SRLALWVRME | + | + | + |
| (SEQ ID NO: 25) | | | |
| SRLPLWVRME | + | + | + |
| (SEQ ID NO: 26) | | | |
| SRLCLWVRME | + | − | + |
| (SEQ ID NO: 27) | | | |
| SRLYLWVRME | − | + | + |
| (SEQ ID NO: 28) | | | |
| SRLRLWVRME | + | + | + |
| (SEQ ID NO: 29) | | | |
| SRLMLWVRME | − | + | + |
| (SEQ ID NO: 30) | | | |
| SRLHLWVRME | − | + | + |
| (SEQ ID NO: 31) | | | |
| SRFGLWVRME | + | + | ND |
| (SEQ ID NO: 32) | | | |

Linked peptide I-A^{g7} viruses were created containing substitutions in the 3L mimotope at positions 2 and 3. Stimulation was assessed by the upregulation of CD69 on the T cell hybridomas (3 × 10⁵/mL) after 3 hrs of co-culture with infected insect cells (1 × 10⁵/mL) in complete tumor media. A positive response (+) indicates >40% of the hybridoma population stained above a background staining of 1% for unstimulated or of the same T cell hybridoma. ND = not determined.

Like two previously identified mimotopes (Yoshida 2002), the 3 L mimotope proved to be highly cross-reactive for the three T cell hybridomas derived from diabetogenic clones BDC-2.5, BDC-5.10.3 and BDC-10.1 (FIG. 2b). Initial BLAST searches with the full 3 L mimotope revealed homology between 3 L and peptides from two self-antigens, GDP-mannose pyrophosphorylase B (Gmppb) and Dnajc14. See, FIG. 2C. However, these proteins are widely expressed, neither epitope was completely cross-reactive, and notably, these sequences were absent from the antigenic fractions of beta cell tumors See, FIG. 1. A broader BLAST search using the WXRM sequence common to other BDC-2.5 mimotopes was carried out and three candidates out of approximately 550 proteins were considered to be antigen candidates: carboxypeptidase E (Cpe) and chromogranin A (ChgA) are found in the islet granules (Brunner 2007) and one protein, kin of IRRE like 2 (Kirrel2) is beta-cell restricted (Sun 2003).

As chromogranin A was the most promising candidate identified by both the biochemical purification/proteomics analysis and the peptide library screen, peptides were synthesized with sequences identical to those of ChgA in the relevant (mimotope-like) region. The first sequence synthesized QWEDKRWSRMDQA (SEQ ID NO: 55) was to our surprise unable to stimulate the BDC-2.5 clone. Based on a literature search revealing that a peptide called WE14 (WSRMDQLAKELTAE (SEQ ID NO: 11)) is a natural cleavage product of ChgA and can be found in pancreatic islets. WE14 could stimulate the T cell clone BDC-2.5, but only very weakly when compared to whole tumor cell extract. Results of representative IFN-γ ELISAs with T cell clones, BDC-2.5, BDC-10.1, BDC-5.10.3 tested on beta cell adenoma extract (positive control), WE14 and WE14 variants; BDC-5.2.9 (from the BDC panel) and insulin-reactive clone PD 12-4.4 {Wegmann, 1994 #17} were included as negative controls. FIG. 3.

### Example VI

### Mice Husbandry

NOD and NOD RIPTag mice were bred and maintained in the Biological Resource Center at National Jewish Health, Denver Colo. ChgA^{-/-} mice (ChgA^{+/-} background strain 129/SvJ backcrossed to C57BL/6J) were generated in the animal facilities at the University of California, San Diego. Mahapatra et al., “Hypertension from targeted ablation of chromogranin A can be rescued by the human ortholog” J Clin Invest 115:1942-52 (2005).

### Example VII

### Antigen Purification and Mass Spectrometric Analysis

Enrichment of membrane proteins from beta cells isolated from NOD RIPTAg adenomas has been previously described. Bergman et al., “Biochemical characterization of a beta cell membrane fraction antigenic for autoreactive T cell clones” J Autoimmun 14: 343-51 (2000). Membrane protein preparations were solubilized for 1 h at 4° C. in detergent-containing buffer (20 mM Tris pH 8.0, 1% Octyl-β-Glucoside) followed by centrifugation at 18,400×g, (10 min, 4° C.) to remove insoluble debris. Protein content was determined using a Micro BCA kit (Pierce).

Size Exclusion (SE) chromatography was carried out on a Superdex™ 200 16/60 column (Amersham Biosciences) at room temperature (flow rate 1 ml/min, fraction size 1.25 ml, injection volume 2.0 ml) using SE buffer (20 mM Tris pH 8.0, 150 mM NaCl, 0.4 mM Tween 20). Peak antigenic fractions were dialyzed overnight (16 h, 20 mM Tris pH 6.5, 4° C.) using Tube-ODIALYZER™ (1K, GBiosciences) and then separated on a HiTrap™ Q HP column (GE Healthcare) at room temperature (flow rate 1 ml/min, fraction size 1.0 ml, injection volume 2.0 ml) applying a 20 min linear NaCl gradient after 10 min (Buffer A: 20 mM Tris pH 6.5, Buffer B: 20 mM Tris pH 6.5, 1 M NaCl). Fractions were concentrated and desalted on CBED spin columns (Norgen Biotek Corporation) using the protocol for acidic proteins described by the manufacturer. Tricine Tris gel electrophoresis was carried out on a 16.5% precast criterion gel (Bio-RAD) applying an initial 65 mA current for 10 min followed by a 35 mA current for 6 h. The gel was stained using SilverSNAP® stain (Thermo Scientific).

A standard protein identification strategy was performed using mass spectrometry. Shevchenko et al., “In-gel digestion for mass spectrometric characterization of proteins and proteomes” Nat Protoc 1:2856-2860 (2006). Briefly, proteins were digested with trypsin and extracted peptides were chromatographically resolved on-line using a C18 column and 1200 series high performance liquid chromatography (HPLC, Agilent Technologies) and analyzed using a 6340 LCMS ion trap mass spectrometer (Agilent Technologies, Palo Alto, Calif.). Raw data was extracted and searched against the SwissProt or NCBI databases using the Spectrum Mill search engine (Rev A.03.03.038 SR1, Agilent Technologies, Palo Alto, Calif.). Data was evaluated and protein identifications were considered significant if the following confidence thresholds were met: minimum of 2 peptides per protein, protein score >20, individual peptide scores of at least 10, and Scored Percent Intensity (SPI) of at least 70%. A reverse (random) database search was simultaneously performed and manual inspection of spectra was used to validate the match of the spectrum to the predicted peptide fragmentation pattern.

### Example VIII

### Antigen Assays

Antigenicity of islet cells, cellular and biochemical fractions, peptides, or insect cells expressing IA^{g7}-peptide constructs, was assessed through responses of T cell clones or hybridomas made by fusing T cell clones to the TCR⁻ version of T cell lymphoma, BW5147. White et al., “Two better cell lines for making hybridomas expressing specific T cell receptors” J Immunol 143:1822-1825 (1989). T cell clone cultures typically contained 2×10⁴ responder T cells, 2.5×10⁴ NOD peritoneal exudate cells (PEC) as APC, and antigen (SEC/IEX fractions, peptides, islet cells); all assays were performed with β-Mem as a positive control. IFNγ was measured by ELISA of culture supernatants. For cultures with T cell hybridomas, antigen/MHC activation was assessed by IL-2 production measured by a bioassay using the HT-2 T cell line. Walker et al., “Antigenspecific. I region-restricted interactions in vitro between tumor cell lines and T cell hybridomas” J Immunol 128:2164-2169 (1982). Synthetic peptides were either produced in the Molecular Resource Center at National Jewish Health or obtained from CHI Scientific, Maynard, Mass.

### Example IX

### Baculovirus Encoded IA^{g7}-Peptide Library

Details for creating baculovirus encoded MHCII-peptide libraries and screening these libraries have been previously described. Crawford et al., “Mimotopes for alloreactive and conventional T cells in a peptide-MHC display library” PloS Biol 2:E90 (2004); and Crawford et al., “Use of baculovirus MHC/peptide display libraries to characterize T-cell receptor ligands” Immunol Rev 210:156-170 (2006). In the case of IA^{g7} the peptide library was randomized at positions at p-1, p2, p3, p5, p7 and p9 using the codons NN[G/C]. Variations allowed at the four anchor positions were: p1:Arg/Ile (A[G/T]A), p4 and p6:Leu/Val ([T/G]TG), p9:Gly/Glu (G[G/A]A).

The PCR DNA fragment encoding the library was cloned directly into baculovirus DNA already encoding the IA^{g7} genes, attached via a linker to the N-terminus of the β chain. The ligated DNA was transfected into insect cells to produce a high titer baculovirus stock (˜10⁷ independent clones). Insect cells infected with the library at a multiplicity of infection of <1 were analyzed by flow cytometry for cells that expressed IA^{g7} (OX-6 Mab, BD-Pharmingen) and also bound a multivalent TCR reagent consisting of the soluble BDC-2.5 TCR captured by a biotinylated anti-Cα Mab, ADO-304, bound to Alexafluor-647 labeled streptavidin (Molecular Probes). Cells binding both reagents were sorted and incubated with more SF9 insect cells to expand the enriched virus. The infection, analysis and sorting enrichment were performed twice more. The virus was then cloned and insect cells infected with individual virus clones were tested as before for IA^{g7} expression and BDC-2.5 TCR binding. The peptide sequence encoded in the positive clones was determined.

### Example X

### Peptide Binding to IA^{g7}

Soluble IA^{g7} with covalently attached pHEL was treated with thrombin to cleave the linker attaching the peptide to the IA^{g7} β chain. Kozono et al., “Production of soluble MHC class II proteins with covalently bound single peptides” Nature 369:151-154 (1994). Samples (0.5 μg) were incubated with a soluble biotinylated version of pHEL, Biotin-GGGMKRHGLDNYRGYSL (SEQ ID NO: 56) (11 μM), either alone or in the presence of various concentrations of potential competitors peptides, in 15 μL of pH 5.6 buffer overnight at room temperature. The sample was diluted to 100 μL of PBS in a well of a 96-well ELISA plate coated with an anti-IA^{g7} monoclonal antibody, OX-6 (BD Pharmaceuticals). The captured IA^{g7} was washed several times with PBS and the bound bio-pHEL detected with alkaline phosphatase coupled Extravadin (Sigma) and o-nitrophenol phosphate.

### Example XI

### Immunoprecipitation

1. Lyse cells and prepare a biological sample.
2. Attach antibody to agarose by contacting with a biological sample.
3. Incubate solution with antibody against a protein of interest (i.e., for example, an Chromogranin A-derived antigen).
4. Precipitate the complex of interest by adding Protein A thereby removing it from bulk solution.
5. Wash precipitated complex several times. Centrifuge each time between washes and then remove supernatant. After final wash, remove as much supernatant as possible.
6. Elute proteins from solid support (i.e., for example, by using low-pH or SDS sample loading buffer).
7. Analyze complexes or antigens of interest. This can be done in a variety of ways:
   a. Quantitating a radioactive label using a scintillation counter.
   b. SDS-PAGE (sodium dodecyl sulfate-polyacrylamide gel electrophoresis) followed by gel staining.
   c. SDS-PAGE followed by: staining the gel, cutting out individual stained protein bands, and sequencing the proteins in the bands by MALDI-Mass Spectrometry
   d. Transfer and Western Blot using another antibody for proteins that were interacting with the antigen followed by chemiluminescent visualization.

### Example XII

### Human T-Cell Preparation

Human T-cells can be derived from PBMCs obtained after informed consent from individuals attending the Barbara Davis Center (BDC). The BDC clinic provides care for more than 2000 individuals with established T1D, and sees around 250 new-onset patients annually.

A total of approximately 100 samples may be tested including established or new onset (i.e., for example, <12 weeks post-diagnosis) diabetic patients and controls. PBMCs will be isolated by Ficoll/Histopaque density gradient centrifugation from freshly drawn blood and either used directly, or alternatively, enriched in different T-cell subsets (CD4+, CD8+, CD45RA+ naive cells, CD45RA+RO+ recently activated cells, CD45RO+ memory cells, or CD25+CD127-regulatory cells) using appropriate combinations of paramagnetic antibody affinity reagents (MACS beads; Miltenyi Biotech), and/or preparative FACS using the UCCC flow cytometry core facility. T cells from T1D patients reacting with autoantigens (e.g., insulin, GAD) are likely to be antigen-experienced and express a memory phenotype. Endl et al., “Coexpression of CD25 and OX40 (CD134) receptors delineates autoreactive T-cells in type 1 diabetes” Diabetes 55:50 (2006). Further, it has been demonstrated that differences in autoantigen reactivity between T1D patients and controls can be observed in CD45RO+ memory cells. Monti et al., “Evidence for in vivo primed and expanded autoreactive T cells as a specific feature of patients with type 1 diabetes” J Immunol 179:5785 (2007).

### Example XIII

### ChgA Peptide Epitopes as Agonists/Antagonists

This example evaluates the ability of ChgA peptides to effect spontaneous T cell responses in type 1 diabetic human subjects.

One objective determines whether amino acid sequences within a ChgA peptide, particularly amino acids that have been post-translationally modified, are targeted by the immune system in human T1D, and could therefore be potential therapeutic agents. To achieve this ELISPOT analyses can be conducted using PBMCs from a panel of control or diabetic subjects expressing HLA-DR3/DQ2 and/or -DR4/DQ8 and a small set of overlapping peptides within human chromogranin A (hChgA) that correspond to the mouse ChgA region containing the WE14 peptide and several overlapping peptides antigenic for murine pathogenic T cell clones. The human sequence of WE14 is identical to the mouse sequence except for one conservative amino acid change. For one set of analyses, peptides will be unmodified; for another set, peptides will be enzymatically converted under conditions similar to those used for conversion of murine peptides to highly antigenic antigenic epitopes.

### Example IVX

### ELISPOT Analysis

Antigen-specific T cells typically have a low frequency in peripheral blood (i.e., for example, generally in the range of 1:104-1:106) necessitating the use of highly sensitive assays for their detection. Meierhoff et al., “Cytokine detection by ELISPOT: relevance for immunological studies in type 1 diabetes” Diabetes Metab Res Rev 18:367 (2002). Moreover, T cells specific for the same epitope may be present in both the naïve and memory populations. Peterson et al., “Autoreactive and immunoregulatory T-cell subsets in insulin-dependent diabetes mellitus” Diabetologia 42:443 (1999). Differentiation may also occur in both protective and pathogenic T cell phenotypes. Arif et al., “Autoreactive T cell responses show proinflammatory polarization in diabetes but a regulatory phenotype in health” J Clin Invest 113:451 (2004); and Naik et al., “Precursor frequencies of T-cells reactive to insulin in recent onset type 1 diabetes mellitus” J Autoimmun 23:55 (2004). Reverse ELISPOT is a technique capable of measuring cytokine production from antigen-specific T-cells on a single cell level, and is currently the “gold-standard” for monitoring T-cell responses to autoantigens in PBMCs. Czerkinsky et al., “Reverse ELISPOT assay for clonal analysis of cytokine production. I. Enumeration of gammainterferon-secreting cells” J Immunol Methods 110:29 (1988); Nagata et al., “Detection of autoreactive T cells in type 1 diabetes using coded autoantigens and an immunoglobulin-free cytokine ELISPOT assay: report from the fourth immunology of diabetes society T cell workshop” Ann N Y Acad Sci 1037:10 (2004); Kalyuzhny, A. E. “Chemistry and biology of the ELISPOT assay” Methods Mol Biol 302:15 (2005); and Cox et al., “Measurement of cytokine release at the single cell level using the ELISPOT assay” Methods 38:274 (2006).

### REFERENCES

1. Rossini, A. A. 2004. Autoimmune diabetes and the circle of tolerance. Diabetes 53:267.
2. Gottlieb, P. A., and G. S. Eisenbarth. 1998. Diagnosis and treatment of pre-insulin dependent diabetes. Annu Rev Med 49:391.
3. Notkins, A. L., and A. Lernmark. 2001. Autoimmune type 1 diabetes: resolved and unresolved issues. J Clin Invest 108:1247.
4. Notkins, A. L. 2002. Immunologic and genetic factors in type 1 diabetes. J Biol Chem 277:43545.
5. Maclaren, N. K., M. S. Lan, D. Schatz, J. Malone, A. L. Notkins, and J. Krischer. 2003. Multiple autoantibodies as predictors of Type 1 diabetes in a general population. Diabetologia 46:873.
6. Krischer, J. P., D. D. Cuthbertson, L. Yu, T. Orban, N. Maclaren, R. Jackson, W. E. Winter, D. A. Schatz, J. P. Palmer, and G. S. Eisenbarth. 2003. Screening strategies for the identification of multiple antibody-positive relatives of individuals with type 1 diabetes. J Clin Endocrinol Metab 88:103.
7. Herold, K. C., W. Hagopian, J. A. Auger, E. Poumian-Ruiz, L. Taylor, D. Donaldson, S. E. Gitelman, D. M. Harlan, D. Xu, R. A. Zivin, and J. A. Bluestone. 2002. Anti-CD3 monoclonal antibody in new-onset type 1 diabetes mellitus. N Engl J Med 346:1692.
8. Herold, K. C. 2004. Achieving antigen-specific immune regulation. J Clin Invest 113:346.
9. Judkowski, V., C. Pinilla, K. Schroder, L. Tucker, N. Sarvetnick, and D. B. Wilson. 2001. Identification of MHC class II-restricted peptide ligands, including a glutamic acid decarboxylase 65 sequence, that stimulate diabetogenic T cells from transgenic BDC2.5 nonobese diabetic mice. J Immunol 166:908.
10. Yoshida, K., T. Martin, K. Yamamoto, C. Dobbs, C. Munz, N. Kamikawaji, N. Nakano, H. G. Rammensee, T. Sasazuki, K. Haskins, and H. Kikutani. 2002. Evidence for shared recognition of a peptide ligand by a diverse panel of non-obese diabetic mice-derived, islet-specific, diabetogenic T cell clones. Int Immunol 14:1439.
11. Wong, F. S., J. Karttunen, C. Dumont, L. Wen, I. Visintin, I. M. Pilip, N. Shastri, E. G. Pamer, and C. A. Janeway, Jr. 1999. Identification of an MHC class I-restricted autoantigen in type 1 diabetes by screening an organ-specific cDNA library. Nat Med 5:1026.
12. Lieberman, S. M., A. M. Evans, B. Han, T. Takaki, Y. Vinnitskaya, J. A. Caldwell, D. V. Serreze, J. Shabanowitz, D. F. Hunt, S. G. Nathenson, P. Santamaria, and T. P. DiLorenzo. 2003. Identification of the beta cell antigen targeted by a prevalent population of pathogenic CD8+ T cells in autoimmune diabetes. Proc Natl Acad Sci USA 100:8384.
13. Haskins, K., M. Portas, B. Bradley, D. Wegmann, and K. Lafferty. 1988. T-lymphocyte clone specific for pancreatic islet antigen. Diabetes 37:1444.
14. Haskins, K., M. Portas, B. Bergman, K. Lafferty, and B. Bradley. 1989. Pancreatic isletspecific T-cell clones from nonobese diabetic mice. Proc Natl Acad Sci USA 86:8000.
15. Haskins, K., and M. McDuffie. 1990. Acceleration of diabetes in young NOD mice with a CD4+ islet-specific T cell clone. Science 249:1433.
16. Haskins, K., and D. Wegmann. 1996. Diabetogenic T-cell clones. Diabetes 45:1299.
17. Katz, J. D., B. Wang, K. Haskins, C. Benoist, and D. Mathis. 1993. Following a diabetogenic T cell from genesis through pathogenesis. Cell 74:1089.
18. Pauza, M. E., C. M. Dobbs, J. He, T. Patterson, S. Wagner, B. S. Anobile, B. J. Bradley, D. Lo, and K. Haskins. 2004. T-Cell Receptor Transgenic Response to an Endogenous Polymorphic Autoantigen Determines Susceptibility to Diabetes. Diabetes 53:978.
19. Haskins, K. 2005. Pathogenic T-cell clones in autoimmune diabetes: more lessons from the NOD mouse. Adv Immunol 87:123.
20. Cantor, J., and K. Haskins. 2005. Effector function of diabetogenic CD4 Th1 T cell clones: a central role for TNF-alpha. J Immunol 175:7738.
21. Dobbs, C., and K. Haskins. 2001. Comparison of a T cell clone and of T cells from a TCR transgenic mouse: TCR transgenic T cells specific for self-antigen are atypical. J Immunol 166:2495.
22. Bergman, B., and K. Haskins. 1994. Islet-specific T-cell clones from the NOD mouse respond to beta-granule antigen. Diabetes 43:197.
23. Bergman, B., J. L. McManaman, and K. Haskins. 2000. Biochemical characterization of a beta cell membrane fraction antigenic for autoreactive T cell clones. J Autoimmun 14:343.
24. Hamaguchi, K., H. R. Gaskins, and E. H. Leiter. 1991. NIT-1, a pancreatic beta-cell line established from a transgenic NOD/Lt mouse. Diabetes 40:842.
25. Wegmann, D. R., M. Norbury-Glaser, and D. Daniel. 1994. Insulin-specific T cells are a predominant component of islet infiltrates in pre-diabetic NOD mice. Eur J Immunol 24:1853.
26. Shevchenko, A., H. Tomas, J. Havlis, J. V. Olsen, and M. Mann. 2006. In-gel digestion for mass spectrometric characterization of proteins and proteomes. Nat Protoc 1:2856.
27. Bowler, R. P., M. C. Ellison, and N. Reisdorph. 2006. Proteomics in pulmonary medicine. Chest 130:567.
28. Kange, R., U. Selditz, M. Granberg, U. Lindberg, G. Ekstrand, B. Ek, and M. Gustafsson. 2005. Comparison of different IMAC techniques used for enrichment of phosphorylated peptides. J Biomol Tech 16:91.
29. Mikesh, L. M., B. Ueberheide, A. Chi, J. J. Coon, J. E. Syka, J. Shabanowitz, and D. F. Hunt. 2006. The utility of ETD mass spectrometry in proteomic analysis. Biochim Biophys Acta 1764:1811.
30. Spickett, C. M., A. R. Pitt, N. Morrice, and W. Kolch. 2006. Proteomic analysis of phosphorylation, oxidation and nitrosylation in signal transduction. Biochim Biophys Acta 1764:1823.
31. Denis, N.J., J. Vasilescu, J. P. Lambert, J. C. Smith, and D. Figeys. 2007. Tryptic digestion of ubiquitin standards reveals an improved strategy for identifying ubiquitinated proteins by mass spectrometry. Proteomics 7:868.
32. Lund, T. C., L. B. Anderson, V. McCullar, L. Higgins, G. H. Yun, B. Grzywacz, M. R. Verneris, and J. S. Miller. 2007. iTRAQ is a useful method to screen for membranebound proteins differentially expressed in human natural killer cell types. J Proteome Res 6:644.
33. Smith, D. B., and K. S. Johnson. 1988. Single-step purification of polypeptides expressed in Escherichia coli as fusions with glutathione S-transferase. Gene 67:31.
34. Arden, S. D., T. Zahn, S. Steegers, S. Webb, B. Bergman, R. M. O'Brien, and J. C. Hutton. 1999. Molecular cloning of a pancreatic islet-specific glucose-6-phosphatase catalytic subunit-related protein. Diabetes 48:531.

We claim:

1. An isolated amino acid sequence, wherein said amino acid sequence comprises at least a portion of a chromogranin A-like peptide.

2. The isolated amino acid sequence of claim 1, wherein said sequence comprises at least a portion of said chromogranin A-like peptide.

3. The isolated amino acid sequence of claim 1, wherein said sequence comprises chromogranin A-like activity.

4. The isolated amino acid sequence of claim 1, wherein sequence comprises a human amino acid sequence of WSKMDQLAKELTAE (SEQ ID NO: 1).

5. The isolated amino acid sequence of claim 1, wherein said sequence comprises a synthetic peptide mimotope.

6. The isolated amino acid sequence of claim 1, wherein said sequence further comprises at least one post-translational enzymatic modification

7. The isolated amino acid sequence of claim 1, wherein said sequence comprises a chimeric peptide.

8. A method, comprising:
a) providing;
i) a biological sample derived from a human patient comprising at least one risk marker for type 1 diabetes, wherein said sample is suspected of comprising an amino acid sequence comprising at least a portion of a chromogranin A-like peptide;
ii) a test composition comprising isolated T cells;
b) contacting said T cells with said sample under conditions that activate said T-cells; and
c) detecting said T-cell activation, thereby diagnosing said type 1 diabetes.

9. The method of claim 8, wherein said risk marker is selected from the group consisting of an autoantibody profile, a major histocompatability complex associated with type 1 diabetes, detection of urinary glucose, and elevated blood glucose.

10. The method of claim 8, wherein said isolated T cells comprise human T cells.

11. The method of claim 8, wherein said activation is detected by a measurement selected from the group consisting of at least one cytokine and at least one T cell surface receptor.

12. The method of claim 8, wherein said amino acid sequence comprises a human amino acid sequence of WSKMDQLAKELTAE (SEQ ID NO: 1).

13. The method of claim 8, wherein said amino acid sequence comprises a modified human amino acid sequence selected from the group consisting of REWEDKRWSKMDQLAKELTA (SEQ ID NO: 2), EDKRWSKMDQLAKELTAE (SEQ ID NO: 3), EDKRWSKMDQLA (SEQ ID NO: 4), WEDKRWSKMDQLAKELTAE (SEQ ID NO: 5), WEDKRWSKMDQLAKELT (SEQ ID NO: 6), WEDKRWSKMDQLAKEL (SEQ ID NO: 7), WEDKRWSKMDQLAKE (SEQ ID NO: 8), WEDKRWSKMDQLAK (SEQ ID NO: 9), and WEDKRWSKMDQLA (SEQ ID NO: 10).

14. The method of claim 8, wherein said amino acid sequence comprises a synthetic chromogranin A peptide mimotope.

15. The method of claim 8, wherein said amino acid sequence comprises at least one post-translational enzymatic modification.

16. The method of claim 8, wherein said sample is selected from the group consisting of a whole blood sample, a plasma sample, a serum sample, a tissue sample, and a pancreatic tissue sample.

17. A method, comprising:
a) providing;
i) a biological sample derived from a patient exhibiting at least one risk marker of having type 1 diabetes, wherein said sample is suspected of comprising at least one diabetogenic biomarker;
ii) a peptide comprising specific affinity for the biomarker;
b) mixing said peptide with said sample under conditions such that said biomarker binds to said peptide, thereby forming a peptide-biomarker complex; and
c) detecting said peptide-biomarker complex, thereby diagnosing said type 1 diabetes.

18. The method of claim 17, wherein said risk marker comprises an autoantibody profile, a major histocompatability complex associated with type 1 diabetes, detection of urinary glucose, and elevated blood glucose.

19. The method of claim 17, wherein said diabetogenic biomarker is selected from the group consisting of an amino acid sequence, a nucleic acid sequence, a polysaccharide, a lipid, and an autoreactive T cell.

20. The method of claim 17, wherein patient is selected from the group consisting of a human and a non-human.

21. The method of claim 17, wherein said peptide further comprises a detectable label.

22. The method of claim 17, wherein said sample is selected from the group consisting of a whole blood sample, a plasma sample, a serum sample, a tissue sample, and a pancreatic tissue sample.

23. A method, comprising:
a) providing;
i) a biological sample derived from a patient exhibiting at least one risk marker of having type 1 diabetes, wherein said sample is suspected of comprising at least one diabetogenic biomarker;
ii) a diagnostic antibody comprising specific affinity for said at least one biomarker;
b) mixing said diagnostic antibody with said sample under conditions such that said biomarker binds to said diagnostic antibody, thereby forming a diagnostic antibody-biomarker complex; and
c) detecting said diagnostic antibody-biomarker complex, thereby diagnosing said type 1 diabetes.

24. The method of claim 23, wherein said risk marker comprises an autoantibody profile, a major histocompatability complex associated with type 1 diabetes, detection of urinary glucose, and elevated blood glucose.

25. The method of claim 23, wherein said diabetogenic biomarker is selected from the group consisting of an amino acid sequence, a nucleic acid sequence, a polysaccharide, a lipid, and an autoreactive T cell.

24. The method of claim 23, wherein patient is selected from the group consisting of a human and a non-human.

25. The method of claim 23, wherein said diagnostic antibody further comprises a detectable label.

26. The method of claim 23, wherein said sample is selected from the group consisting of a whole blood sample, a plasma sample, a serum sample, a tissue sample, and a pancreatic tissue sample.

27. A method, comprising:
a) providing;
i) a patient exhibiting at least one symptom of type 1 diabetes;
ii) a pharmaceutical composition comprising a therapeutic agent capable of reducing the at least one symptom of type 1 diabetes;
b) administering said composition to said patient under conditions such that said at least one symptom is reduced.

28. The method of claim 27, wherein said method further comprises step (c) selected from the group consisting of wherein said administering induces T cell tolerance, wherein said administering inhibits an autoantibody associated with diabetes, and wherein said administering inhibits a pancreatic beta cell surface receptor wherein said receptor has specific affinity for the autoantibody associated with diabetes.

29. The method of claim 27, wherein said therapeutic agent is selected from the group consisting of an amino acid sequence, a nucleic acid sequence, a polysaccharide, a lipid, a T cell linked to a peptide, and a small organic molecule.

30. The method of claim 29, wherein said amino acid sequence comprises an antibody having specific affinity for an amino acid sequence comprising at least a portion of a chromogranin A-like peptide.

31. The method of claim 27, wherein said composition further comprises a molecular or cellular complex.

32. The method of claim 27, wherein said patient is selected from the group consisting of a human and a non-human.

33. A kit comprising:
a) a first container comprising a composition comprising a peptide or antibody having specific affinity for a diabetogenic biomarker;
b) a plurality of containers comprising buffers and reagents capable of detecting T cell activation; and
c) a set of instructional materials describing how to detect the T cell activation after contacting the composition with a biological sample.

34. The kit of claim 33, said biological sample comprises said diabetogenic biomarker.

35. The kit of claim 34, wherein said diabetogenic biomarker is selected from the group comprising an amino acid sequence, a nucleic acid sequence, a polysaccharide, a lipid, and an autoreactive T cell.

36. The kit of claim 33, wherein said peptide or antibody comprises a detectable label.


SEQUENCE LISTING (68 sequences)

SEQ ID NO: 1 - protein, 14 aa, Homo sapiens
WSKMDQLAKELTAE

SEQ ID NO: 2 - protein, 20 aa, Artificial Sequence
  Synthetic
REWEDKRWSKMDQLAKELTA

SEQ ID NO: 3 - protein, 18 aa, Artificial Sequence
  Synthetic
EDKRWSKMDQLAKELTAE

SEQ ID NO: 4 - protein, 12 aa, Artificial Sequence
  Synthetic
EDKRWSKMDQLA

SEQ ID NO: 5 - protein, 19 aa, Artificial Sequence
  Synthetic
WEDKRWSKMDQLAKELTAE

SEQ ID NO: 6 - protein, 17 aa, Artificial Sequence
  Synthetic
WEDKRWSKMDQLAKELT

SEQ ID NO: 7 - protein, 16 aa, Artificial Sequence
  Synthetic
WEDKRWSKMDQLAKEL

SEQ ID NO: 8 - protein, 15 aa, Artificial Sequence
  Synthetic
WEDKRWSKMDQLAKE

SEQ ID NO: 9 - protein, 14 aa, Artificial Sequence
  Synthetic
WEDKRWSKMDQLAK

SEQ ID NO: 10 - protein, 13 aa, Artificial Sequence
  Synthetic
WEDKRWSKMDQLA

SEQ ID NO: 11 - protein, 14 aa, Mus musculus
WSRMDQLAKELTAE

SEQ ID NO: 12 - protein, 20 aa, Artificial Sequence
  Synthetic
REWEDKRWSRMDQLAKELTA

SEQ ID NO: 13 - protein, 18 aa, Artificial Sequence
  Synthetic
EDKRWSRMDQLAKELTAE

SEQ ID NO: 14 - protein, 12 aa, Artificial Sequence
  Synthetic
EDKRWSRMDQLA

SEQ ID NO: 15 - protein, 19 aa, Artificial Sequence
  Synthetic
WEDKRWSRMDQLAKELTAE

SEQ ID NO: 16 - protein, 17 aa, Artificial Sequence
  Synthetic
WEDKRWSRMDQLAKELT

SEQ ID NO: 17 - protein, 16 aa, Artificial Sequence
  Synthetic
WEDKRWSRMDQLAKEL

SEQ ID NO: 18 - protein, 15 aa, Artificial Sequence
  Synthetic
WEDKRWSRMDQLAKE

SEQ ID NO: 19 - protein, 14 aa, Artificial Sequence
  Synthetic
WEDKRWSRMDQLAK

SEQ ID NO: 20 - protein, 13 aa, Artificial Sequence
  Synthetic
WEDKRWSRMDQLA

SEQ ID NO: 21 - protein, 10 aa, Artificial Sequence
  Synthetic
SRLGLWVRME

SEQ ID NO: 22 - protein, 10 aa, Artificial Sequence
  Synthetic
SRLVLWVRME

SEQ ID NO: 23 - protein, 10 aa, Artificial Sequence
  Synthetic
SRLTLWVRME

SEQ ID NO: 24 - protein, 10 aa, Artificial Sequence
  Synthetic
SRLSLWVRME

SEQ ID NO: 25 - protein, 10 aa, Artificial Sequence
  Synthetic
SRLALWVRME

SEQ ID NO: 26 - protein, 10 aa, Artificial Sequence
  Synthetic
SRLPLWVRME

SEQ ID NO: 27 - protein, 10 aa, Artificial Sequence
  Synthetic
SRLCLWVRME

SEQ ID NO: 28 - protein, 10 aa, Artificial Sequence
  Synthetic
SRLYLWVRME

SEQ ID NO: 29 - protein, 10 aa, Artificial Sequence
  Synthetic
SRLRLWVRME

SEQ ID NO: 30 - protein, 10 aa, Artificial Sequence
  Synthetic
SRLMLWVRME

SEQ ID NO: 31 - protein, 10 aa, Artificial Sequence
  Synthetic
SRLHLWVRME

SEQ ID NO: 32 - protein, 10 aa, Artificial Sequence
  Synthetic
SRFGLWVRME

SEQ ID NO: 33 - protein, 9 aa, Artificial Sequence
  Synthetic
HRPIWARMD

SEQ ID NO: 34 - protein, 9 aa, Artificial Sequence
  Synthetic
HLAIWAKMD

SEQ ID NO: 35 - protein, 9 aa, Artificial Sequence
  Synthetic
HLAIWARMD

SEQ ID NO: 36 - protein, 9 aa, Artificial Sequence
  Synthetic
HIPIWARMD

SEQ ID NO: 37 - protein, 9 aa, Artificial Sequence
  Synthetic
RLGLWVRME

SEQ ID NO: 38 - protein, 9 aa, Artificial Sequence
  Synthetic
RVGQWARME

SEQ ID NO: 39 - protein, 9 aa, Artificial Sequence
  Synthetic
RLGGWARMM

SEQ ID NO: 40 - protein, 9 aa, Artificial Sequence
  Synthetic
ELMEWWKMM

SEQ ID NO: 41 - protein, 9 aa, Artificial Sequence
  Synthetic
PRITWTRMG

SEQ ID NO: 42 - protein, 18 aa, Artificial Sequence
  Synthetic
AEDQELESLSAIEAELEK

SEQ ID NO: 43 - protein, 14 aa, Artificial Sequence
  Synthetic
SDFEEKKEEEGSAN

SEQ ID NO: 44 - protein, 10 aa, Artificial Sequence
  Synthetic
WEDKRWSRMD

SEQ ID NO: 45 - protein, 15 aa, Artificial Sequence
  Synthetic
SHLVEALYLVCGERG

SEQ ID NO: 46 - protein, 9 aa, Artificial Sequence
  Synthetic
EDKRWSRMD

SEQ ID NO: 47 - protein, 17 aa, Artificial Sequence
  Synthetic
RPSSREDSVEARSDFEE

SEQ ID NO: 48 - protein, 7 aa, Artificial Sequence
  Synthetic
WXRKMDE

SEQ ID NO: 49 - protein, 14 aa, Homo sapiens
WSRMDQLAKELTEA

SEQ ID NO: 50 - protein, 7 aa, Artificial Sequence
  Synthetic
WXRKMED

SEQ ID NO: 51 - protein, 5 aa, Artificial Sequence
  Synthetic
WEDKR

SEQ ID NO: 52 - protein, 5 aa, Artificial Sequence
  Synthetic
WSRMD

SEQ ID NO: 53 - protein, 4 aa, Artificial Sequence
  Synthetic
EDKR

SEQ ID NO: 54 - protein, 15 aa, Homo sapiens
WSRKMDQLAKELTAE

SEQ ID NO: 55 - protein, 13 aa, Artificial Sequence
  Synthetic
QWEDKRWSRMDQA

SEQ ID NO: 56 - protein, 17 aa, Artificial Sequence
  Synthetic
GGGMKRHGLDNYRGYSL

SEQ ID NO: 57 - protein, 12 aa, Artificial Sequence
  Synthetic
WEDKRWSRMDQL

SEQ ID NO: 58 - protein, 13 aa, Artificial Sequence
  Synthetic
WSRMDQLAKELTA

SEQ ID NO: 59 - protein, 10 aa, Artificial Sequence
  Synthetic
XRXXLXLXXE

SEQ ID NO: 60 - protein, 10 aa, Artificial Sequence
  Synthetic
XIXXVXVXXG

SEQ ID NO: 61 - protein, 10 aa, Artificial Sequence
  Synthetic
AVRPLWVRME

SEQ ID NO: 62 - protein, 10 aa, Artificial Sequence
  Synthetic
QVAPVWVRME

SEQ ID NO: 63 - protein, 10 aa, Artificial Sequence
  Synthetic
KRHGLDNYRG

SEQ ID NO: 64 - protein, 24 aa, Artificial Sequence
  Synthetic
SREWEDKRWSRMDQLAKELTAEKR

SEQ ID NO: 65 - protein, 11 aa, Artificial Sequence
  Synthetic
WSRMDQLAKEL

SEQ ID NO: 66 - protein, 8 aa, Artificial Sequence
  Synthetic
WSRMDQLA

SEQ ID NO: 67 - protein, 16 aa, Artificial Sequence
  Synthetic
ANERADLIAYLKQATK

SEQ ID NO: 68 - protein, 463 aa, Artificial Sequence
  Synthetic
MRSTAVLALLLCAGQVFALPVNSPMTKGDTKVMKCVLEVISDSLSKPSPMPVSPECLETLQGDERILSILRHQNLLKELQDLALQGAKERAQQPLKQQQPPKQQQQQQQQQQQEQQHSSFEDELSEVFENQSPDAKHRDAAAEVPSRDTMEKRKDSDKGQQDGFEATTEGPRPQAFPEPNQESPMMGDSESPGEDTATNTQSPTSLPSQEHVDPQATGDSERGLSAQQQARKAKQEEKEEEEEEEAVAREKAGPEEVPTAASSSHFHAGYKAIQKDDGQSDSQAVDGDGKTEASEALPSEGKGELEHSQQEEDGEEAMVGTPQGLFPQGGKGRELEHKQEEEEEEEERLSREWEDKRWSRMDQLAKELTAEKRLEGEDDPDRSMKLSFRTRAYGFRDPGPQLRRGWRPSSREDSVEARSDFEEKKEEEGSANRRAEDQELESLSAIEAELEKVAMQLQALRRG